(19) United States
(12) Patent Application Publication
(10) Pub. No.: US 2024/0101975 A1
(43) Pub. Date: Mar. 28, 2024

(54) Antibody Conjugates and Methods of Making and Using the Same

(71) Applicant: R.P. Scherer Technologies, LLC, Carson City, NV (US)

(72) Inventors: Yun Cheol Kim, Walnut Creek, CA (US); Chao Bai Huang, San Leandro, CA (US); David Rabuka, Kensington, CA (US)

(21) Appl. No.: 18/243,482

(22) Filed: Sep. 7, 2023

Related U.S. Application Data
(62) Division of application No. 17/532,797, filed on Nov. 22, 2021, now Pat. No. 11,788,066.
Application No. 17/532,797 is a division of application No. 15/495,431, filed on Apr. 24, 2017, now Pat. No. 11,208,632.
(60) Provisional application No. 62/327,906, filed on Apr. 26, 2016.

(51) Int. Cl. C12N 9/02; A61K 47/60; A61K 47/68; C07K 16/00; C07K 16/30

(52) U.S. Cl. CPC C12N 9/0051; A61K 47/60; A61K 47/6851; C07K 16/00; C07K 16/30; C12Y 108/99; C07K 2317/14; C07K 2317/515; C07K 2317/52; C07K 2317/56; C07K 2317/94; C07K 2319/40

(57) ABSTRACT

Antibodies that include a sulfatase motif-containing tag in a constant region of an immunoglobulin (Ig) light chain polypeptide are disclosed. The sulfatase motif can be converted by a formylglycine-generating enzyme (FGE) to produce a formylglycine (fGly)-modified Ig light chain polypeptide. An fGly-modified Ig light chain polypeptide of the antibody can be covalently and site-specifically bound to a moiety of interest to provide an antibody conjugate. The disclosure also encompasses methods of production of such tagged Ig light chain polypeptides, fGly-modified Ig light chain polypeptides, and antibody conjugates, as well as methods of use of same.

### CROSS-REFERENCE TO RELATED APPLICATION

This application is a divisional of U.S. application Ser. No. 17/532,797, filed Nov. 22, 2021, now U.S. Pat. No. 11,788,066, which is a divisional of U.S. application Ser. No. 15/495,431, filed Apr. 24, 2017, now U.S. Pat. No. 11,208,632, which claims priority benefit of U.S. provisional application Ser. No. 62/327,906, filed Apr. 26, 2016, which applications are incorporated herein by reference in their entirety.

### INCORPORATION-BY-REFERENCE OF MATERIAL ELECTRONICALLY SUBMITTED

A Sequence Listing is provided herewith as a Sequence Listing XML, “RDWD-015DIV2_SEQ_LIST_10-16-23” created on Oct. 16, 2023, and having a size of 659,968 bytes. The contents of the Sequence Listing XML are incorporated by reference herein in their entirety.

### INTRODUCTION

Antibodies find use in various diagnostic and therapeutic applications. Antibodies can also be used to deliver drugs. However, conjugation of a drug to an antibody can be difficult to control, resulting in a heterogeneous mixture of conjugates that differ in the number of drug molecules attached. This can make controlling the amount administered to a patient difficult.

### SUMMARY

Antibodies that include a sulfatase motif-containing tag in a constant region of an immunoglobulin (Ig) light chain polypeptide are disclosed. The sulfatase motif of the tag can be converted by a formylglycine-generating enzyme (FGE) to produce a formylglycine (fGly)-modified Ig light chain polypeptide. An fGly-modified Ig light chain polypeptide of the antibody can be covalently and site-specifically bound to a moiety of interest (i.e., a payload, e.g., drug) to provide an antibody conjugate. The disclosure also encompasses methods of production of such tagged Ig light chain polypeptides, fGly-modified Ig light chain polypeptides, and antibody conjugates, as well as methods of use of same.

Provided herein is an antibody that includes an immunoglobulin (Ig) light chain polypeptide containing in a constant region an amino acid sequence of the formula: X¹Z¹X²Z²X³Z³, wherein Z¹ is cysteine, serine, 2-formylglycine (fGly), or fGly′, wherein fGly′ is an fGly residue covalently bound to a payload; Z² is proline or alanine; Z³ is an aliphatic amino acid or a basic amino acid; X¹ is present or absent, and when present, can be any amino acid; and X² and X³ are each independently any amino acid, wherein the amino acid sequence is positioned in the Ig light chain polypeptide such that when Z¹ is fGly, conjugation of the fGly (e.g., in a composition containing the present antibody) with the payload provides an average molar ratio of payload to antibody of at least 0.5. In some embodiments, the constant region of the Ig light chain polypeptide includes the amino acid sequence:

(SEQ ID NO: 46)
TX¹Z¹X²Z²X³Z³VA;
(SEQ ID NO: 47)
TVX¹Z¹X²Z²X³Z³AA;
(SEQ ID NO: 48)
VAX¹Z¹X²Z²X³Z³AP;
(SEQ ID NO: 49)
AAX¹Z¹X²Z²X³Z³PSV;
(SEQ ID NO: 50)
APX¹Z¹X²Z²X³Z³SVF;
(SEQ ID NO: 51)
PSX¹Z¹X²Z²X³Z³VF;
(SEQ ID NO: 52)
KSX¹Z¹X²Z²X³Z³GT;
(SEQ ID NO: 53)
KSGX¹Z¹X²Z²X³Z³TA;
(SEQ ID NO: 54)
GTX¹Z¹X²Z²X³Z³AS;
(SEQ ID NO: 55)
TAX¹Z¹X²Z²X³Z³SVV;
(SEQ ID NO: 56)
LNX¹Z¹X²Z²X³Z³NF;
(SEQ ID NO: 57)
NNX¹Z¹X²Z²X³Z³FY;
(SEQ ID NO: 58)
NFX¹Z¹X²Z²X³Z³YP;
(SEQ ID NO: 59)
FYX¹Z¹X²Z²X³Z³PR;
(SEQ ID NO: 60)
WKVX¹Z¹X²Z²X³Z³DN;
(SEQ ID NO: 61)
VDX¹Z¹X²Z²X³Z³N[A/V];
(SEQ ID NO: 62)
N[A/V]X¹Z¹X²Z²X³Z³LQ;
(SEQ ID NO: 63)
[A/V]LX¹Z¹X²Z²X³Z³QS;
(SEQ ID NO: 64)
LQX¹Z¹X²Z²X³Z³SGN;
(SEQ ID NO: 65)
QSX¹Z¹X²Z²X³Z³GN;
(SEQ ID NO: 66)
QSGX¹Z¹X²Z²X³Z³NS;
(SEQ ID NO: 67)
GNX¹Z¹X²Z²X³Z³SQ;
(SEQ ID NO: 68)
NSX¹Z¹X²Z²X³Z³QE;
(SEQ ID NO: 69)
SQX¹Z¹X²Z²X³Z³ES;
(SEQ ID NO: 70)
QDSX¹Z¹X²Z²X³Z³KD;
(SEQ ID NO: 71)
KDX¹Z¹X²Z²X³Z³STY;
(SEQ ID NO: 72)
KDSX¹Z¹X²Z²X³Z³TY;
(SEQ ID NO: 73)
DSTX¹Z¹X²Z²X³Z³YS;
(SEQ ID NO: 74)
TYX¹Z¹X²Z²X³Z³SL;
(SEQ ID NO: 75)
EVTX¹Z¹X²Z²X³Z³HQ;
(SEQ ID NO: 76)
THX¹Z¹X²Z²X³Z³QG;
(SEQ ID NO: 77)
HQX¹Z¹X²Z²X³Z³GL;
(SEQ ID NO: 78)
QGX¹Z¹X²Z²X³Z³LSSP;
(SEQ ID NO: 79)
GLX¹Z¹X²Z²X³Z³SS;
(SEQ ID NO: 80)
GLSX¹Z¹X²Z²X³Z³SP;
(SEQ ID NO: 81)
GLSSX¹Z¹X²Z²X³Z³PV;
or
(SEQ ID NO: 82)
SPX¹Z¹X²Z²X³Z³VT.

([*/*] denotes alternative amino acids (or amino acid sequences) chosen from the amino acid residues (or sequences) separated by “/”.) In some embodiments, Z³ is arginine. In some embodiments, X¹ is present. In some embodiments, X¹ is glycine, leucine, isoleucine, methionine, histidine, tyrosine, valine, serine, cysteine or threonine. In some embodiments, X² and X³ are each independently serine, threonine, alanine, valine, glycine or cysteine.

In any embodiment, the Ig light chain polypeptide constant region may contain two or more of SEQ ID NOs:46-82.

In any embodiment, the antibody may specifically bind a tumor antigen on a cancer cell.

In some embodiments, X¹Z¹X²Z²X³Z³ is LCTPSR (SEQ ID NO:158) or LSTPSR (SEQ ID NO:159). In some embodiments, X¹Z¹X²Z²X³Z³ is selected from MCTPSR (SEQ ID NO:160), VCTPSR (SEQ ID NO:161), LCSPSR (SEQ ID NO:162), LCAPSR (SEQ ID NO:163), LCVPSR (SEQ ID NO:164), LCGPSR (SEQ ID NO:165), ICTPAR (SEQ ID NO:166), LCTPSK (SEQ ID NO:167), MCTPSK (SEQ ID NO:168), VCTPSK (SEQ ID NO:169), LCSPSK (SEQ ID NO:170), LCAPSK (SEQ ID NO:171), LCVPSK (SEQ ID NO:172), LCGPSK (SEQ ID NO:173), LCTPSA (SEQ ID NO:174), ICTPAA (SEQ ID NO:175), MCTPSA (SEQ ID NO:176), VCTPSA (SEQ ID NO:177), LCSPSA (SEQ ID NO:178), LCAPSA (SEQ ID NO:179), LCVPSA (SEQ ID NO:180), LCGPSA (SEQ ID NO:181), MSTPSR (SEQ ID NO:182), VSTPSR (SEQ ID NO:183), LSSPSR (SEQ ID NO:184), LSAPSR (SEQ ID NO:185), LSVPSR (SEQ ID NO:186), LSGPSR (SEQ ID NO:187), ISTPAR (SEQ ID NO:188), LSTPSK (SEQ ID NO:189), MSTPSK (SEQ ID NO:190), VSTPSK (SEQ ID NO:191), LSSPSK (SEQ ID NO:192), LSAPSK (SEQ ID NO:193), LSVPSK (SEQ ID NO:194), LSGPSK (SEQ ID NO:195), LSTPSA (SEQ ID NO:196), ISTPAA (SEQ ID NO:197), MSTPSA (SEQ ID NO:198), VSTPSA (SEQ ID NO:199), LSSPSA (SEQ ID NO:200), LSAPSA (SEQ ID NO:201), LSVPSA (SEQ ID NO:202), and LSGPSA (SEQ ID NO:203).

In some embodiments, the composition includes an fGly-modified antibody, wherein Z¹ of the antibody is fGly. In some embodiments, X¹Z¹X²Z²X³Z³ is L(fGly)TPSR (SEQ ID NO:157). In some embodiments, X¹Z¹X²Z²X³Z³ is selected from M(fGly)TPSR (SEQ ID NO:204), V(fGly)TPSR (SEQ ID NO:205), L(fGly)SPSR (SEQ ID NO:206), L(fGly)APSR (SEQ ID NO:207), L(fGly)VPSR (SEQ ID NO:208), L(fGly)GPSR (SEQ ID NO:209), I(fGly)TPAR (SEQ ID NO:210), L(fGly)TPSK (SEQ ID NO:211), M(fGly)TPSK (SEQ ID NO:212), V(fGly)TPSK (SEQ ID NO:213), L(fGly)SPSK (SEQ ID NO:214), L(fGly)APSK (SEQ ID NO:215), L(fGly)VPSK (SEQ ID NO:216), L(fGly)GPSK (SEQ ID NO:217), L(fGly)TPSA (SEQ ID NO:218), I(fGly)TPAA (SEQ ID NO:219), M(fGly)TPSA (SEQ ID NO:220), V(fGly)TPSA (SEQ ID NO:221), L(fGly)SPSA (SEQ ID NO:222), L(fGly)APSA (SEQ ID NO:223), L(fGly)VPSA (SEQ ID NO:224), and L(fGly)GPSA (SEQ ID NO:225).

In some embodiments, the composition includes an antibody conjugate including the antibody covalently bound to the payload, wherein Z¹ is fGly′. In some embodiments, X¹(fGly′)X²Z²X³Z³ is L(fGly′)TPSR (SEQ ID NO:226). In some embodiments, X¹(fGly′)X²Z²X³Z³ is selected from M(fGly′)TPSR (SEQ ID NO:227), V(fGly′)TPSR (SEQ ID NO:228), L(fGly′)SPSR (SEQ ID NO:229), L(fGly′)APSR (SEQ ID NO:230), L(fGly′)VPSR (SEQ ID NO:231), L(fGly′)GPSR (SEQ ID NO:232), I(fGly′)TPAR (SEQ ID NO:233), L(fGly′)TPSK (SEQ ID NO:234), M(fGly′)TPSK (SEQ ID NO:235), V(fGly′)TPSK (SEQ ID NO:236), L(fGly′)SPSK (SEQ ID NO:237), L(fGly′)APSK (SEQ ID NO:238), L(fGly′)VPSK (SEQ ID NO:239), L(fGly′)GPSK (SEQ ID NO:240), L(fGly′)TPSA (SEQ ID NO:241), I(fGly′)TPAA (SEQ ID NO:242), M(fGly′)TPSA (SEQ ID NO:243), V(fGly′)TPSA (SEQ ID NO:244), L(fGly′)SPSA (SEQ ID NO:245), L(fGly′)APSA (SEQ ID NO:246), L(fGly′)VPSA (SEQ ID NO:247), and L(fGly′)GPSA (SEQ ID NO:248).

In any embodiment, the antibody may be covalently bound to the payload via a hydrazone, oxime, semicarbazone, alkyl, alkenyl, acyloxy, hydrazinyl-indolyl, hydrazinyl-imidazoyl, hydrazinyl-pyrrolyl, hydrazinyl-furanyl or a pyrazolinone linkage.

In any embodiment the antibody may be covalently bound to the payload via a linking group. In some embodiments, the linking group comprises a 4-aminopiperidine derivative (4AP).

In any embodiment the payload may be selected from a drug, a detectable label, a water-soluble polymer, and a synthetic peptide. In some embodiments, the payload is a small molecule drug. In some embodiments, the small molecule drug is a cancer chemotherapeutic agent. In some embodiments, the cancer chemotherapeutic agent is an alkylating agent, a nitrosourea, an antimetabolite, an antitumor antibiotic, a vinca alkaloid, or a steroid hormone. In some embodiments, the water-soluble polymer is poly(ethylene glycol). In some embodiments, the detectable label is an imaging agent. In some embodiments, the payload is a viral fusion inhibitor.

In any embodiment, the constant region of the Ig light chain polypeptide may include the amino acid sequence:

(SEQ ID NO: 120)
TX¹(fGly′)X²Z²X³Z³VA;
(SEQ ID NO: 121)
TVX¹(fGly′)X²Z²X³Z³AA;
(SEQ ID NO: 122)
VAX¹(fGly′)X²Z²X³Z³AP;
(SEQ ID NO: 123)
AAX¹(fGly′)X²Z²X³Z³PSV;
(SEQ ID NO: 124)
APX¹(fGly′)X²Z²X³Z³SVF;
(SEQ ID NO: 125)
PSX¹(fGly′)X²Z²X³Z³VF;
(SEQ ID NO: 126)
KSX¹(fGly′)X²Z²X³Z³GT;
(SEQ ID NO: 127)
KSGX¹(fGly′)X²Z²X³Z³TA;
(SEQ ID NO: 128)
GTX¹(fGly′)X²Z²X³Z³AS;
(SEQ ID NO: 129)
TAX¹(fGly′)X²Z²X³Z³SVV;
(SEQ ID NO: 130)
LNX¹(fGly′)X²Z²X³Z³NF;
(SEQ ID NO: 131)
NNX¹(fGly′)X²Z²X³Z³FY;
(SEQ ID NO: 132)
NFX¹(fGly′)X²Z²X³Z³YP;
(SEQ ID NO: 133)
FYX¹(fGly′)X²Z²X³Z³PR;
(SEQ ID NO: 134)
WKVX¹(fGly′)X²Z²X³Z³DN;
(SEQ ID NO: 135)
VDX¹(fGly′)X²Z²X³Z³N[A/V];
(SEQ ID NO: 136)
N[A/V]X¹(fGly′)X²Z²X³Z³LQ;
(SEQ ID NO: 137)
[A/V]LX¹(fGly′)X²Z²X³Z³QS;
(SEQ ID NO: 138)
LQX¹(fGly′)X²Z²X³Z³SGN;
(SEQ ID NO: 139)
QSX¹(fGly′)X²Z²X³Z³GN;
(SEQ ID NO: 140)
QSGX¹(fGly′)X²Z²X³Z³NS;
(SEQ ID NO: 141)
GNX¹(fGly′)X²Z²X³Z³SQ;
(SEQ ID NO: 142)
NSX¹(fGly′)X²Z²X³Z³QE;
(SEQ ID NO: 143)
SQX¹(fGly′)X²Z²X³Z³ES;
(SEQ ID NO: 144)
QDSX¹(fGly′)X²Z²X³Z³KD;
(SEQ ID NO: 145)
KDX¹(fGly′)X²Z²X³Z³STY;
(SEQ ID NO: 146)
KDSX¹(fGly′)X²Z²X³Z³TY;
(SEQ ID NO: 147)
DSTX¹(fGly′)X²Z²X³Z³YS;
(SEQ ID NO: 148)
TYX¹(fGly′)X²Z²X³Z³SL;
(SEQ ID NO: 149)
EVTX¹(fGly′)X²Z²X³Z³HQ;
(SEQ ID NO: 150)
THX¹(fGly′)X²Z²X³Z³QG;
(SEQ ID NO: 151)
HQX¹(fGly′)X²Z²X³Z³GL;
(SEQ ID NO: 152)
QGX¹(fGly′)X²Z²X³Z³LSSP;
(SEQ ID NO: 153)
GLX¹(fGly′)X²Z²X³Z³SS;
(SEQ ID NO: 154)
GLSX¹(fGly′)X²Z²X³Z³SP;
(SEQ ID NO: 155)
GLSSX¹(fGly′)X²Z²X³Z³PV;
(SEQ ID NO: 156)
SPX¹(fGly′)X²Z²X³Z³VT.

Also provided herein is an antibody conjugate containing an antibody covalently bound to a payload, the antibody containing an immunoglobulin (Ig) light chain polypeptide including, in a constant region, an amino acid sequence of the formula: X¹(fGly′)X²Z²X³Z³, wherein fGly′ is an fGly residue covalently bound to the payload; Z² is proline or alanine; Z³ is an aliphatic amino acid or a basic amino acid; X¹ is present or absent, and when present, can be any amino acid; and X² and X³ are each independently any amino acid, and wherein the constant region of the Ig light chain polypeptide includes the amino acid sequence:

(SEQ ID NO: 120)
TX¹(fGly′)X²Z²X³Z³VA;
(SEQ ID NO: 121)
TVX¹(fGly′)X²Z²X³Z³AA;
(SEQ ID NO: 122)
VAX¹(fGly′)X²Z²X³Z³AP;
(SEQ ID NO: 123)
AAX¹(fGly′)X²Z²X³Z³PSV;
(SEQ ID NO: 124)
APX¹(fGly′)X²Z²X³Z³SVF;
(SEQ ID NO: 125)
PSX¹(fGly′)X²Z²X³Z³vF;
(SEQ ID NO: 126)
KSX¹(fGly′)X²Z²X³Z³GT;
(SEQ ID NO: 127)
KSGX¹(fGly′)X²Z²X³Z³TA;
(SEQ ID NO: 128)
GTX¹(fGly′)X²Z²X³Z³AS;
(SEQ ID NO: 129)
TAX¹(fGly′)X²Z²X³Z³SVV;
(SEQ ID NO: 130)
LNX¹(fGly′)X²Z²X³Z³NF;
(SEQ ID NO: 131)
NNX¹(fGly′)X²Z²X³Z³FY;
(SEQ ID NO: 132)
NFX¹(fGly′)X²Z²X³Z³YP;
(SEQ ID NO: 133)
FYX¹(fGly′)X²Z²X³Z³PR;
(SEQ ID NO: 134)
WKVX¹(fGly′)X²Z²X³Z³DN;
(SEQ ID NO: 135)
VDX¹(fGly′)X²Z²X³Z³N[A/V];
(SEQ ID NO: 136)
N[A/V]X¹(fGly′)X²Z²X³Z³LQ;
(SEQ ID NO: 137)
[A/V]LX¹(fGly′)X²Z²X³Z³QS;
(SEQ ID NO: 138)
LQX¹(fGly′)X²Z²X³Z³SGN;
(SEQ ID NO: 139)
QSX¹(fGly′)X²Z²X³Z³GN;
(SEQ ID NO: 140)
QSGX¹(fGly′)X²Z²X³Z³NS;
(SEQ ID NO: 141)
GNX¹(fGly′)X²Z²X³Z³SQ;
(SEQ ID NO: 142)
NSX¹(fGly′)X²Z²X³Z³QE;
(SEQ ID NO: 143)
SQX¹(fGly′)X²Z²X³Z³ES;
(SEQ ID NO: 144)
QDSX¹(fGly′)X²Z²X³Z³KD;
(SEQ ID NO: 145)
KDX¹(fGly′)X²Z²X³Z³STY;
(SEQ ID NO: 146)
KDSX¹(fGly′)X²Z²X³Z³TY;
(SEQ ID NO: 147)
DSTX¹(fGly′)X²Z²X³Z³YS;
(SEQ ID NO: 148)
TYX¹(fGly′)X²Z²X³Z³SL;
(SEQ ID NO: 149)
EVTX¹(fGly′)X²Z²X³Z³HQ;
(SEQ ID NO: 150)
THX¹(fGly′)X²Z²X³Z³QG;
(SEQ ID NO: 151)
HQX¹(fGly′)X²Z²X³Z³GL;
(SEQ ID NO: 152)
QGX¹(fGly′)X²Z²X³Z³LSSP;
(SEQ ID NO: 153)
GLX¹(fGly′)X²Z²X³Z³SS;
(SEQ ID NO: 154)
GLSX¹(fGly′)X²Z²X³Z³SP;
(SEQ ID NO: 155)
GLSSX¹(fGly′)X²Z²X³Z³PV;
or
(SEQ ID NO: 156)
SPX¹(fGly′)X²Z²X³Z³VT.

Also provided herein is a recombinant nucleic acid that includes a nucleotide sequence encoding the light chain constant region of the antibody of the present disclosure, wherein Z¹ is cysteine or serine. Also provided herein is a recombinant expression vector containing the nucleic acid encoding the light chain constant region of the antibody of the present disclosure, wherein the light chain constant region-encoding nucleotide sequence is operably linked to a promoter. In some embodiments, the recombinant expression vector further includes a nucleotide sequence encoding an Ig heavy chain polypeptide.

Also provided herein is a host cell genetically modified to express the antibody of the present disclosure, wherein Z¹ is cysteine, serine or fGly. In some embodiments, the host cell is genetically modified to express a formylglycine generating enzyme (FGE), in a manner sufficient to convert an Ig light chain polypeptide of the antibody into an fGly-modified Ig light chain polypeptide. In some embodiments, the host cell is a mammalian cell.

Also provided herein is a method of producing an antibody conjugate, including: combining, in a reaction mixture: the composition of any of the embodiments above containing an fGly-modified antibody; and a reactive partner containing a payload and an aldehyde-reactive group, under conditions sufficient for the aldehyde-reactive group to react with an aldehyde group of the fGly residue of the fGly-modified antibody, thereby conjugating the payload to the fGly residue via a covalent linkage to generate an antibody conjugate; and isolating the antibody conjugate from the reaction mixture. In some embodiments, the aldehyde-reactive group is selected from the group consisting of: a hydrazine, hydrazide, aminooxy, semicarbazide, hydrazinyl-indole, hydrazinyl-imidazole, hydrazinyl-pyrrole, hydrazinyl-furan and a pyrazolinone group. In some embodiments reactive partner includes a linking group covalently linking the aldehyde-reactive group with the payload. In some embodiments the linking group includes a 4-aminopiperidine derivative (4AP).

Also provided herein is a formulation that includes: the composition of any of the embodiments above containing an antibody conjugate, or the antibody conjugate of the present disclosure; and a pharmaceutically acceptable excipient.

Also provided is a method of treating an individual for cancer, by administering to an individual a therapeutically effective amount of the composition of any of the embodiments above containing an antibody conjugate or the antibody conjugate of the present disclosure.

### BRIEF DESCRIPTION OF THE DRAWINGS

FIG. 1 shows a schematic illustrating scanning insertion of a sulfatase motif (LCTPSR) in a human immunoglobulin (Ig) kappa light chain constant region amino acid sequence (SEQ ID NOS:1-14), according to embodiments of the present disclosure.

FIGS. 2A and 2B is a graph showing the expression titer (y-axis) of an antigen-specific antibody modified with a sulfatase motif insertion adjacent and C-terminal to the indicated position, as defined relative to SEQ ID NO:1, in the constant region of its Ig light chain amino acid sequence, according to embodiments of the present disclosure. The sulfatase motif is inserted immediately after the amino acid position indicated, as shown schematically in FIG. 1.

FIG. 3 is a table listing percentage of aggregation, measured by size exclusion chromatography (SEC), of an antigen-specific antibody modified with a sulfatase motif insertion adjacent and C-terminal to the indicated position, as defined relative to SEQ ID NO:1, in the constant region of its Ig light chain amino acid sequence, according to embodiments of the present disclosure.

FIG. 4 is a graph showing the drug-to-antibody ratio (DAR) for an antigen-specific antibody conjugated to a hydrophobic payload, where the antibody was modified by having a sulfatase motif inserted adjacent and C-terminal to the indicated position, as defined relative to SEQ ID NO:1, in the constant region of its Ig light chain amino acid sequence, according to embodiments of the present disclosure.

FIG. 5 is a graph showing DAR of for an antigen-specific antibody conjugated to a hydrophobic payload, where the antibody was modified by having a sulfatase motif inserted adjacent and C-terminal to the indicated position, as defined relative to SEQ ID NO:1, in the constant region of its Ig light chain amino acid sequence, according to embodiments of the present disclosure.

FIGS. 6A-6E are a collection of graphs showing antigen binding activity of an antigen-specific antibody conjugated to a cytotoxic drug, where the antibody was modified by having a sulfatase motif inserted adjacent and C-terminal to the indicated position, as defined relative to SEQ ID NO:1, in the constant region of its Ig light chain amino acid sequence, according to embodiments of the present disclosure.

FIGS. 7A-7B are a collection of graphs showing efficacy of a cytotoxic drug conjugated to an antigen-specific antibody having a sulfatase motif inserted adjacent and C-terminal to the indicated position, as defined relative to SEQ ID NO:1, in the constant region of its Ig light chain amino acid sequence, according to embodiments of the present disclosure.

FIGS. 8A-8C is a table comparing solvent accessible loop regions in relation to the DAR observed upon conjugation of an antigen-specific antibody to a cytotoxic drug (hydrophobic payload), where the antibody was modified by having a sulfatase motif inserted adjacent and C-terminal to the indicated position, as defined relative to SEQ ID NO:1, in the constant region of its Ig light chain amino acid sequence, according to embodiments of the present disclosure.

FIG. 9 shows an amino acid sequence of constant regions of human immunoglobulin kappa light chain polypeptide (SEQ ID NO:1), and location of amino acid variations present in different allotypes (“Km*”).

FIGS. 10A-10D show amino acid sequences of human Ig kappa light chain constant region amino acid sequence, with or without an inserted sulfatase motif (underlined), according to embodiments of the present disclosure.

FIG. 11 shows a vector map of an expression vector encoding a light chain polypeptide of an antigen-specific antibody, according to embodiments of the present disclosure.

FIG. 12 shows a vector map for an expression vector encoding a heavy chain polypeptide of an antigen-specific antibody.

FIGS. 13A and 13B are a collection of schematic diagrams showing the PCR primers and assembly strategy of DNA fragments, according to embodiments of the present disclosure.

FIGS. 14A-14E shows Table 3, listing the primer sets used to amplify clones, according to embodiments of the present disclosure.

FIG. 15 shows Table 4, listing the conjugation results for light chain tagged antibodies, according to embodiments of the present disclosure.

### DEFINITIONS

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are typical of measurements characterizing the disclosed compositions or appropriate to perform the disclosed methods.

The terms “polypeptide,” “peptide,” and “protein” are used interchangeably herein to refer to a polymeric form of amino acids of any length. Unless specifically indicated otherwise, “polypeptide,” “peptide,” and “protein” can include genetically coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, proteins which contain at least one N-terminal methionine residue (e.g., to facilitate production in a recombinant bacterial host cell); immunologically tagged proteins; and the like.

“Native amino acid sequence” or “parent amino acid sequence” are used interchangeably herein in the context of an immunoglobulin to refer to the amino acid sequence of the immunoglobulin prior to modification to include a heterologous aldehyde tag.

The term “antibody” is used in the broadest sense and includes monoclonal antibodies, and multispecific antibodies (e.g., bispecific antibodies), humanized antibodies, chimeric antibodies, and antigen-binding antibody fragments (e.g., Fab fragments). A target antigen can have one or more binding sites, also called epitopes, recognized by complementarity determining regions (CDRs) formed by one or more variable regions of an antibody.

“Immunoglobulin polypeptide” as used herein refers to a polypeptide comprising at least a constant region of a light chain polypeptide or at least a constant region of a heavy chain polypeptide.

An immunoglobulin light or heavy chain polypeptide variable region is composed of a framework region (FR) interrupted by three hypervariable regions, also called “complementarity determining regions” or “CDRs”. The extent of the framework region and CDRs have been defined (see, “Sequences of Proteins of Immunological Interest,” E. Kabat et al., U.S. Department of Health and Human Services, 1991). The framework region of an antibody, that is the combined framework regions of the constituent light and heavy chains, serves to position and align the CDRs. The CDRs are primarily responsible for binding to an epitope of an antigen. An immunoglobuline light chain may have a structure schematically represented, from N- to C-termini, as: FR1-CDR1-FR2-CDR2-FR3-CDR3-FR4-C_{L}, where CDR1, CDR2 and CDR3 are hypervariable regions that interrupt the framework region into four (FR1, FR2, FR3 and FR4) and C_{L} is the constant region. An immunoglobuline heavy chain may have a structure schematically represented, from N- to C-termini, as: FR1-CDR1-FR2-CDR2-FR3-CDR3-FR4-C_{H}1-H-C_{H}2-CH3, where CDR1, CDR2 and CDR3 are hypervariable regions that interrupt the framework region into four (FR1, FR2, FR3 and FR4), C_{H}1, C_{H}2 and C_{H}3 are constant regions and H is a hinge region.

The term “natural antibody” refers to an antibody in which the heavy and light chains of the antibody have been made and paired by the immune system of a multi-cellular organism. Spleen, lymph nodes, bone marrow and serum are examples of tissues that produce natural antibodies. For example, the antibodies produced by the antibody producing cells isolated from a first animal immunized with an antigen are natural antibodies.

A “parent Ig polypeptide” is a polypeptide comprising an amino acid sequence which lacks a tagged constant region as described herein. The parent polypeptide may comprise a native sequence constant region, or may comprise a constant region with pre-existing amino acid sequence modifications (such as additions, deletions and/or substitutions).

In the context of an Ig polypeptide, the term “constant region” is well understood in the art, and refers to a C-terminal region of an Ig heavy chain, or an Ig light chain. An Ig heavy chain constant region includes CH1, CH2, and CH3 domains (and CH4 domains, where the heavy chain is a or an F heavy chain). In a native Ig heavy chain, the CH1, CH2, CH3 (and, if present, CH4) domains begin immediately after (C-terminal to) the heavy chain variable (V_{H}) region, and are each from about 100 amino acids to about 130 amino acids in length. In a native Ig light chain, the constant region begins begin immediately after (C-terminal to) the light chain variable (V_{L}) region, and is about 100 amino acids to 120 amino acids in length.

In some embodiments, a “functional Fc region” possesses an “effector function” of a native sequence Fc region. Exemplary “effector functions” include C1q binding; complement dependent cytotoxicity; Fc receptor binding; antibody-dependent cell-mediated cytotoxicity (ADCC); phagocytosis; down-regulation of cell surface receptors (e.g. B cell receptor; BCR), etc. Such effector functions generally require the Fc region to be combined with a binding domain (e.g. an antibody variable domain) and can be assessed using various assays that are well known in the art.

Antibody-dependent cell-mediated cytotoxicity” and “ADCC” refer to a cell-mediated reaction in which nonspecific cytotoxic cells that express FcRs (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. The terms “Fc receptor” or “FcR” are used to describe a receptor that binds to the Fc region of an antibody.

The term “humanized antibody” or “humanized immunoglobulin” refers to a non-human (e.g., mouse or rabbit) antibody containing one or more amino acids (in a framework region, a constant region or a CDR, for example) that have been substituted with a correspondingly positioned amino acid from a human antibody. In general, humanized antibodies produce a reduced immune response in a human host, as compared to a non-humanized version of the same antibody. Antibodies can be humanized using a variety of techniques known in the art including, for example, CDR-grafting, veneering or resurfacing, and chain shuffling. In certain embodiments, framework substitutions are identified by modeling of the interactions of the CDR and framework residues to identify framework residues important for antigen binding and sequence comparison to identify unusual framework residues at particular positions.

The term “chimeric antibodies” refer to antibodies whose light and heavy chain genes have been constructed, typically by genetic engineering, from antibody variable and constant region genes belonging to different species. For example, the variable segments of the genes from a mouse monoclonal antibody may be joined to human constant segments, such as gamma 1 and gamma 3. An example of a therapeutic chimeric antibody is a hybrid protein composed of the variable or antigen-binding domain from a mouse antibody and the constant or effector domain from a human antibody, although domains from other mammalian species may be used.

By “genetically-encodable” as used in reference to an amino acid sequence of polypeptide, peptide or protein means that the amino acid sequence is composed of amino acid residues that are capable of production by transcription and translation of a nucleic acid encoding the amino acid sequence, where transcription and/or translation may occur in a cell or in a cell-free in vitro transcription/translation system.

The term “control sequences” refers to DNA sequences that facilitate expression of an operably linked coding sequence in a particular expression system, e.g. mammalian cell, bacterial cell, cell-free synthesis, etc. The control sequences that are suitable for prokaryote systems, for example, include a promoter, optionally an operator sequence, and a ribosome binding site. Eukaryotic cell systems may utilize promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate the initiation of translation. Generally, “operably linked” means that the DNA sequences being linked are contiguous, and, in the case of a secretory leader, contiguous and in reading frame. Linking is accomplished by ligation or through amplification reactions. Synthetic oligonucleotide adaptors or linkers may be used for linking sequences in accordance with conventional practice.

The term “expression cassette” as used herein refers to a segment of nucleic acid, usually DNA, that can be inserted into a nucleic acid (e.g., by use of restriction sites compatible with ligation into a construct of interest or by homologous recombination into a construct of interest or into a host cell genome). In general, the nucleic acid segment comprises a polynucleotide that encodes a polypeptide of interest (e.g., a tagged Ig protein), and the cassette and restriction sites are designed to facilitate insertion of the cassette in the proper reading frame for transcription and translation. Expression cassettes can also comprise elements that facilitate expression of a polynucleotide encoding a polypeptide of interest in a host cell. These elements may include, but are not limited to: a promoter, a minimal promoter, an enhancer, a response element, a terminator sequence, a polyadenylation sequence, and the like.

As used herein the term “isolated” is meant to describe a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “substantially purified” refers to a compound that is removed from its natural environment and is at least 60% free, at least 75% free, at least 80% free, at least 85% free, at least 90% free, at least 95% free, at least 98% free, or more than 98% free, from other components with which it is naturally associated.

The term “physiological conditions” is meant to encompass those conditions compatible with living cells, e.g., predominantly aqueous conditions of a temperature, pH, salinity, etc. that are compatible with living cells.

“N-terminus” refers to the terminal amino acid residue of a polypeptide having a free amine group, which amine group in non-N-terminus amino acid residues normally forms part of the covalent backbone of the polypeptide.

“C-terminus” refers to the terminal amino acid residue of a polypeptide having a free carboxyl group, which carboxyl group in non-C-terminus amino acid residues normally forms part of the covalent backbone of the polypeptide.

By “internal site” as used in referenced to a polypeptide or an amino acid sequence of a polypeptide means a region of the polypeptide that is not at the N-terminus or at the C-terminus.

As used herein, the terms “treat,” “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

A “therapeutically effective amount” refers to an amount effective, at dosages and for periods of time necessary, to achieve the desired therapeutic result. A therapeutically effective amount of a therapeutic agent may vary according to factors such as the disease state, age, sex, and weight of the individual, and the ability of the therapeutic agent to elicit a desired response in the individual. A therapeutically effective amount is also one in which any toxic or detrimental effects of the therapeutic agent are outweighed by the therapeutically beneficial effects.

By “tag” is meant an amino acid sequence that contains an amino acid sequence motif found in sulfatases (hereinafter “sulfatase motif”), which amino acid sequence motif is capable of being converted, by action of a formylglycine generating enzyme (FGE), to contain a 2-formylglycine residue (referred to herein as “fGly”). The fGly residue generated by an FGE is often referred to in the literature as a “formylglycine”. Stated differently, the term “tag” is used herein to refer to an amino acid sequence comprising an “unconverted” sulfatase motif (i.e., a sulfatase motif in which the cysteine or serine residues has not been converted to fGly by an FGE, but is capable of being converted). The sulfatase motif may be exchangeable with “FGE substrate motif”. A “tagged” polypeptide contains an amino acid sequence motif, e.g., a sulfatase motif, that can be converted by an FGE to contain fGly.

By “conversion” as used in the context of action of a formylglycine generating enzyme (FGE) on a sulfatase motif refers to biochemical modification of a cysteine or serine residue in a sulfatase motif to a formylglycine (fGly) residue (e.g., Cys to fGly, or Ser to fGly).

“Aldehyde tag” or “ald-tag” as used herein, may refer to a tag that contains a sulfatase motif, which has been converted, by action of an FGE, to contain fGly. A converted tag refers to an amino acid sequence comprising a “converted” sulfatase motif (i.e., a sulfatase motif in which the cysteine or the serine residue has been converted to fGly by action of an FGE). An “aldehyde tagged” polypeptide contains an amino acid sequence motif, e.g., a sulfatase motif, that has been converted by an FGE to contain fGly.

By “reactive partner” is meant a molecule or molecular moiety that specifically reacts with another reactive partner to produce a reaction product. Exemplary reactive partners include a cysteine or serine of sulfatase motif and an FGE, which react to form a reaction product of a converted aldehyde tag containing an fGly in lieu of cysteine or serine in the motif. Other exemplary reactive partners include an aldehyde of a formylglycine (fGly) residue of a converted aldehyde tag and an “aldehyde-reactive reactive partner”, which comprises an aldehyde-reactive group and a moiety of interest (i.e., a payload, e.g., drug), and which reacts to form a reaction product of a modified aldehyde tagged polypeptide having the payload (e.g., drug) conjugated to the fGly-modified polypeptide via an fGly residue.

By “conjugate” is meant a first moiety that is stably associated with a second moiety. By “stably associated” is meant that a moiety is bound to another moiety or structure under standard conditions. In certain embodiments, the first and second moieties are bound to each other through one or more covalent bonds. The first or the second moiety of a conjugate may be referred to as a “payload”.

Before the present disclosure is further described, it is to be understood that the disclosed subject matter is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosed subject matter. The upper and lower limits of these smaller ranges may independently be included in the smaller ranges, and are also encompassed within the disclosed subject matter, subject to any specifically excluded limit in the stated range. Where the stated range includes one or both of the limits, ranges excluding either or both of those included limits are also included in the disclosed subject matter.

Unless defined otherwise, all technical and scientific terms used herein have the same meaning as commonly understood by one of ordinary skill in the art to which the disclosed subject matter belongs. Although any methods and materials similar or equivalent to those described herein can also be used in the practice or testing of the disclosed subject matter, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an antibody” includes a plurality of such antibodies and reference to “the antigen” includes reference to one or more antigens and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the disclosed subject matter, which are, for clarity, described in the context of separate embodiments, may also be provided in combination in a single embodiment. Conversely, various features of the disclosed subject matter, which are, for brevity, described in the context of a single embodiment, may also be provided separately or in any suitable sub-combination. All combinations of the embodiments pertaining to the disclosure are specifically embraced by the disclosed subject matter and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the disclosed subject matter is not entitled to antedate such publication by virtue of prior invention. Further, the dates of publication provided may be different from the actual publication dates which may need to be independently confirmed.

### DETAILED DESCRIPTION

As summarized above, an antibody that includes a tag, e.g., a tag containing a sulfatase motif, in an immunoglobulin (Ig) light chain polypeptide is disclosed. The tag includes a substrate motif for a formylglycine-generating enzyme (FGE), where FGE can convert (oxidize) a serine or cysteine residue in the substrate motif to a 2-formylglycine residue (fGly), thereby generating an fGly-modified antibody. An fGly-modified antibody can further react with an aldehyde-reactive partner to generate an antibody conjugate, where a moiety of interest (i.e., a payload, e.g., drug) is bound covalently and site-specifically to the light chain via the fGly.

The tagged antibodies, conjugates, compositions and methods of the present disclosure exploit a naturally-occurring, genetically-encodable sulfatase motif for use as a tag, referred to herein as a “tag”, to direct site-specific modification of an Ig polypeptide. The sulfatase motif of the tag, which motif is based on a motif found in active sites of sulfatases, contains a serine or cysteine residue that is capable of being converted (oxidized) to a 2-formylglycine (fGly) residue by action of a formylglycine generating enzyme (FGE) either in a cell-based system in an FGE-expressing host cell (e.g., at the time of translation of an ald tag-containing protein in a cell) or in a cell-free system (e.g., by contacting an ald tag-containing protein with an FGE in a cell-free system). The aldehyde moiety of the resulting fGly residue can be used as a “chemical handle” to facilitate site-specific chemical modification of the Ig polypeptide, and thus site-specific attachment of a payload (e.g., drug). For example, a peptide modified to contain an α-nucleophile-containing moiety (e.g., an aminooxy or hydrazide moiety) can be reacted with the fGly-containing Ig polypeptide to yield a conjugate in which the Ig polypeptide and the peptide are linked by a covalent bond, e.g., a hydrazone or oxime bond, or via alternative aldehyde-specific chemistries such as reductive amination, etc. The reactivity of the aldehyde thus allows for bioorthogonal and chemoselective modification of the Ig polypeptide, and thus provides a site-specific means for chemical modification that in turn can be exploited to provide for site-specific attachment of a payload in the final conjugate.

Tags may be positioned in an Ig light chain polypeptide in any suitable manner such that the tagged Ig light chain polypeptide, an antibody having the tagged light chain polypeptide, or both exhibit one or more desirable properties. The properties may be associated with, e.g., the tagged and/or the fGly-modified antibody produced in vitro (i.e., in a cellular expression system), and/or the antibody conjugate having a payload (e.g., drug) covalently bound to the antibody through fGly. The desirable properties may include, without limitation, higher titer of antibody production, higher conversion rate, higher conjugation yield (e.g., as measured by the average molar ratio of payload to antibody, e.g., drug to antibody), lower aggregation rate, lower immunogenicity, and/or higher stability in serum, relative to reference measures for the respective properties. A tagged, fGly-modified or conjugated antibody of the present disclosure may be characterized in satisfying one or more threshold criteria, e.g., two or more threshold criteria, such as expression titer and/or conjugation yield (e.g., payload-to-antibody ratio (PAR), e.g., the drug-to-antibody ratio, or DAR, where the payload is a drug) that are higher than a threshold titer and/or a threshold yield, respectively.

A tagged or fGly-modified antibody of the present disclosure may exhibit a desirable titer of expression. “Titer of expression”, “expression titer” and “titer” are used herein interchangeably, in reference to an antibody, to refer to the amount of antibody secreted in a cell culture supernatant by cultured cells that are genetically modified with suitable expression constructs encoding the antibody. The cells may be genetically modified to coexpress any convenient Ig heavy chain polypeptide with the tagged Ig light chain polypeptide. The cells may be further genetically modified with additional expression constructs encoding enzymes, or any other suitable polypeptide. In some cases, the cells may be genetically modified to express a formylglycine generating enzyme (FGE), as described herein. The threshold titer of expression may be, in some cases, about 5 mg/L or more, e.g., about 6 mg/L or more, about 8 mg/L or more, about 10 mg/L or more, about 15 mg/L or more, about 20 mg/L or more, about 30 mg/L or more, or about 40 mg/L or more. In some embodiments, the threshold titer of expression is in the range of about 5 to about 500 mg/L, e.g., about 6 to about 500 mg/L, about 8 to about 200 mg/L, about 10 to about 200 mg/L, about 20 to about 200 mg/L, or about 30 to about 200 mg/L. The antibody titer may be measured using, e.g., a biosensor chip system, such as a protein A-based biosensor assay run on the BLItz® system (Forte Bio, CA).

A tagged or fGly-modified antibody of the present disclosure may exhibit an acceptable level of aggregation in solution. Aggregation may refer to a non-covalent or covalent interaction between antibodies that causes two or more antibodies to physically associate with one another. Thus, the aggregation level of the tagged or fGly-modified antibody may be sufficiently low so as not to interfere with the antigen binding properties, the conjugation yield (as described below), the immunogenicity, etc., of the antibody. In some cases, the tagged or fGly-modified antibody exhibits an aggregation level of about 20% or less, e.g., about 18% or less, about 16% or less, about 14% or less, about 12% or less, about 10% or less, about 5% or less, about 3% or less, about 2% or less, including about 1% or less. In some cases, the tagged or fGly-modified antibody exhibits an aggregation level in the range of about 0% to about 20%, e.g., about 0% to about 16%, about 1% to about 12%, including about 1% to about 10%. Aggregation levels may be measured using, e.g., size exclusion chromatography.

An fGly-modified antibody that includes a converted tag present in each Ig light chain polypeptide constant region, as disclosed herein, may exhibit a desirable conjugation yield as expressed by the average molar ratio of payload to antibody (PAR) (e.g., drug-antibody ratio (DAR), where the payload is a drug), when the fGly-modified antibody is conjugated with a payload, such as a drug, through the fGly in a suitable reaction mixture. The payload prior to conjugation with the fGly-modified antibody may be covalently attached to a suitable reactive group, e.g., an aldehyde-reactive group, that reacts with the aldehyde of the fGly residue of the fGly-modified antibody in the reaction mixture under suitable conditions. In some embodiments, the conjugation yield is about 0.5 or more, e.g., about 0.75 or more, about 1.0 or more, about 1.1 or more, about 1.2 or more, about 1.3 or more, or about 1.6 or more, and up to 2.0. In some embodiments, the conjugation yield is in the range of about 0.5 to about 2.0, e.g., about 0.5 to about 1.9, about 0.75 to about 1.8, about 1.0 to about 1.8, or about 1.3 to about 1.8. The conjugation yield may be measured by performing, e.g., hydrophobic interaction chromatography (HIC), after a conjugation reaction.

An antibody conjugate of the present disclosure may bind an antigen with a suitable binding activity (e.g., specificity, binding affinity, etc.) compared to the parent antibody (i.e., the antibody without a payload conjugated thereto, or the antibody having an Ig light chain polypeptide without the tag sequence inserted in the constant region). In some cases, the antibody conjugate has binding activity toward an antigen that is substantially the same as the binding activity of a parent antibody that does not have a tag sequence inserted in the constant region of the Ig light chain polypeptide. The binding activity may be measured by, e.g., an enzyme-linked immunosorbent assay (ELISA).

The present antibody conjugates may find use in delivering a conjugated payload (e.g., drug) to a target site, where the antibody conjugate may bind specifically to an antigen specific for, or enriched at, the target site. For example, the antibody conjugate may specifically recognize a tumor antigen and enhance site-specific delivery of a chemotherapeutic drug conjugated to the antibody to the tumor.

Further aspects of the present disclosure are now described.

Tags Containing a Sulfatase Motif

An antibody of the present disclosure includes a tag, i.e., includes an amino acid sequence containing a sulfatase motif which is capable of being converted, by action of FGE, to provide a fGly in the sulfatase motif, in an Ig light chain polypeptide constant region. The tag may include a sulfatase motif having a length of 5 amino acid residues or more, e.g., 6 amino acid residues or more, 7 amino acid residues or more, 8 amino acid residues or more, including 10 amino acid residues or more, and in some cases may have a length of 15 amino acid residues or less, e.g., 12 amino acid residues or less, 11 amino acid residues or less, 10 amino acid residues or less, including 8 amino acid residues or less. In some embodiments, the tag includes a sulfatase motif having a length in the range of 5 to 15 amino acid residues, e.g., 5 to 12 amino acid residues, 5 to 10 amino acid residues, including 6 to 8 residues. In some embodiments, the sulfatase motif includes 5 or 6 amino acid residues.

In some embodiments, the tag includes at least a minimal sulfatase motif (also referred to a “consensus sulfatase motif”), having 5 or 6 amino acid residues, and additional sequence flanking the minimal sulfatase motif. The additional sequence may be N- and/or C-terminal to the minimal sulfatase motif.

In certain embodiments, the sulfatase motif may be described by the formula:

X¹Z¹X²Z²X³Z³ (I)

where Z¹ is cysteine or serine (which can also be represented by (C/S)); Z² is either a proline or alanine residue (which can also be represented by (P/A)); Z³ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), usually A, G, L, V, or I; X¹ is present or absent and, when present, can be any amino acid, though usually an aliphatic amino acid, a sulfur-containing amino acid, or a polar, uncharged amino acid, (i.e., other than a aromatic amino acid or a charged amino acid), usually L, M, V, S or T, more usually L, M, S or V; and X² and X³ independently can be any amino acid, though usually an aliphatic amino acid, a polar, uncharged amino acid, or a sulfur containing amino acid (i.e., other than an aromatic amino acid or a charged amino acid), usually S, T, A, V, G or C, more usually S, T, A, V or G.

Thus, the present disclosure provides an antibody, where the Ig light chain polypeptide of the antibody includes a constant region amino acid sequence modified to provide a tag having at least 5 amino acids and having the formula X¹Z¹X²Z²X³Z³, where Z¹ is cysteine or serine; Z² is a proline or alanine residue; Z³ is an aliphatic amino acid or a basic amino acid; X¹ is present or absent and, when present, is any amino acid; X² and X³ are each independently any amino acid, and where the Ig light chain polypeptide includes a light chain constant region containing one or more, e.g., two or more, or 3 or more, of the amino acid sequences set forth in SEQ ID NOs:46-82, shown in Table 1, as described further below.

It should be noted that, following action of an FGE on the sulfatase motif, Z¹ is oxidized to generate a formylglycine (fGly) residue. Furthermore, following both FGE-mediated conversion and reaction with a reactive partner comprising a moiety of interest (i.e., a payload, e.g., drug, detectable label, water soluble polymer, polypeptide, etc.), fGly position at Z¹ in the formula above is covalently bound to the payload.

The sulfatase motif of the tag is generally selected so as to be capable of conversion by a selected FGE, e.g., an FGE present in a host cell in which the antibody of the present disclosure is expressed or an FGE which is to be contacted with the antibody of the present disclosure in a cell-free, in vitro method.

Selection of tags and an FGE that provide for conversion of a tag to include an fGly in the target antibody containing a tagged Ig light chain polypeptide can be readily accomplished in light of information available in the art. In general, sulfatase motifs susceptible to conversion by a eukaryotic FGE contain a cysteine and a proline (i.e., a cysteine and proline at Z¹ and Z², respectively, in Formula I above (e.g., X¹CX²PX³Z³) and are modified by the “SUMF1-type” FGE (Cosma et al. Cell 2003, 113, (4), 445-56; Dierks et al. Cell 2003, 113, (4), 435-44). Sulfatase motifs susceptible to conversion by a prokaryotic FGE contain either a cysteine or a serine, and a proline in the sulfatase motif (i.e., a cysteine or serine at Z¹, and a proline at Z², respectively, in Formula I above (e.g., X¹(C/S)X²PX³Z³) are modified either by the “SUMF1-type” FGE or the “AtsB-type” FGE, respectively (Szameit et al. J Biol Chem 1999, 274, (22), 15375-81). Other sulfatase motifs susceptible to conversion by a prokaryotic FGE contain either a cysteine or a serine, and either a proline or an alanine in the sulfatase motif (i.e., a cysteine or serine at Z¹, and a proline or alanine at Z₂, respectively, in Formula I or II above (e.g., X¹CX²PX³R; X¹SX²PX²R; X¹CX²AX³R; X¹SX²AX³R; CX¹PX²R; SX¹PX²R; CX¹AX²R; SX¹AX²R, X¹CX²PX³Z³; X¹SX²PX²Z³; X¹CX²AX³Z³; X¹SX²AX³Z³; CX¹PX²Z³; SX¹PX²Z³; CX¹AX²Z³; SX¹AX²Z³), and are susceptible to modification by, for example, can be modified by an FGE of a Firmicutes (e.g., Clostridium perfringens) (see Berteau et al. J. Biol. Chem. 2006; 281:22464-22470) or an FGE of Mycobacterium tuberculosis.

Therefore, for example, where the FGE is a eukaryotic FGE (e.g., a mammalian FGE, including a human FGE), the sulfatase motif is usually of the formula: X¹CX²PX³Z³, where X¹ may be present or absent and, when present, can be any amino acid, though usually an aliphatic amino acid, a sulfur-containing amino acid, or a polar, uncharged amino acid, (i.e., other than a aromatic amino acid or a charged amino acid), usually L, M, S or V; X² and X³ independently can be any amino acid, though usually an aliphatic amino acid, a sulfur-containing amino acid, or a polar, uncharged amino acid, (i.e., other than a aromatic amino acid or a charged amino acid), usually S, T, A, V, G, or C, more usually S, T, A, V or G; and Z³ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), usually A, G, L, V, or I.

Specific examples of sulfatase motifs include LCTPSR (SEQ ID NO:158), MCTPSR (SEQ ID NO:160), VCTPSR (SEQ ID NO:161), LCSPSR (SEQ ID NO:162), LCAPSR (SEQ ID NO:163), LCVPSR (SEQ ID NO:164), LCGPSR (SEQ ID NO:165), ICTPAR (SEQ ID NO:166), LCTPSK (SEQ ID NO:167), MCTPSK (SEQ ID NO:168), VCTPSK (SEQ ID NO:169), LCSPSK (SEQ ID NO:170), LCAPSK (SEQ ID NO:171), LCVPSK (SEQ ID NO:172), LCGPSK (SEQ ID NO:173), LCTPSA (SEQ ID NO:174), ICTPAA (SEQ ID NO:175), MCTPSA (SEQ ID NO:176), VCTPSA (SEQ ID NO:177), LCSPSA (SEQ ID NO:178), LCAPSA (SEQ ID NO:179), LCVPSA (SEQ ID NO:180), LCGPSA (SEQ ID NO:181), LSTPSR (SEQ ID NO:159), MSTPSR (SEQ ID NO:182), VSTPSR (SEQ ID NO:183), LSSPSR (SEQ ID NO:184), LSAPSR (SEQ ID NO:185), LSVPSR (SEQ ID NO:186), LSGPSR (SEQ ID NO:187), ISTPAR (SEQ ID NO:188), LSTPSK (SEQ ID NO:189), MSTPSK (SEQ ID NO:190), VSTPSK (SEQ ID NO:191), LSSPSK (SEQ ID NO:192), LSAPSK (SEQ ID NO:193), LSVPSK (SEQ ID NO:194), LSGPSK (SEQ ID NO:195), LSTPSA (SEQ ID NO:196), ISTPAA (SEQ ID NO:197), MSTPSA (SEQ ID NO:198), VSTPSA (SEQ ID NO:199), LSSPSA (SEQ ID NO:200), LSAPSA (SEQ ID NO:201), LSVPSA (SEQ ID NO:202), and LSGPSA (SEQ ID NO:203). Other specific sulfatase motifs are readily apparent from the disclosure provided herein.

Antibodies Containing a Tagged Immunoglobulin Light Chain Polypeptide

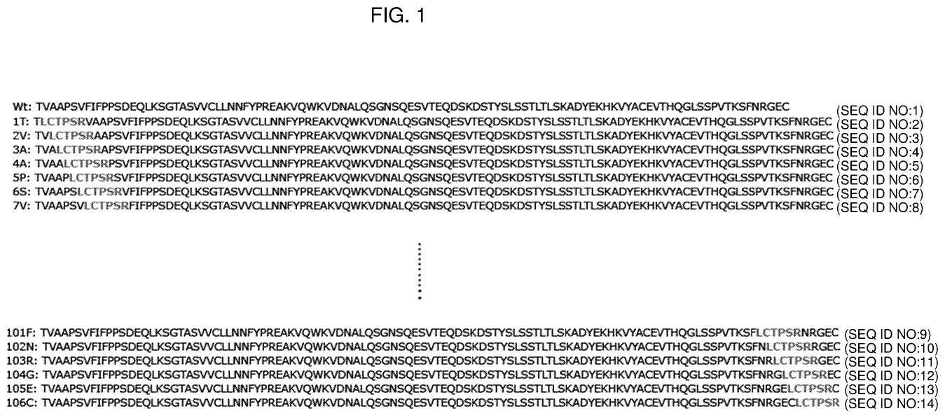

An antibody of the present disclosure contains a tag, as described above, in the amino acid sequence of an Ig light chain polypeptide constant region, where the tag is positioned between two consecutive amino acids in the constant region of a corresponding parent Ig light chain polypeptide, e.g., the Ig light chain polypeptide without the tag in the constant region. In other words, the amino acid sequence of the present tag may be present in an Ig light chain polypeptide such that the tag amino acid sequence is directly flanked N-terminally by a first flanking sequence identical to a first contiguous sequence in a corresponding parent Ig light chain constant region, and C-terminally directly flanked by a second flanking sequence identical to a second contiguous sequence in the corresponding parent Ig light chain constant region, where the first contiguous sequence is more N-terminal to the second contiguous sequence in the parent Ig light chain constant region amino acid sequence, and the first and second contiguous sequences are contiguous in the parent Ig light chain constant region amino acid sequence (see also, e.g., FIG. 1).

The parent light chain polypeptide may be an Ig kappa light chain polypeptide, having an Ig constant region amino acid sequence, e.g., as shown in FIG. 1. Thus, in some cases, the Ig light chain polypeptide is a human Ig light chain polypeptide. In some cases, the Ig light chain constant region is a human Ig light chain constant region. In some cases, the parent Ig light chain polypeptide includes a constant region amino acid sequence 75% or more, e.g., 80% or more, 85% or more, 90% or more, 95% or more, 97% or more, and up to 100% identical to the amino acid sequence set forth in SEQ ID NO:1. Thus, an antibody of the present disclosure, containing a tag, may include an Ig light chain derived from a parent Ig light chain polypeptide that is based on an Ig kappa light chain polypeptide, where the antibody contains an Ig light chain polypeptide that includes a constant region amino acid sequence, exclusive of any tags, that is 75% or more, e.g., 80% or more, 85% or more, 90% or more, 95% or more, 97% or more, and up to 100% identical to the amino acid sequence set forth in SEQ ID NO:1.

The present disclosure contemplates an antibody that includes an Ig light chain based on any suitable allotype, e.g., human allotype, of Ig kappa light chain. The Ig kappa light chain allotypes of interest include, without limitation, Km1 (having V at position 45 and L at position 83 of SEQ ID NO:1); Km1,2 (having L at position 83 of SEQ ID NO:1); and Km3 (corresponding to SEQ ID NO:1). Thus in some cases, the antibody contains an Ig light chain polypeptide that includes a constant region amino acid sequence, exclusive of any tags, that is 75% or more, e.g., 80% or more, 85% or more, 90% or more, 95% or more, 97% or more, and up to 100% identical to the Km1 allotype of Ig kappa light chain having the amino acid sequence set forth in SEQ ID NO:1, where position 45 is V and position 83 is L. In some cases, the antibody contains an Ig light chain polypeptide that includes a constant region amino acid sequence, exclusive of any tags, that is 75% or more, e.g., 80% or more, 85% or more, 90% or more, 95% or more, 97% or more, and up to 100% identical to the Km1,2 allotype of Ig kappa light chain having the amino acid sequence set forth in SEQ ID NO:1, where position 83 is L. In some cases, the antibody contains an Ig light chain polypeptide that includes a constant region amino acid sequence, exclusive of any tags, that is 75% or more, e.g., 80% or more, 85% or more, 90% or more, 95% or more, 97% or more, and up to 100% identical to the Km3 allotype of Ig kappa light chain having the amino acid sequence set forth in SEQ ID NO:1.

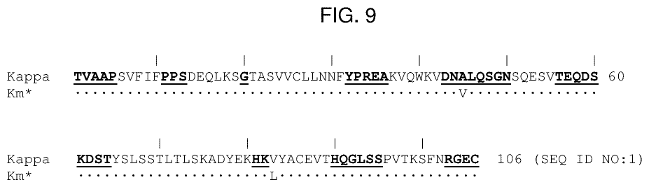

In some cases, the tag is positioned within or adjacent a solvent-accessible region of the Ig light chain polypeptide, and in some cases, the tag is not positioned within or adjacent a solvent-accessible region of the Ig light chain polypeptide. Solvent accessible loop of an antibody can be identified by molecular modeling, or by comparison to a known antibody structure. The relative accessibility of amino acid residues can also be calculated using a method of DSSP (Dictionary of Secondary Structure in Proteins; Kabsch and Sander 1983 Biopolymers 22: 2577-637) and solvent accessible surface area of an amino acid may be calculated based on a 3-dimensional model of an antibody, using algorithms known in the art (e.g., Connolly, J. Appl. Cryst. 16, 548 (1983) and Lee and Richards, J. Mol. Biol. 55, 379 (1971), both of which are incorporated herein by reference). Exemplary solvent-accessible loop regions of an Ig light chain (e.g., a human kappa light chain) include: 1) TVAAP (SEQ ID NO:464); 2) PPS; 3) Gly (see, e.g., Gly at position 20 of the human kappa light chain sequence set forth in SEQ ID NO:1, depicted in FIG. 9); 4) YPREA (SEQ ID NO:465); 5) PREA (SEQ ID NO:466); 6) DNALQSGN (SEQ ID NO:467); 7) TEQDSKDST (SEQ ID NO:468); 8) HK; 9) HQGLSS (SEQ ID NO:469); and 10) RGEC (SEQ ID NO:470), as shown in FIG. 9.

The tag of the present disclosure is positioned in the constant region of an Ig light chain polypeptide, e.g., Ig kappa light chain polypeptide, of an antibody so as to provide for an antibody having desirable properties that meet one or more threshold criteria when the antibody includes the tagged, or aldehyde tagged, Ig light chain polypeptide, as described above. An antibody of the present disclosure provides for an antibody titer of about 5 mg/L or greater, and, when the tag is converted, provides for a conjugation efficiency, represented by the average molar ratio of payload to antibody (PAR, e.g., drug-to-antibody ratio (DAR)), of about 0.5 or greater. Thus, the antibody may include a tag in an Ig light chain polypeptide, where the tag contains an amino acid sequence of the formula X¹Z¹X²Z²X³Z³, where X¹, X², X³, Z¹, Z² and Z³ are as described above, and where the Ig light chain polypeptide includes a constant region containing any one or more (e.g., two or more, or three or more) of the amino acid sequences set forth in SEQ ID NOs:46-82, shown in Table 1. In some embodiments, Z³ is arginine. In some embodiments, X¹ is glycine, leucine, isoleucine, methionine, histidine, tyrosine, valine, serine, cysteine or threonine. In some embodiments, X² and X³ are each independently serine, threonine, alanine, valine, glycine or cysteine. In some embodiments, the tag includes the amino acid sequence LCTPSR (SEQ ID NO:158). In certain embodiments, the Ig light chain polypeptide includes a constant region containing any one or more (e.g., two or more, including three or more) of the amino acid sequences set forth in SEQ ID NOs:83-119, shown in Table 2.

TABLE 1

| | | SEQ |
|---|---|---|
| | | ID |
| Label | Sequence | NO: |
| 1T | TX¹Z¹X²Z²X³Z³VA | 46 |
| 2V | TVX¹Z¹X²Z²X³Z³AA | 47 |
| 3A | VAX¹Z¹X²Z²X³Z³AP | 48 |
| 4A | AAX¹Z¹X²Z²X³Z³PSV | 49 |
| 5P | APX¹Z¹X²Z²X³Z³SVF | 50 |
| 6S | PSX¹Z¹X²Z²X³Z³VF | 51 |
| 19S | KSX¹Z¹X²Z²X³Z³GT | 52 |
| 20G | KSGX¹Z¹X²Z²X³Z³TA | 53 |
| 21T | GTX¹Z¹X²Z²X³Z³AS | 54 |
| 22A | TAX¹Z¹X²Z²X³Z³SVV | 55 |
| 29N | LNX¹Z¹X²Z²X³Z³NF | 56 |
| 30N | NNX¹Z¹X²Z²X³Z³FY | 57 |
| 31F | NFX¹Z¹X²Z²X³Z³YP | 58 |
| 32Y | FYX¹Z¹X²Z²X³Z³PR | 59 |
| 42V | WKVX¹Z¹X²Z²X³Z³DN | 60 |
| 43D | VDX¹Z¹X²Z²X³Z³N[A/V] | 61 |
| 45A | N[A/V]X¹Z¹X²Z²X³Z³LQ | 62 |
| 46L | [A/V]LX¹Z¹X²Z²X³Z³QS | 63 |
| 47Q | LQX¹Z¹X²Z²X³Z³SGN | 64 |
| 48S | QSX¹Z¹X²Z²X³Z³GN | 65 |
| 49G | QSGX¹Z¹X²Z²X³Z³NS | 66 |
| 50N | GNX¹Z¹X²Z²X³Z³SQ | 67 |
| 51S | NSX¹Z¹X²Z²X³Z³QE | 68 |
| 52Q | SQX¹Z¹X²Z²X³Z³ES | 69 |
| 60S | QDSX¹Z¹X²Z²X³Z³KD | 70 |
| 62D | KDX¹Z¹X²Z²X³Z³STY | 71 |
| 63S | KDSX¹Z¹X²Z²X³Z³TY | 72 |
| 64T | DSTX¹Z¹X²Z²X³Z³YS | 73 |
| 65Y | TYX¹Z¹X²Z²X³Z³SL | 74 |
| 89T | EVTX¹Z¹X²Z²X³Z³HQ | 75 |
| 90H | THX¹Z¹X²Z²X³Z³QG | 76 |
| 91Q | HQX¹Z¹X²Z²X³Z³GL | 77 |
| 92G | QGX¹Z¹X²Z²X³Z³LSSP | 78 |
| 93L | GLX¹Z¹X²Z²X³Z³SS | 79 |
| 94S | GLSX¹Z¹X²Z²X³Z³SP | 80 |
| 95S | GLSSX¹Z¹X²Z²X³Z³PV | 81 |
| 96P | SPX¹Z¹X²Z²X³Z³VT | 82 |

([*/*]denotes alternative amino acids (or amino acid sequences) chosen from the amino acid residues (or sequences) separated by “/”.)

TABLE 2

| | | SEQ |
|---|---|---|
| | | ID |
| ID | Sequence | NO: |
| 1T | TLCTPSRVA | 83 |
| 2V | TVLCTPSRAA | 84 |
| 3A | VALCTPSRAP | 85 |
| 4A | AALCTPSRPSV | 86 |
| 5P | APLCTPSRSVF | 87 |
| 6S | PSLCTPSRVF | 88 |
| 19S | KSLCTPSRGT | 89 |
| 20G | KSGLCTPSRTA | 90 |
| 21T | GTLCTPSRAS | 91 |
| 22A | TALCTPSRSVV | 92 |
| 29N | LNLCTPSRNF | 93 |
| 30N | NNLCTPSRFY | 94 |
| 31F | NFLCTPSRYP | 95 |
| 32Y | FYLCTPSRPR | 96 |
| 42V | WKVLCTPSRDN | 97 |
| 43D | VDLCTPSRN[A/V] | 98 |
| 45A | N[A/V]LCTPSRLQ | 99 |
| 46L | [A/V]LLCTPSRQS | 100 |
| 47Q | LQLCTPSRSGN | 101 |
| 48S | QSLCTPSRGN | 102 |
| 49G | QSGLCTPSRNS | 103 |
| 50N | GNLCTPSRSQ | 104 |
| 51S | NSLCTPSRQE | 105 |
| 52Q | SQLCTPSRES | 106 |
| 60S | QDSLCTPSRKD | 107 |
| 62D | KDLCTPSRSTY | 108 |
| 63S | KDSLCTPSRTY | 109 |
| 64T | DSTLCTPSRYS | 110 |
| 65Y | TYLCTPSRSL | 111 |
| 89T | EVTLCTPSRHQ | 112 |
| 90H | THLCTPSRQG | 113 |
| 91Q | HQLCTPSRGL | 114 |
| 92G | QGLCTPSRLSSP | 115 |
| 93L | GLLCTPSRSS | 116 |
| 94S | GLSLCTPSRSP | 117 |
| 95S | GLSSLCTPSRPV | 118 |
| 96P | SPLCTPSRVT | 119 |

([*/*] denotes alternative amino acids (or amino acid sequences) chosen from the amino acid residues (or sequences) separated by “/”.)

As described above, the tag may be positioned between two consecutive amino acids in the constant region of a corresponding parent Ig light chain polypeptide, e.g., the Ig light chain polypeptide without the tag in the constant region. Thus, the position of the tag in the Ig light chain amino acid sequence may be defined by the position of the most C-terminal amino acid of the amino acid sequence flanking the tag at its N-terminal end. In some embodiments, the tag is positioned adjacent and C-terminal to an amino acid residue, of an Ig kappa light chain polypeptide, corresponding to one or more (e.g., two or more, including three or more) of residues 1, 2, 3, 4, 5, 6, 19, 20, 21, 22, 29, 30, 31, 32, 42, 43, 45, 46, 47, 48, 49, 50, 51, 52, 60, 62, 63, 64, 65, 89, 90, 91, 92, 93, 94, 95, or 96 of SEQ ID NO:1.

The parent Ig light chain polypeptide may be modified to insert the tag in the amino acid sequence of the constant region such that an antibody that includes the tag in its Ig light chain polypeptide achieves an antibody titer of about 5 mg/L or greater, and/or a conjugation efficiency expressed by the average amount of conjugated moieties (e.g., drugs) relative to the total amount of antibody (e.g., DAR, where the payload is a drug) of about 0.5 or greater. Insertion sites of interest in an Ig light chain polypeptide include the position immediately C-terminal to an amino acid residue, of an Ig kappa light chain polypeptide, corresponding to one or more (e.g., two or more, including three or more) of positions 1, 2, 3, 4, 5, 6, 19, 20, 21, 22, 29, 30, 31, 32, 42, 43, 45, 46, 47, 48, 49, 50, 51, 52, 60, 62, 63, 64, 65, 89, 90, 91, 92, 93, 94, 95, or 96, of SEQ ID NO:1.

In certain embodiments, an antibody of the present disclosure provides for an antibody titer of about 5 mg/L or greater, and, when the tag is converted, a conjugation efficiency (e.g., DAR) of about 1.3 or greater. In some embodiments, the antibody includes a tagged Ig light chain polypeptide, where the tag contains an amino acid sequence of the formula X¹Z¹X²Z²X³Z³, where X¹, X², X³, Z¹, Z² and Z³ are as described above, and where the Ig light chain polypeptide includes a constant region containing any one or more (e.g., two or more, including three or more) of the amino acid sequences set forth in SEQ ID NOs:47, 49, 50, 53-55, 63, 65-68, 74, 77, 79, 81, and 82, of Table 1. In some embodiments, Z³ is arginine. In some embodiments, X¹ is glycine, leucine, isoleucine, methionine, histidine, tyrosine, valine, serine, cysteine or threonine. In some embodiments, X² and X³ are each independently serine, threonine, alanine, valine, glycine or cysteine. In some embodiments, the tag includes the amino acid sequence LCTPSR (SEQ ID NO:158). In certain embodiments, the Ig light chain polypeptide includes a constant region containing any one or more (e.g., two or more, including three or more) of the amino acid sequences set forth in SEQ ID NOs:84, 86, 87, 90-92, 100, 102-105, 111, 114, 116, 118 and 119, of Table 2.

The tag may be positioned between two consecutive amino acids in the constant region of a corresponding parent Ig light chain polypeptide, e.g., the Ig light chain polypeptide without the tag in the constant region. Thus, the position of the tag in the Ig light chain amino acid sequence may be defined by the position of the most C-terminal amino acid of the amino acid sequence flanking the tag at its N-terminal end. In some embodiments, the tag is positioned adjacent and C-terminal to an amino acid residue, of an Ig kappa light chain polypeptide, corresponding to one or more (e.g., two or more, including three or more) of residues 2, 4, 5, 20, 21, 22, 46, 48, 49, 50, 51, 65, 91, 93, 95, or 96, of SEQ ID NO:1.

In some cases, the parent Ig light chain polypeptide is modified to insert the tag in the amino acid sequence of the constant region such that an antibody that includes the tag in its Ig light chain polypeptide provides for an antibody titer of about 15 mg/L or greater, and, when the tag is converted, provides for a conjugation efficiency (e.g., DAR) of about 1.3 or greater. Insertion sites of interest in an Ig light chain polypeptide include the position immediately C-terminal to an amino acid residue of an Ig kappa light chain polypeptide corresponding to one or more (e.g., two or more, including three or more) of positions 2, 4, 5, 20, 21, 22, 46, 48, 49, 50, 51, 65, 91, 93, 95, or 96, of SEQ ID NO:1.

In some embodiments, the tagged Ig light chain polypeptide may include two or more, such as three or more, tags in the constant region amino acid sequence. Where the Ig light chain polypeptide includes two or more, such as three or more, tags in the constant region amino acid sequence, the position of the tags may be any two or more, such as three or more sites adjacent and C-terminal to an amino acid residue, of an Ig kappa light chain polypeptide, corresponding to one or more (e.g., two or more, including three or more) of residues 2, 4, 5, 20, 21, 22, 46, 48, 49, 50, 51, 65, 91, 93, 95, or 96, of SEQ ID NO:1, provided that there is a sufficient number of amino acid residues corresponding to the parent Ig polypeptide amino acid sequence between any two tags such that the tags do not overlap with each other.

An antibody of the present disclosure generally includes a tagged Ig light chain polypeptide constant region, as described herein, and a variable region (V_{L}), and includes an Ig heavy chain having a constant region (C_{H}1, H, C_{H}2, C_{H}3 regions) and a variable region (V_{H}), where the antibody binds an antigen. In other words, the tagged Ig light chain polypeptide forms an antigen-binding antibody when suitably combined with an Ig heavy chain polypeptide. The Ig heavy chain constant region can include heavy chain constant region sequences of an IgA, IgM, IgD, IgE, IgG₁, IgG₂, IgG₃, or IgG₄ isotype heavy chain or any allotypic variant of same, e.g., human heavy chain constant region sequences or mouse heavy chain constant region sequences, a hybrid heavy chain constant region, a synthetic heavy chain constant region, or a consensus heavy chain constant region sequence, etc. The Ig heavy chain can include one or more modifications, e.g., glycosylation, and the like.

In some embodiments, the present antibody includes an Ig heavy chain that does not include a tag, e.g., does not include a tag in the heavy chain constant region. In some embodiments, the antibody includes an Ig heavy chain having one or more, e.g., two or more, including three or more tags, i.e., a tag containing a sulfatase motif, where the tag is positioned within or adjacent a solvent-accessible loop region of the Ig heavy chain constant region. Any suitable Ig heavy chain polypeptides with a tag in the constant region may be used. Suitable Ig heavy chain polypeptides with a tag (i.e., an FGE substrate motif) in the constant region are described in, e.g., US20120183566, which is incorporated herein by reference.

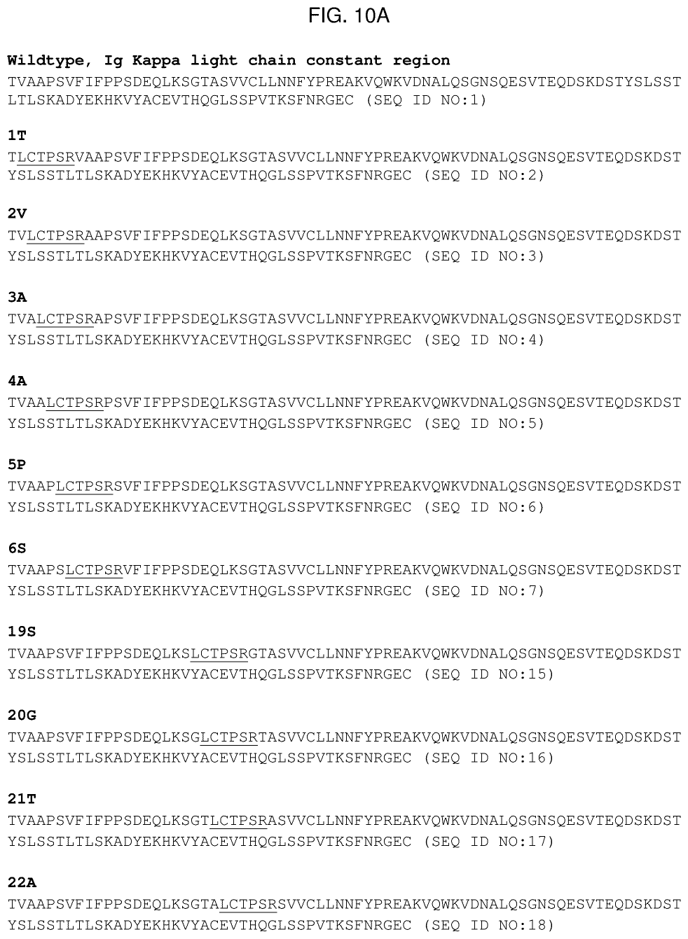

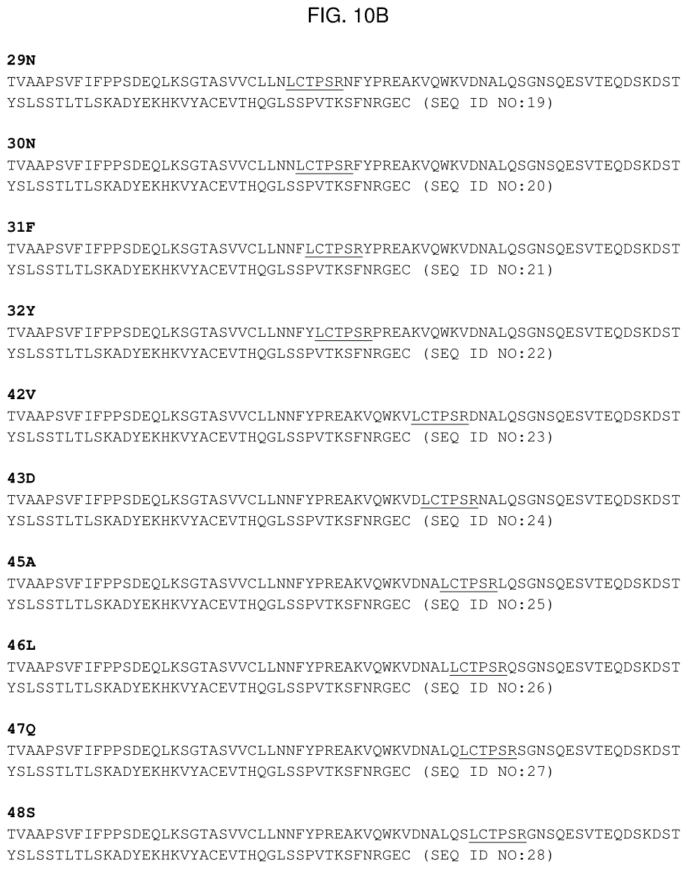

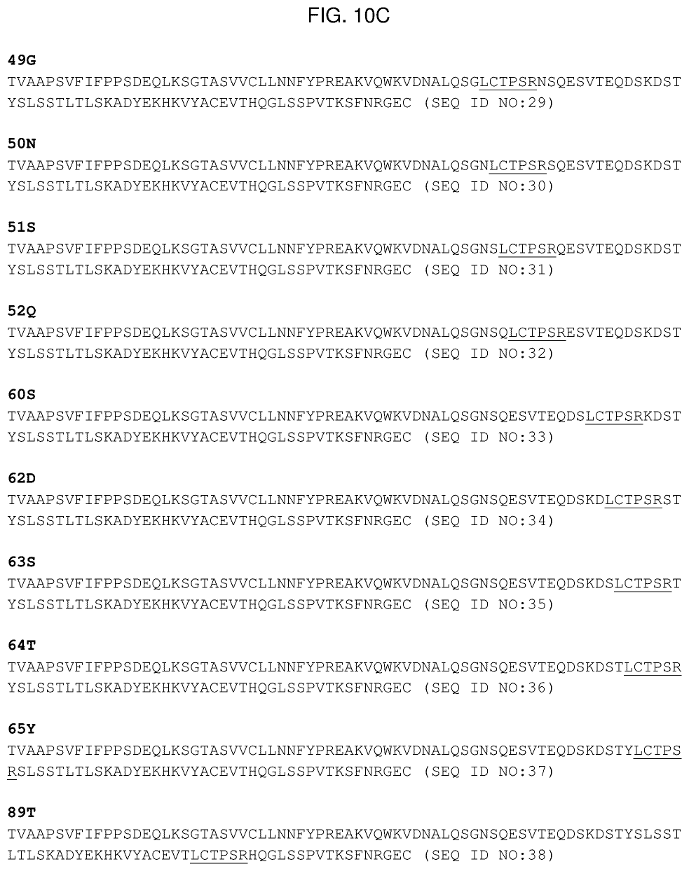

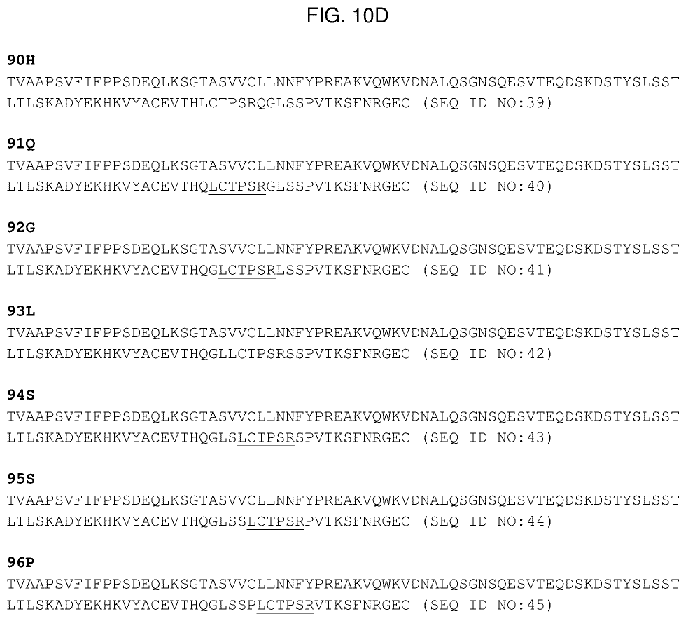

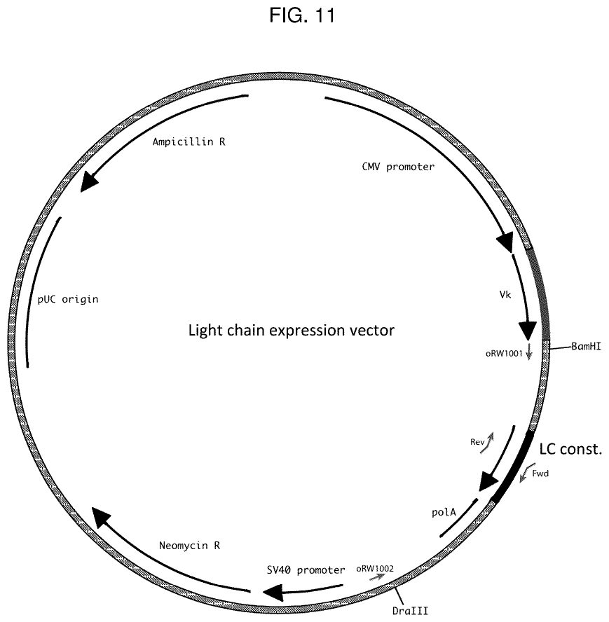

In certain embodiments, an antibody of the present disclosure includes an Ig light chain polypeptide containing an amino acid sequence at least 90%, e.g., at least 95%, at least 97%, at least 99%, including 100% identical to any one of the sequences set forth in SEQ ID NOs:2-7 and 15-45, shown in FIGS. 10A-10D, where the amino acid sequence includes the tag, i.e., the FGE substrate motif: LCTPSR (SEQ ID NO:158). The antibody having a tagged Ig light chain polypeptide may exhibit an antibody titer of about 5 mg/L or greater, e.g., about 10 mg/L or greater, about 15 mg/L or greater, about 20 mg/L or greater, or about 30 mg/L or greater, and, when the tag is converted, a conjugation efficiency (e.g., DAR) of, in some cases, about 0.5 or greater, e.g., about 1.0 or greater, or about 1.3 or greater. The Ig light chain polypeptide may include a variable region (V_{L}), and the antibody may include an Ig heavy chain having a constant region (e.g., an Fc region) and a variable region (V_{H}). The Ig heavy chain constant region can include heavy chain constant region sequences of any suitable isotype (e.g., IgA, IgM, IgD, IgE, IgG₁, IgG₂, IgG₃, or IgG₄), allotypic variant of same; human, mouse, hybrid, synthetic, or consensus heavy chain constant region sequences; modified (e.g., glycosylated) heavy chain constant region, etc. In some cases, the Ig heavy chain constant region includes one or more tags.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 1 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:2 (“1T” in FIG. 10A), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tag in its Ig light chain polypeptide may exhibit an antibody titer of about 5 mg/L or greater, e.g., about 10 mg/L or greater, about 15 mg/L or greater, about 20 mg/L or greater, including about 30 mg/L or greater, and, when the tag is converted, a conjugation efficiency (e.g., DAR) of about 0.5 or greater, including about 1.0 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 2 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:3 (“2V” in FIG. 10A), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, including about 20 mg/L or greater, and, when the tag is converted, a conjugation efficiency (e.g., DAR) of about 0.5 or greater, e.g., about 1.0 or greater, about 1.3 or greater, including about 1.6 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 3 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:4 (“3A” in FIG. 10A), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, about 20 mg/L, including about 30 mg/L or greater, and, when the tag is converted, a conjugation efficiency (e.g., DAR) of about 0.5 or greater, including about 1.0 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 4 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:5 (“4A” in FIG. 10A), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, including about 20 mg/L or greater, and, when the tag is converted, a conjugation efficiency (e.g., DAR) of about 0.5 or greater, e.g., about 1.0 or greater, including about 1.3 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 5 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:6 (“5P” in FIG. 10A), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, including about 15 mg/L, and, when the tag is converted, a conjugation efficiency (e.g., DAR) of about 0.5 or greater, e.g., about 1.0 or greater, including about 1.3 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³, inserted adjacent and C-terminal to the amino acid residue at position 6 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:7 (“6S” in FIG. 10A), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, including about 20 mg/L or greater, and, when the tag is converted, a conjugation efficiency (e.g., DAR) of about 0.5 or greater, including about 1.0 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 19 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:15 (“19S” in FIG. 10A), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, including about 20 mg/L or greater, and, when the tag is converted, a conjugation efficiency (e.g., DAR) of about 0.5 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 20 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:16 (“20G” in FIG. 10A), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, including about 20 mg/L or greater, and, when the tag is converted, a conjugation efficiency (e.g., DAR) of about 0.5 or greater, e.g., about 1.0 or greater, including about 1.3 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 21 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:17 (“21T” in FIG. 10A), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, including about 20 mg/L or greater, and, when the tag is converted, a conjugation efficiency (e.g., DAR) of about 0.5 or greater, e.g., about 1.0 or greater, about 1.3 or greater, including about 1.6 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 22 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:18 (“22A” in FIG. 10A), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, including about 20 mg/L or greater, and, when the tag is converted, a conjugation efficiency (e.g., DAR) of about 0.5 or greater, e.g., about 1.0 or greater, about 1.3 or greater, including about 1.6 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 29 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:19 (“29N” in FIG. 10B), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, including about 15 mg/L or greater, and, when the tag is converted, a conjugation efficiency (e.g., DAR) of about 0.5 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 30 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:20 (“30N” in FIG. 10B), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, including about 20 mg/L or greater, and, when the tag is converted, a conjugation efficiency (e.g., DAR) of about 0.5 or greater, including about 1.0 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 31 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:21 (“31F” in FIG. 10B), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, about 20 mg/L or greater, including 30 mg/L or greater, and, when the tag is converted, a conjugation efficiency (e.g., DAR) of about 0.5 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 32 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:22 (“32Y” in FIG. 10B), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, including about 15 mg/L or greater, and, when the tag is converted, a conjugation efficiency (e.g., DAR) of about 0.5 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 42 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:23 (“42V” in FIG. 10B), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, including about 20 mg/L or greater, and, when the tag is converted, a conjugation efficiency (e.g., DAR) of about 0.5 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 43 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:24 (“43D” in FIG. 10B), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, about 20 mg/L or greater, including 30 mg/L or greater, and, when the tag is converted, a conjugation efficiency (e.g., DAR) of about 0.5 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 45 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:25 (“45A” in FIG. 10B), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, about 20 mg/L or greater, including 30 mg/L or greater, and, when the tag is converted, a conjugation efficiency (e.g., DAR) of about 0.5 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 46 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:26 (“46L” in FIG. 10B), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, and, when the tag is converted, a conjugation efficiency (e.g., DAR) of about 0.5 or greater, e.g., about 1.0 or greater, including about 1.3 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 47 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:27 (“47Q” in FIG. 10B), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, about 20 mg/L or greater, including about 30 mg/L or greater, and, when the tag is converted, a conjugation efficiency (e.g., DAR) of about 0.5 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 48 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:28 (“48S” in FIG. 10B), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, about 20 mg/L or greater, including about 30 mg/L or greater, and, when the tag is converted, a conjugation efficiency (e.g., DAR) of about 0.5 or greater, e.g., about 1.0 or greater, including about 1.3 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 49 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:29 (“49G” in FIG. 10C), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, about 20 mg/L or greater, including about 30 mg/L or greater, and, when the tag is converted, a conjugation efficiency (e.g., DAR) of about 0.5 or greater, e.g., about 1.0 or greater, including about 1.3 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 50 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:30 (“50N” in FIG. 10C), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, about 20 mg/L or greater, including about 30 mg/L or greater, and, when the tag is converted, a conjugation efficiency (e.g., DAR) of about 0.5 or greater, e.g., about 1.0 or greater, including about 1.3 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³, inserted adjacent and C-terminal to the amino acid residue at position 51 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:31 (“51S” in FIG. 10C), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, about 20 mg/L or greater, including about 30 mg/L or greater, and, when the tag is converted, a conjugation efficiency (e.g., DAR) of about 0.5 or greater, e.g., about 1.0 or greater, including about 1.3 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 52 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:32 (“52Q” in FIG. 10C), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, including about 20 mg/L or greater, and, when the tag is converted, a conjugation efficiency (e.g., DAR) of about 0.5 or greater, including about 1.0 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 60 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:33 (“60S” in FIG. 10C), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, about 20 mg/L or greater, including about 30 mg/L or greater, and, when the tag is converted, a conjugation efficiency (e.g., DAR) of about 0.5 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 62 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:34 (“62D” in FIG. 10C), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, about 20 mg/L or greater, including about 30 mg/L or greater, and, when the tag is converted, a conjugation efficiency (e.g., DAR) of about 0.5 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 63 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:35 (“63S” in FIG. 10C), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, including about 20 mg/L or greater, and, when the tag is converted, a conjugation efficiency (e.g., DAR) of about 0.5 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 64 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:36 (“64T” in FIG. 10C), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, about 20 mg/L or greater, including about 30 mg/L or greater, and, when the tag is converted, a conjugation efficiency (e.g., DAR) of about 0.5 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 65 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:37 (“65Y” in FIG. 10C), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, including about 15 mg/L or greater, and, when the tag is converted, a conjugation efficiency (e.g., DAR) of about 0.5 or greater, e.g., about 1.0 or greater, including about 1.3 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 89 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:38 (“89T” in FIG. 10C), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, including about 20 mg/L or greater, and, when the tag is converted, a conjugation efficiency (e.g., DAR) of about 0.5 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 90 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:39 (“90H” in FIG. 10D), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, including about 15 mg/L or greater, and, when the tag is converted, a conjugation efficiency (e.g., DAR) of about 0.5 or greater, including about 1.0 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 91 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO40 (“91Q” in FIG. 10D), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, including about 15 mg/L or greater, and, when the tag is converted, a conjugation efficiency (e.g., DAR) of about 0.5 or greater, e.g., about 1.0 or greater, including about 1.3 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ adjacent and C-terminal to the amino acid residue at position 92 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:41 (“92G” in FIG. 10D), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, including about 20 mg/L or greater, and, when the tag is converted, a conjugation efficiency (e.g., DAR) of about 0.5 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 93 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:42 (“93L” in FIG. 10D), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, including about 15 mg/L or greater, and, when the tag is converted, a conjugation efficiency (e.g., DAR) of about 0.5 or greater, e.g., about 1.0 or greater, including about 1.3 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 94 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:43 (“94S” in FIG. 10D), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, including about 15 mg/L or greater, and, when the tag is converted, a conjugation efficiency (e.g., DAR) of about 0.5 or greater, including about 1.0 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 95 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:44 (“95S” in FIG. 10D), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, including about 15 mg/L or greater, and, when the tag is converted, a conjugation efficiency (e.g., DAR) of about 0.5 or greater, e.g., about 1.0 or greater, including about 1.3 or greater.

In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:1, where the Ig light chain polypeptide constant region contains a tag of the formula X¹Z¹X²Z²X³Z³ inserted adjacent and C-terminal to the amino acid residue at position 96 of SEQ ID NO:1, where X¹, X², X³, Z¹, Z² and Z³ are as described above. In some embodiments, the antibody is a modified antibody, where fGly is at Z¹. In some cases, the antibody is part of an antibody conjugate, where fGly′ is positioned at Z¹, where fGly′ is an fGly conjugated to a payload (e.g., drug). In certain embodiments, an antibody of the present disclosure includes an Ig light chain polypeptide containing a constant region amino acid sequence at least 90%, e.g., at least 95%, at least 97%, at least 99%, including 100% identical to the sequence set forth in SEQ ID NO:45 (“96P” in FIG. 10D), where the amino acid sequence includes the tag: LCTPSR (SEQ ID NO:158). In some embodiments, the antibody is a modified antibody, where the tag is converted to L(fGly)TPSR (SEQ ID NO:157). In some embodiments, the antibody is part of an antibody conjugate, where the tag is L(fGly′)TPSR (SEQ ID NO: 226), where fGly′ is an fGly conjugated to a payload (e.g., drug). The antibody having the tagged Ig light chain polypeptide may exhibit an antibody titer of about 5 mg/L or greater, e.g., about 10 mg/L or greater, about 15 mg/L or greater, including about 20 mg/L or greater, and, when the tag is converted, a conjugation efficiency (e.g., DAR) of about 0.5 or greater, e.g., about 1.0 or greater, about 1.3 or greater, including about 1.6 or greater.

The antibody of the present disclosure, or a modified form thereof (e.g., fGly-modified antibody or antibody conjugate), as described herein, may be provided in a suitable composition. Thus, the present disclosure provides a composition that includes an antibody, e.g., a plurality of members of an antibody, or a modified form thereof, of the present disclosure. The composition may include any other suitable components (e.g., buffers, stabilizers, preservatives, and the like) that are compatible with the antibody, or the modified form thereof. In some cases, the composition includes an aqueous medium, such as water, or an aqueous buffer. In some cases, the composition includes a suitable salt (i.e., is a saline solution). In some embodiments, the composition is a reconstitutable storage-stable powder or liquid composed of the present antibody and optionally any buffer or salt components. In some cases, the composition is a pharmaceutical composition, as described further below.

An antibody of the present disclosure can have any of a variety of antigen-binding specificities. The antibody can bind any of a variety of antigens, including, e.g., an antigen present on a cancer cell; an antigen present on an autoimmune cell; an antigen present on a pathogenic microorganism; an antigen present on a virus-infected cell (e.g., a human immunodeficiency virus-infected cell), e.g., CD4 or gp120; an antigen present on a diseased cell; and the like. For example, the present antibody can bind an antigen, as noted above, where the antigen is present on the surface of the cell.

For example, an antibody of the present disclosure can specifically bind an antigen present on a cancer cell. Non-limiting examples of cancer antigens that can be recognized and bound (e.g., specifically bound) by an antibody of the present disclosure include antigens present on carcinomas, prostate cancer cells, breast cancer cells, colorectal cancer cells, melanoma cells, T-cell leukemia cells, T-cell lymphoma cells, B-cell lymphoma cells, non-Hodgkin's lymphoma cells, and the like.

Non-limiting examples of antigens present on particular cancer cells include, e.g., CA125, CA15-3, CA19-9, L6, Lewis Y, Lewis X, alpha fetoprotein, CA 242, placental alkaline phosphatase, prostate specific antigen, prostatic acid phosphatase, epidermal growth factor, MAGE-1, MAGE-2, MAGE-3, MAGE-4, anti-transferrin receptor, p97, MUC1-KLH, HER2, CEA, gp100, MART1, prostate-specific antigen, human chorionic gonadotropin, IL-2 receptor, EphB2, CD19, CD20, CD22, CD52, CD33, CD38, CD40, mucin, P21, MPG, and Neu oncogene product. In some embodiments, the antigen is CD19. In other embodiments, the antigen is CD22.

Non-limiting examples of antibodies that can be modified to include a tag, as described herein, include, but are not limited to, an anti-CD19 antibody, and an anti-CD22 antibody.

FGLY-Modified Antibodies

A tagged antibody, as described above, may be modified, e.g., by oxidation of the side chain of a cysteine or serine residue in the tag into an aldehyde side chain, such that the tag is converted to a converted tag containing a 2-formylglycine residue (fGly), as described above, to generate a fGly-modified antibody. Where the Ig light chain polypeptide includes a tag containing a formylglycine generating enzyme (FGE) substrate motif of formula I, as described above, Z¹ may be modified to fGly through the action of FGE, to generate a converted tag that includes an amino acid sequence of the formula X¹(fGly)X²Z²X³Z³, where X¹, X², X³, Z², and Z³ are as described above.

The enzyme that oxidizes cysteine or serine in a sulfatase motif to fGly is referred to herein as a formylglycine generating enzyme (FGE). As discussed above, “FGE” is used herein to refer to fGly-generating enzymes that mediate conversion of a cysteine (C) of a sulfatase motif to fGly as well as fGly-generating enzymes that mediate conversion of serine (S) of a sulfatase motif to fGly. It should be noted that in general, the literature refers to fGly-generating enzymes that convert a C to fGly in a sulfatase motif as FGEs, and refers to enzymes that convert S to fGly in a sulfatase motif as Ats-B-like. However, for purposes of the present disclosure “FGE” is used generically to refer to both types of fGly-generating enzymes, with the understanding that an appropriate FGE will be selected according to the target reactive partner containing the appropriate sulfatase motif (i.e., C-containing or S-containing).

In general, the FGE used to facilitate conversion of cysteine or serine to fGly in a sulfatase motif of a tag of a target polypeptide is selected according to the sulfatase motif present in the tag. The FGE can be native to the host cell in which the tag-containing polypeptide is expressed, or the host cell can be genetically modified to express an appropriate FGE. In some embodiments it may be desired to use a sulfatase motif compatible with a human FGE (e.g., the SUMF1-type FGE, see, e.g., Cosma et al. Cell 113, 445-56 (2003); Dierks et al. Cell 113, 435-44 (2003)), and express the aldehyde tagged protein in a human cell that expresses the FGE or in a host cell, usually a mammalian cell, genetically modified to express a human FGE.

In general, an FGE for use in the methods disclosed herein can be obtained from naturally occurring sources or synthetically produced. For example, an appropriate FGE can be derived from biological sources which naturally produce an FGE or which are genetically modified to express a recombinant gene encoding an FGE. Nucleic acids encoding a number of FGEs are known in the art and readily available (see, e.g., Preusser et al. 2005 J. Biol. Chem. 280(15):14900-10 (Epub 2005 Jan. 18); Fang et al. 2004 J Biol Chem. 79(15):14570-8 (Epub 2004 Jan. 28); Landgrebe et al. Gene. 2003 Oct. 16; 316:47-56; Dierks et al. 1998 FEBS Lett. 423(1):61-5; Dierks et al. Cell. 2003 May 16; 113(4):435-44; Cosma et al. (2003 May 16) Cell 113(4):445-56; Baenziger (2003 May 16) Cell 113(4):421-2 (review); Dierks et al. Cell. 2005 May 20; 121(4):541-52; Roeser et al. (2006 Jan. 3)Proc Natl Acad Sci USA 103(1):81-6; Sardiello et al. (2005 November 1) Hum Mol Genet. 14(21):3203-17; WO 2004/072275; WO 2008/036350; U.S. Patent Publication No. 2008/0187956; and GenBank Accession No. NM_182760. Accordingly, the disclosure here provides for recombinant host cells genetically modified to express an FGE that is compatible for use with a tag of a target polypeptide. In certain embodiments, the FGE used may be a naturally occurring enzyme (may have a wild type amino acid sequence). In other embodiments, the FGE used may be non-naturally occurring, in which case it may, in certain cases, have an amino acid sequence that is at least 80% identical, at least 90% identical or at least 95% identical to that of a wild type enzyme. Because FGEs have been studied structurally and functionally and the amino acid sequences of several examples of such enzymes are available, variants that retain enzymatic activity should be readily designable.

Where a cell-free method is used to convert a sulfatase motif-containing polypeptide, an isolated FGE can be used. Any convenient protein purification procedures may be used to isolate an FGE, see, e.g., Guide to Protein Purification, (Deuthser ed.) (Academic Press, 1990). For example, a lysate may be prepared from a cell that produces a desired FGE, and purified using HPLC, exclusion chromatography, gel electrophoresis, affinity chromatography, and the like.

Any suitable method of generating a tagged antibody having a sulfatase motif in its Ig polypeptide, e.g., Ig light chain polypeptide, and converting the tag to include an fGly residue, may be used, e.g., as described in US20120183566, which is incorporated herein by reference.

Thus, the present disclosure includes an fGly-modified antibody that includes a converted tag in an fGly-modified Ig light chain polypeptide, e.g., fGly-modified Ig kappa light chain polypeptide, where the converted tag contains an amino acid sequence of the formula:

X¹(fGly)X²Z²X³Z³ (II)

where fGly is a formylglycine residue; Z² is either a proline or alanine residue (which can also be represented by (P/A)); Z³ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), usually A, G, L, V, or I; X¹ is present or absent and, when present, can be any amino acid, though usually an aliphatic amino acid, a sulfur-containing amino acid, or a polar, uncharged amino acid, (i.e., other than a aromatic amino acid or a charged amino acid), usually L, M, V, S or T, more usually L, M, S or V; and X² and X³ independently can be any amino acid, though usually an aliphatic amino acid, a polar, uncharged amino acid, or a sulfur containing amino acid (i.e., other than an aromatic amino acid or a charged amino acid), usually S, T, A, V, G or C, more usually S, T, A, V or G.

As the converted tag may be derived from an unconverted tag, e.g., through the oxidation of a cysteine or serine in the sulfatase motif of a tag via the action of FGE, the position of the converted tag may be defined by the position of the tag in the Ig light chain polypeptide, as described above. Thus in some embodiments, an fGly-modified antibody of the present disclosure includes an fGly-modified Ig light chain polypeptide that includes a converted tag having an amino acid sequence of the formula X¹Z¹X²Z²X³Z³, where X¹, X², X³, Z² and Z³ are as described above, and where Z¹ is fGly, and where the fGly-modified Ig light chain polypeptide includes a constant region containing any one or more (e.g., two or more, including three or more) of the amino acid sequences set forth in SEQ ID NOs:46-82, shown in Table 1, where Z¹ is fGly and X¹, X², X³, Z² and Z³ are as described above. In some embodiments, the converted tag contained in the fGly-modified Ig light chain polypeptide has the amino acid sequence L(fGly)TPSR (SEQ ID NO:157). In some embodiments, the fGly-modified Ig light chain polypeptide includes a constant region containing any one or more (e.g., two or more, including three or more) of the amino acid sequences set forth in SEQ ID NOs:83-119, shown in Table 2, where Cys is substituted with fGly. As described above, the antibody containing a converted tag in an Ig light chain polypeptide constant region may exhibit a conjugation efficiency, represented by the average molar ratio of payload to antibody (e.g., drug to antibody (DAR)), of about 0.5 or greater.

In some cases, the present antibody containing an converted tag in an Ig light chain polypeptide constant region provides for a conjugation efficiency, represented by the average amount of conjugated moieties (e.g., drugs) relative to the total amount of antibody, of 1.3 or greater. In such cases, the fGly-modified Ig light chain polypeptide includes a constant region containing any one or more (e.g., two or more, including three or more) of the amino acid sequences set forth in SEQ ID NOs:47, 49, 50, 53-55, 63, 65-68, 74, 77, 79, 81, and 82, of Table 1, where Z¹ is fGly and X¹, X², X³, Z² and Z³ are as described above. In some cases, where the tag is LCTPSR (SEQ ID NO:158), the fGly-modified Ig light chain polypeptide includes a constant region containing any one or more (e.g., two or more, including three or more) of the amino acid sequences set forth in SEQ ID NOs:84, 86, 87, 90-92, 100, 102-105, 111, 114, 116, 118 and 119, of Table 2, where Cys is substituted with fGly.

Antibody Conjugates

An antibody containing an fGly-modified Ig light chain polypeptide, as described above, may be modified to covalently attach a moiety of interest (i.e., a payload, e.g., drug) to the antibody in a site-specific manner, to produce an antibody conjugate. As described above, the aldehyde moiety of the fGly residue in the converted tag of an Ig light chain polypeptide provides a bioorthogonal reactive side chain with which an aldehyde-reactive group attached to a payload, e.g., a drug functionalized with an aldehyde-reactive group, can react in a chemoselective manner to form a covalent bond between the payload (e.g., drug) and the Ig light chain polypeptide via the fGly residue, to form an Ig light chain polypeptide conjugate. A payload conjugated to an antibody of the present disclosure includes any suitable moiety (e.g., drug, detectable label, water soluble polymer, polypeptide, etc.) that, prior to conjugation to an fGly-modified antibody, can be functionalized to from an aldehyde-reactive reactive partner that includes an aldehyde-reactive group attached to the payload.

Thus, the present disclosure includes an antibody conjugate that includes a modified tag in an Ig light chain polypeptide conjugate, e.g., Ig kappa light chain polypeptide conjugate, where the modified tag contains an amino acid sequence of the formula:

X¹(fGly′)X²Z²X³Z³ (III)

where fGly′ is a formylglycine residue modified with a payload (e.g., drug) covalently attached thereto; Z² is either a proline or alanine residue (which can also be represented by (P/A)); Z³ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), usually A, G, L, V, or I; X¹ is present or absent and, when present, can be any amino acid, though usually an aliphatic amino acid, a sulfur-containing amino acid, or a polar, uncharged amino acid, (i.e., other than a aromatic amino acid or a charged amino acid), usually L, M, V, S or T, more usually L, M, S or V; and X² and X³ independently can be any amino acid, though usually an aliphatic amino acid, a polar, uncharged amino acid, or a sulfur containing amino acid (i.e., other than an aromatic amino acid or a charged amino acid), usually S, T, A, V, G or C, more usually S, T, A, V or G.

As the modified tag, having a payload (e.g., drug) conjugated thereto, may be derived from a converted tag, e.g., through the reaction of a aldehyde-reactive reactive partner containing the payload (e.g., drug with the aldehyde group of fGly, the position of the modified tag may be defined by the position of the converted tag in the Ig light chain polypeptide, which in turn may be defined by the position of the tag in the Ig light chain polypeptide, as described above. Thus in some embodiments, an antibody conjugate of the present disclosure includes an Ig light chain polypeptide conjugate that includes a modified tag having an amino acid sequence of the formula X¹Z¹X²Z²X³Z³, where X¹, X², X³, Z² and Z³ are as described above, and where Z¹ is fGly′, where fGly′ is a formylglycine residue modified with a payload (e.g., drug) covalently attached thereto, and where the Ig light chain polypeptide conjugate includes a constant region containing any one of the amino acid sequences set forth in SEQ ID NOs:46-82, shown in Table 1, where Z¹ is fGly′, where fGly′ is a formylglycine residue modified with a payload (e.g., drug) covalently attached thereto, and where X¹, X², X³, Z² and Z³ are as described above. In some embodiments, the antibody conjugate includes an Ig light chain polypeptide conjugate having the amino acid sequence L(fGly′)TPSR (SEQ ID NO: 226) in the constant region. In some embodiments, the Ig light chain polypeptide conjugate includes a constant region containing any one of the amino acid sequences set forth in SEQ ID NOs:83-119, shown in Table 2, where Cys is substituted with fGly′, where fGly′ is a formylglycine residue modified with a payload (e.g., drug) covalently attached thereto.

The structure of fGly′ may vary, and may depend on the structure of the aldehyde-reactive group used to react a reactive partner containing the payload (e.g., drug) with the aldehyde side chain of the fGly residue in a converted tag of an fGly-modified Ig light chain polypeptide. fGly′ may include any suitable linkage between the Ig polypeptide backbone and the payload (e.g., drug). In some cases, the payload (e.g., drug) is covalently bound to the converted tag through the fGly, which is modified, through its reaction with the aldehyde-reactive group, to form a hydrazone, oxime, semicarbazone (e.g., thiosemicarbozone), alkyl, alkenyl, acyloxy, hydrazinyl-indolyl, hydrazinyl-imidazoyl, hydrazinyl-pyrrolyl, hydrazinyl-furanyl or a pyrazolinone-derived linkage, and derivatives of such linkages, with the payload (e.g., drug). A hydrazinyl-indolyl linkage may include, e.g., a partially unsaturated pyrazole or pyridazine ring, or a partially unsaturated pyridazine or 1,2-diazepine ring. A pyrazolinone-derived linkage may include a cyclic linkage derived from a pyrazolinone. In some cases, a hydrazinyl-substituted heteroaryl ring-derived linkage includes a cyclic linkage derived from, e.g., a hydrazinyl-substituted 5-membered heteroaryl ring compound, where one or more atoms in the ring is a heteroatom (e.g., N, O or S). The hydrazinyl-substituted heteroaryl ring-derived linkage may include a hydrazinyl-imidazoyl, hydrazinyl-pyrrolyl, or a hydrazinyl-furanyl linkage. Suitable linkages between the fGly of a converted tag and the payload are described in, e.g., US20120183566, US20140141025, and WO2014074218, each of which is incorporated herein by reference.

The payload (e.g., drug), in some cases, may be covalently bound to the fGly of a converted tag via one or more linking groups, in addition to the covalent linkage formed by a reaction between the aldehyde-reactive group and the aldehyde group of fGly of the converted tag. Thus the linking group may serve as a spacer between the payload (e.g., drug) and the covalent linkage with the modified fGly of the tag in the Ig light chain polypeptide conjugate. The linking group may be any suitable linking group. In some cases, the linking group includes polyethylene glycol (PEG); amino acids; alkyl groups, including substituted alkyl groups; a protease cleavable group; esters; acyloxy groups, including substituted acyloxy groups, etc. Suitable linking groups are described in, e.g., US20150157736, which is incorporated by reference herein. In some embodiments, the linking group includes a 4-aminopiperidine (4AP) derivative.

An antibody conjugate of the present disclosure can include: 1) Ig heavy chain constant region that is not conjugated to a payload (e.g., drug); and an Ig light chain constant region conjugated to a payload (e.g., drug) of interest; or 2) an Ig heavy chain constant region conjugated to a payload (e.g., drug); and an Ig light chain constant region conjugated to a payload (e.g., drug). A subject antibody conjugate can also include V_{H} and/or V_{L} domains.

An antibody conjugate can have any of a variety of antigen-binding specificities, as described above, including, e.g., an antigen present on a cancer cell; an antigen present on an autoimmune cell; an antigen present on a pathogenic microorganism; an antigen present on a virus-infected cell (e.g., a human immunodeficiency virus-infected cell), e.g., CD4 or gp120; an antigen present on a diseased cell; and the like. For example, an antibody conjugate can bind an antigen, as noted above, where the antigen is present on the surface of the cell. The binding specificity, affinity, etc., of the antibody conjugate may be determined by at least the light and heavy chain variable region CDR sequences and/or the light and heavy chain variable regions (including the framework regions) included in the antibody conjugate. Thus the binding specificity, affinity, etc., of the antibody conjugate typically may have substantially the same antigen binding specificity, affinity, etc., as an antibody that may not be conjugated to a payload, or be tagged, and which has at least the same light and heavy chain variable region CDR sequences and/or the same light and heavy chain variable regions as the antibody conjugate.

An antibody conjugate of the present disclosure can bind antigen with a suitable binding affinity, e.g., from about 5×10⁻⁶ M to about 10⁻⁷ M, from about 10⁻⁷ M to about 5×10⁻⁷ M, from about 5×10⁻⁷ M to about 10⁻⁸ M, from about 10⁻⁸ M to about 5×10⁻⁸ M, from about 5×10⁻⁸ M to about 10-9 M, or a binding affinity greater than 10-9 M.

As non-limiting examples, a subject antibody conjugate can bind an antigen present on a cancer cell (e.g., a tumor-specific antigen; an antigen that is over-expressed on a cancer cell; etc.), and the attached payload can be a cytotoxic compound (e.g., a cytotoxic small molecule, a cytotoxic synthetic peptide, etc.). For example, a subject antibody conjugate can be specific for CD19, where the attached payload is a cytotoxic compound (e.g., a cytotoxic small molecule, a cytotoxic synthetic peptide, etc.). As another example, a subject antibody conjugate can be specific for CD22, where the attached payload can be a cytotoxic compound (e.g., a cytotoxic small molecule, a cytotoxic synthetic peptide, etc.).

As further non-limiting examples, a subject antibody conjugate can bind an antigen present on a cell infected with a virus (e.g., where the antigen is encoded by the virus; where the antigen is expressed on a cell type that is infected by a virus; etc.), and the payload can be a viral fusion inhibitor. For example, a subject antibody conjugate can bind CD4, and the attached payload can be a viral fusion inhibitor. As another example, a subject antibody conjugate can bind gp120, and the attached payload can be a viral fusion inhibitor.

As described above, a payload conjugated to an antibody of the present disclosure includes any suitable moiety (e.g., drug, detectable label, water soluble polymer, polypeptide, etc.) that, prior to conjugation to an fGly-modified antibody, can be functionalized to from an aldehyde-reactive reactive partner that includes an aldehyde-reactive group attached to the payload.

An antibody conjugate of the present disclosure can include, as the payload (e.g., drug), any of a variety of compounds, as described above, e.g., a drug (e.g., a peptide drug, a small molecule drug, and the like), a water-soluble polymer, a detectable label, a synthetic peptide, etc. In general, the payload or payloads (e.g., drug or drugs) can provide for one or more of a wide variety of functions or features. Moieties of interest include, without limitation, detectable labels (e.g., dye labels (e.g., chromophores, fluorophores), biophysical probes (spin labels, nuclear magnetic resonance (NMR) probes), Förster Resonance Energy Transfer (FRET)-type labels (e.g., at least one member of a FRET pair, including at least one member of a fluorophore/quencher pair), Bioluminescence Resonance Energy Transfer (BRET)-type labels (e.g., at least one member of a BRET pair), immunodetectable tags (e.g., FLAG (e.g., DYKDDDDK (SEQ ID NO:249)), His(6), and the like), localization tags (e.g., to identify association of a tagged polypeptide at the tissue or molecular cell level (e.g., association with a tissue type, or particular cell membrane)), and the like); light-activated dynamic moieties (e.g., azobenzene mediated pore closing, azobenzene mediated structural changes, photodecaging recognition motifs); water soluble polymers (e.g., PEGylation); purification tags (e.g., to facilitate isolation by affinity chromatography (e.g., attachment of a FLAG epitope, e.g., DYKDDDDK (SEQ ID NO:249)); membrane localization domains (e.g., lipids or glycophosphatidylinositol (GPI)-type anchors); immobilization tags (e.g., to facilitate attachment of the polypeptide to a surface, including selective attachment); drugs (e.g., to facilitate drug targeting, e.g., through attachment of the drug to an antibody); toxins; targeted delivery moieties, (e.g., ligands for binding to a target receptor (e.g., to facilitate viral attachment, attachment of a targeting protein present on a liposome, etc.)), other molecules for delivery to the cell and which can provide for a pharmacological activity or can serve as a target for delivery of other molecules, and the like.

Also contemplated is a covalently attached payload (e.g., drug) that comprises one of a pair of binding partners (e.g., a ligand, a ligand-binding portion of a receptor, a receptor-binding portion of a ligand, etc.). For example, the payload can comprise a polypeptide that serves as a viral receptor and, upon binding with a viral envelope protein or viral capsid protein, facilitates attachment of virus to the cell surface with which the antibody conjugate is associated, e.g., is bound. Alternatively, the payload comprises an antigen that is specifically bound by an antibody (e.g., monoclonal antibody), to facilitate detection and/or separation of host cells bound to the antibody conjugate containing the payload.

Water-Soluble Polymers

In some cases, an antibody conjugate comprises a covalently linked payload that is a water-soluble polymer. A moiety of particular interest is a water-soluble polymer. A “water-soluble polymer” refers to a polymer that is soluble in water and is usually substantially non-immunogenic, and usually has an atomic molecular weight greater than about 1,000 Daltons. The methods and compositions described herein can be used to attach one or more water-soluble polymers to a tagged and converted polypeptide. Attachment of a water-soluble polymer (e.g., PEG) of a polypeptide, particularly a pharmaceutically active (therapeutic) polypeptide can be desirable as such modification can increase therapeutic index by increasing serum half-life as a result of increased proteolytic stability and/or decreased renal clearance. Additionally, attachment of one or more polymers (e.g., PEGylation) can reduce immunogenicity of protein pharmaceuticals.

In some embodiments, the water-soluble polymer has an effective hydrodynamic molecular weight of greater than about 10,000 Da, greater than about 20,000 to 500,000 Da, greater than about 40,000 Da to 300,000 Da, greater than about 50,000 Da to 70,000 Da, usually greater than about 60,000 Da. In some embodiments, the water-soluble polymer has an effective hydrodynamic molecular weight of from about 10 kDa to about 20 kDa, from about 20 kDa to about 25 kDa, from about 25 kDa to about 30 kDa, from about 30 kDa to about 50 kDa, or from about 50 kDa to about 100 kDa. By “effective hydrodynamic molecular weight” is intended the effective water-solvated size of a polymer chain as determined by aqueous-based size exclusion chromatography (SEC). When the water-soluble polymer contains polymer chains having polyalkylene oxide repeat units, such as ethylene oxide repeat units, each chain can have an atomic molecular weight of between about 200 Da and about 80,000 Da, or between about 1,500 Da and about 42,000 Da, with 2,000 to about 20,000 Da being of particular interest. Unless referred to specifically, molecular weight is intended to refer to atomic molecular weight. Linear, branched, and terminally charged water soluble polymers (e.g., PEG) are of particular interest.

Polymers useful as moieties to be conjugated to an fGly-modified antibody to form an antibody conjugate can have a wide range of molecular weights, and polymer subunits. These subunits may include a biological polymer, a synthetic polymer, or a combination thereof. Examples of such water-soluble polymers include: dextran and dextran derivatives, including dextran sulfate, P-amino cross linked dextrin, and carboxymethyl dextrin, cellulose and cellulose derivatives, including methylcellulose and carboxymethyl cellulose, starch and dextrines, and derivatives and hydroylactes of starch, polyalklyene glycol and derivatives thereof, including polyethylene glycol, methoxypolyethylene glycol, polyethylene glycol homopolymers, polypropylene glycol homopolymers, copolymers of ethylene glycol with propylene glycol, wherein said homopolymers and copolymers are unsubstituted or substituted at one end with an alkyl group, heparin and fragments of heparin, polyvinyl alcohol and polyvinyl ethyl ethers, polyvinylpyrrolidone, aspartamide, and polyoxyethylated polyols, with the dextran and dextran derivatives, dextrine and dextrine derivatives. It will be appreciated that various derivatives of the specifically recited water-soluble polymers are also contemplated.

Water-soluble polymers such as those described above are well known, particularly the polyalkylene oxide based polymers such as polyethylene glycol “PEG”. Suitable polymers include, without limitation, those containing a polyalkylene oxide, polyamide alkylene oxide, or derivatives thereof, including polyalkylene oxide and polyamide alkylene oxide comprising an ethylene oxide repeat unit of the formula —(CH₂—CH₂—O)—. Further exemplary polymers of interest include a polyamide having a molecular weight greater than about 1,000 Daltons of the formula —[C(O)—X—C(O)—NH—Y—NH]n- or —[NH—Y—NH—C(O)—X—C(O)]ₙ—, where X and Y are divalent radicals that may be the same or different and may be branched or linear, and n is a discrete integer from 2-100, usually from 2 to 50, and where either or both of X and Y comprises a biocompatible, substantially non-antigenic water-soluble repeat unit that may be linear or branched. Further exemplary water-soluble repeat units comprise an ethylene oxide of the formula —(CH₂—CH₂—O)— or —(CH₂—CH₂—O)—. The number of such water-soluble repeat units can vary significantly, with the usual number of such units being from 2 to 500, 2 to 400, 2 to 300, 2 to 200, 2 to 100, and most usually 2 to 50. An exemplary embodiment is one in which one or both of X and Y is selected from: —((CH₂)ₙ₁—(CH₂—CH₂—O)ₙ₂—(CH₂)— or —((CH₂)ₙ₁—(O—CH₂—CH₂)ₙ₂—(CH₂)ₙ₋₁—), where n1 is 1 to 6, 1 to 5, 1 to 4 and most usually 1 to 3, and where n2 is 2 to 50, 2 to 25, 2 to 15, 2 to 10, 2 to 8, and most usually 2 to 5. A further exemplary embodiment is one in which X is —(CH₂—CH₂)—, and where Y is —(CH₂—(CH₂—CH₂—O)₃—CH₂—CH₂—CH₂)— or —(CH₂—CH₂—CH₂—(O—CH₂—CH₂)₃—CH₂)—.

The polymer can include one or more spacers or linkers. Exemplary spacers or linkers include linear or branched moieties comprising one or more repeat units employed in a water-soluble polymer, diamino and or diacid units, natural or unnatural amino acids or derivatives thereof, as well as aliphatic moieties, including alkyl, aryl, heteroalkyl, heteroaryl, alkoxy, and the like, which can contain, for example, up to 18 carbon atoms or even an additional polymer chain.

The polymer moiety, or one or more of the spacers or linkers of the polymer moiety when present, may include polymer chains or units that are biostable or biodegradable. For example, polymers with repeat linkages have varying degrees of stability under physiological conditions depending on bond lability. Polymers with such bonds can be categorized by their relative rates of hydrolysis under physiological conditions based on known hydrolysis rates of low molecular weight analogs, e.g., from less stable to more stable, e.g., polyurethanes (—NH—C(O)—O—)>polyorthoesters (—O—C((OR)(R′))—O—)>polyamides (—C(O)—NH—). Similarly, the linkage systems attaching a water-soluble polymer to a target molecule may be biostable or biodegradable, e.g., from less stable to more stable: carbonate (—O—C(O)—O—)>ester (—C(O)—O—)>urethane (—NH—C(O)—O—)>orthoester (—O—C((OR)(R′))—O—)>amide (—C(O)—NH—). In general, it may be desirable to avoid use of sulfated polysaccharide, depending on the lability of the sulfate group. In addition, it may be less desirable to use polycarbonates and polyesters. These bonds are provided by way of example, and are not intended to limit the types of bonds employable in the polymer chains or linkage systems of the water-soluble polymers useful in the modified aldehyde tagged polypeptides disclosed herein.

Synthetic Peptides

In some cases, an antibody conjugate comprises a covalently linked peptide, e.g., a peptide covalently linked to fGly of a converted tag of an Ig light chain polypeptide of an antibody. Suitable peptides include, but are not limited to, cytotoxic peptides; angiogenic peptides; anti-angiogenic peptides; peptides that activate B cells; peptides that activate T cells; anti-viral peptides; peptides that inhibit viral fusion; peptides that increase production of one or more lymphocyte populations; anti-microbial peptides; growth factors; growth hormone-releasing factors; vasoactive peptides; anti-inflammatory peptides; peptides that regulate glucose metabolism; an anti-thrombotic peptide; an anti-nociceptive peptide; a vasodilator peptide; a platelet aggregation inhibitor; an analgesic; and the like.

Where the covalently attached moiety is a peptide, the peptide can be chemically synthesized to include a group reactive with a converted fGly-containing Ig polypeptide. A suitable synthetic peptide has a length of from about 5 amino acids to about 100 amino acids, or longer than 100 amino acids; e.g., a suitable peptide has a length of from about 5 amino acids (aa) to about 10 aa, from about 10 aa to about 15 aa, from about 15 aa to about 20 aa, from about 20 aa to about 25 aa, from about 25 aa to about 30 aa, from about 30 aa to about 40 aa, from about 40 aa to about 50 aa, from about 50 aa to about 60 aa, from about 60 aa to about 70 aa, from about 70 aa to about 80 aa, from about 80 aa to about 90 aa, or from about 90 aa to about 100 aa.

A peptide can be modified to contain an α-nucleophile-containing moiety (e.g., an aminooxy or hydrazide moiety), e.g., can be reacted with the fGly-containing Ig polypeptide to yield a conjugate in which the aldehyde-tagged Ig polypeptide and peptide are linked by a hydrazone or oxime bond, respectively. Exemplary methods of synthesizing a peptide, such that the synthetic peptide comprising a reactive group reactive with a converted aldehyde tag, are described above.

Suitable peptides include, but are not limited to, hLF-11 (an 11-amino acid N-terminal fragment of lactoferrin), an anti-microbial peptide; granulysin, an anti-microbial peptide; Plectasin (NZ2114; SAR 215500), an anti-microbial peptide; viral fusion inhibitors such as Fuzeon (enfuvirtide), TRI-1249 (T-1249; see, e.g., Matos et al. (2010) PLoS One 5:e9830), TRI-2635 (T-2635; see, e.g., Eggink et al. (2009) J. Biol. Chem. 284:26941), T651, and TRI-1144; C5a receptor inhibitors such as PMX-53, JPE-1375, and JSM-7717; POT-4, a human complement factor C3 inhibitor; Pancreate (an INGAP derivative sequence, a HIP-human proislet protein); somatostatin; a somatostatin analog such as DEBIO 8609 (Sanvar), octreotide, octreotide (C2L), octreotide QLT, octreotide LAR, Sandostatin LAR, SomaLAR, Somatuline (lanreotide), see, e.g., Deghenghi et al. (2001) Endocrine 14:29; TH9507 (Tesamorelin, a growth hormone-releasing factor); POL7080 (a protegrin analog, an anti-microbial peptide); relaxin; a corticotropin releasing factor agonist such as urotensin, sauvagine, and the like; a heat shock protein derivative such as DiaPep277; a human immunodeficiency virus entry inhibitor; a heat shock protein-20 mimic such as AZX100; a thrombin receptor activating peptide such as TP508 (Chrysalin); a urocortin 2 mimic (e.g., a CRF2 agonist) such as urocortin-2; an immune activator such as Zadaxin (thymalfasin; thymosin-α1), see, e.g., Sjogren (2004) J. Gastroenterol. Hepatol. 19:569; a hepatitis C virus (HCV) entry inhibitorE2 peptide such as HCV3; an atrial natriuretic peptide such as HANP (Sun 4936; carperitide); an annexin peptide; a defensin (anti-microbial peptide) such as hBD2-4; a defensin (anti-microbial peptide) such as hBD-3; a defensin (anti-microbial peptide) such as PMX-30063; a histatin (anti-microbial peptide) such as histatin-3, histatin-5, histatin-6, and histatin-9; a histatin (anti-microbial peptide) such as PAC-113; an indolicidin (anti-microbial peptide) such as MX-594AN (Omniganin; CLS001); an indolicidin (anti-microbial peptide) such as Omnigard (MBI-226; CPI-226); an anti-microbial peptide such as an insect cecropin; an anti-microbial peptide such as a lactoferrin (talactoferrin); an LL-37/cathelicidin derivative (an anti-microbial peptide) such as P60.4 (OP-145); a magainin (an anti-microbial peptide) such as Pexiganan (MSI-78; Suponex); a protegrin (an anti-microbial peptide) such as IB-367 (Iseganan); an agan peptide; a beta-natriuretic peptide such as Natrecor, or Noratak (Nesiritide), or ularitide; bivalarudin (Angiomax), a thrombin inhibitor; a C peptide derivative; a calcitonin such as Miacalcin (Fortical); an enkephalin derivative; an erythropoiesis-stimulating peptide such as Hematide; a gap junction modulator such as Danegaptide (ZP1609); a gastrin-releasing peptide; a ghrelin; a glucagon-like peptide; a glucagon-like peptide-2 analog such as ZP1846 or ZP1848; a glucosaminyl muramyl dipeptide such as GMDP; a glycopeptide antibiotic such as Oritavancin; a teicoplanin derivative such as Dalbavancin; a gonadotropin releasing hormone (GnRH) such as Zoladex (Lupon) or Triptorelin; a histone deacetylase (HDAC) inhibitor depsipeptide such as PM02734 (Irvalec); an integrin such as eptifibatide; an insulin analog such as Humulog; a kahalalide depsipeptide such as PM02734; a kallikrein inhibitor such as Kalbitor (ecallantide); an antibiotic such as Telavancin; a lipopeptide such as Cubicin or MX-2401; a lutenizing hormone releasing hormone (LHRH) such as goserelin; an LHRH synthetic decapeptide agonist analog such as Treistar (triptorelin pamoate); an LHRH such as Eligard; an M2 protein channel peptide inhibitor; metreleptin; a melanocortin receptor agonist peptide such as bremalanotide/PT-141; a melanocortin; a muramyl tripeptide such as Mepact (mifamurtide); a myelin basic protein peptide such as MBP 8298 (dirucotide); an N-type voltage-gated calcium channel blocker such as Ziconotide (Prialt); a parathyroid hormone peptide; a parathyroid analog such as 768974; a peptide hormone analog such as UGP281; a prostaglandin F2-α receptor inhibitor such as PDC31; a protease inhibitor such as PPL-100; surfaxin; a thromobspondin-1 (TSP-1) mimetic such as CVX-045 or ABT 510; a vasoactive intestinal peptide; vasopressin; a Y2R agonist peptide such as RG7089; obinepeptide; and TM30339.

Drugs as a Payload Conjugated to an Antibody

The payload conjugated to an antibody of the present disclosure may be any of a number of drugs. Exemplary drugs include small molecule drugs and peptide drugs. Thus, the present disclosure provides drug-antibody conjugates, where a drug is covalently linked to fGly of a converted tag of an Ig light chain polypeptide of an antibody.

“Small molecule drug” as used herein refers to a compound, e.g., an organic compound, which exhibits a pharmaceutical activity of interest and which is generally of a molecular weight of no greater than about 800 Da, or no greater than 2000 Da, but can encompass molecules of up to 5 kDa and can be as large as about 10 kDa. A small inorganic molecule refers to a molecule containing no carbon atoms, while a small organic molecule refers to a compound containing at least one carbon atom.

“Peptide drug” as used herein refers to amino-acid containing polymeric compounds, and is meant to encompass naturally-occurring and non-naturally-occurring peptides, oligopeptides, cyclic peptides, polypeptides, and proteins, as well as peptide mimetics. The peptide drugs may be obtained by chemical synthesis or be produced from a genetically encoded source (e.g., recombinant source). Peptide drugs can range in molecular weight, and can be from 200 Da to 10 kDa or greater in molecular weight.

In some cases, the drug is a cancer chemotherapeutic agent. For example, where an antibody has specificity for a tumor cell, the antibody can be modified as described herein to include an aldehyde tag, can be subsequently converted to an fGly-modified antibody, and can then be conjugated to a cancer chemotherapeutic agent. Cancer chemotherapeutic agents include non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones. Peptidic compounds can also be used.

Suitable cancer chemotherapeutic agents include dolastatin and active analogs and derivatives thereof; and auristatin and active analogs and derivatives thereof. See, e.g., WO 96/33212, WO 96/14856, and U.S. Pat. No. 6,323,315. For example, dolastatin 10 or auristatin PE can be included in an antibody-drug conjugate of the present disclosure. Suitable cancer chemotherapeutic agents also include maytansinoids and active analogs and derivatives thereof (see, e.g., EP 1391213; and Liu et al (1996) Proc. Natl. Acad. Sci. USA 93:8618-8623); and duocarmycins and active analogs and derivatives thereof (e.g., including the synthetic analogues, KW-2189 and CB 1-TM1). In some cases, the cancer chemotherapeutic agent includes a pyrrolobenzodiazepine compound.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation; therefore compounds that bind to the estrogen receptor are used to block this activity. Corticosteroids may inhibit T cell proliferation.

Other suitable chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

Taxanes are suitable for use. “Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

Biological response modifiers suitable for use include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) IFN-α; (7) IFN-γ; (8) colony-stimulating factors; and (9) inhibitors of angiogenesis.

Formulations

The antibody conjugates of the present disclosure can be formulated in a variety of different ways. In general, where the antibody conjugate is an antibody-drug conjugate, the antibody conjugate is formulated in a manner compatible with the drug conjugated to the Ig polypeptide (e.g., Ig light chain polypeptide), the condition to be treated, and the route of administration to be used.

The antibody conjugate (e.g., antibody-drug conjugate) can be provided in any suitable form, e.g., in the form of a pharmaceutically acceptable salt, and can be formulated for any suitable route of administration, e.g., oral, topical or parenteral administration. Where the antibody conjugate is provided as a liquid injectable (such as in those embodiments where they are administered intravenously or directly into a tissue), the antibody conjugate can be provided as a ready-to-use dosage form, or as a reconstitutable storage-stable powder or liquid composed of pharmaceutically acceptable carriers and excipients.

Methods for formulating antibody conjugates can be adapted from those available in the art. For example, antibody conjugates can be provided in a pharmaceutical composition comprising an effective amount of an antibody conjugate and a pharmaceutically acceptable carrier (e.g., saline). The pharmaceutical composition may optionally include other additives (e.g., buffers, stabilizers, preservatives, and the like). Of particular interest in some embodiments are formulations that are suitable for administration to a mammal, particularly those that are suitable for administration to a human.

Nucleic Acids, Expression Vectors and Host Cells

The present disclosure provides a nucleic acid encoding Ig light chain polypeptides containing a tag, as well as constructs and host cells containing the nucleic acid. Such nucleic acids comprise a sequence of DNA having an open reading frame that encodes a tagged Ig light chain polypeptide and, in most embodiments, is capable, under appropriate conditions, of being expressed. “Nucleic acid” encompasses DNA, cDNA, mRNA, and vectors comprising such nucleic acids.

The present disclosure provides a recombinant nucleic acid comprising a nucleotide sequence encoding a tagged Ig light chain polypeptide, as described above. The recombinant nucleic acid can include:
1) a nucleotide sequence encoding a tagged Ig light chain constant region (and not an Ig light chain variable region, i.e., where the recombinant nucleic acid lacks a nucleotide sequence encoding an Ig V_{L} domain);
2) a nucleotide sequence encoding a tagged Ig polypeptide, where the Ig polypeptide comprises an Ig V_{L} domain and a tagged Ig light chain constant region;
3) a nucleotide sequence encoding an Ig heavy chain constant region (and not an Ig heavy chain variable region, i.e., where the recombinant nucleic acid lacks a nucleotide sequence encoding an Ig V_{H} domain); and a nucleotide sequence encoding a tagged Ig light chain constant region (and not an Ig light chain variable region, i.e., where the recombinant nucleic acid lacks a nucleotide sequence encoding an Ig V_{L} domain);
4) a nucleotide sequence encoding a tagged Ig heavy chain constant region, as described above, (and not an Ig heavy chain variable region, i.e., where the recombinant nucleic acid lacks a nucleotide sequence encoding an Ig V_{H} domain); and a nucleotide sequence encoding a tagged Ig light chain constant region (and not an Ig light chain variable region, i.e., where the recombinant nucleic acid lacks a nucleotide sequence encoding an Ig V_{L} domain);
5) a nucleotide sequence encoding a first tagged Ig polypeptide, where the first aldehyde-tagged Ig polypeptide comprises an Ig V_{H} domain and a tagged Ig heavy chain constant region; and a nucleotide sequence encoding a second aldehyde-tagged Ig polypeptide, where the second tagged Ig polypeptide comprises an Ig V_{L} domain and a tagged Ig light chain constant region;
6) a nucleotide sequence encoding a first Ig polypeptide, where the first Ig polypeptide comprises an Ig V_{H} domain and an Ig heavy chain constant region; and a nucleotide sequence encoding a second Ig polypeptide, where the second Ig polypeptide includes a tag, where the second Ig polypeptide comprising an Ig V_{L} domain and a tagged Ig light chain constant region.

The present disclosure provides a recombinant expression vector comprising a nucleic acid as described above, where the nucleotide sequence encoding the Ig polypeptide(s) is operably linked to a promoter. In some embodiments, where a subject recombinant expression vector encodes both Ig heavy and light chains (with or without Ig variable regions), the heavy and light chain-encoding sequences can be operably linked to the same promoter, or to separate promoters.

Where a recombinant expression vector includes a nucleotide sequence encoding a heavy chain variable (V_{H}) region and/or a light chain variable (V_{L}) region, it will be appreciated that a large number of V_{H} and V_{L} amino acid sequences, and nucleotide sequences encoding same, are known in the art, and can be used. See, e.g., Kabat et al., Sequences of Proteins of Immunological Interest, 5th Ed. Public Health Service, National Institutes of Health, Bethesda, Md. (1991).

In those instances in which a recombinant expression vector comprises a nucleotide sequence encoding an Ig heavy or Ig light chain without variable region sequences, the vector can include an insertion site for an Ig variable region 5′ of the Ig polypeptide-encoding nucleotide sequence. For example, a recombinant expression vector can comprise, in order from 5′ to 3′:
1) an insertion site for a nucleotide sequence encoding a V_{L} domain; and a nucleotide sequence encoding a tagged Ig light chain constant region; or
2) an insertion site for a nucleotide sequence encoding a V_{H} domain; and a nucleotide sequence encoding an Ig heavy chain constant region, which may or may not include a tag.

Nucleic acids contemplated herein can be provided as part of a vector (also referred to as a construct), a wide variety of which are known in the art. Exemplary vectors include, but are not limited to, plasmids; cosmids; viral vectors (e.g., retroviral vectors); non-viral vectors; artificial chromosomes (yeast artificial chromosomes (YAC's), BAC's, etc.); mini-chromosomes; and the like. The choice of vector will depend upon a variety of factors such as the type of cell in which propagation is desired and the purpose of propagation.

Vectors can provide for extrachromosomal maintenance in a host cell or can provide for integration into the host cell genome. Vectors are amply described in numerous publications well known to those in the art, including, e.g., Short Protocols in Molecular Biology, (1999) F. Ausubel, et al., eds., Wiley & Sons. Vectors may provide for expression of the nucleic acids encoding a polypeptide of interest (e.g., a tagged polypeptide, an FGE, etc.), may provide for propagating the subject nucleic acids, or both.

Exemplary vectors that may be used include but are not limited to those derived from recombinant bacteriophage DNA, plasmid DNA or cosmid DNA. For example, plasmid vectors such as pBR322, pUC 19/18, pUC 118, 119 and the M13 mp series of vectors may be used. Bacteriophage vectors may include λgt10, λgt11, λgt18-23, λZAP/R and the EMBL series of bacteriophage vectors. Cosmid vectors that may be utilized include, but are not limited to, pJB8, pCV 103, pCV 107, pCV 108, pTM, pMCS, pNNL, pHSG274, COS202, COS203, pWE15, pWE16 and the charomid 9 series of vectors. Alternatively, recombinant virus vectors may be engineered, including but not limited to those derived from viruses such as herpes virus, retroviruses, vaccinia virus, poxviruses, adenoviruses, adeno-associated viruses, or bovine papilloma virus.

For expression of a protein of interest (e.g., a tagged Ig polypeptide or an FGE), an expression cassette may be employed. Thus, the present invention provides a recombinant expression vector comprising a subject nucleic acid. The expression vector provides a transcriptional and translational regulatory sequence, and may provide for inducible or constitutive expression, where the coding region is operably linked under the transcriptional control of the transcriptional initiation region, and a transcriptional and translational termination region. These control regions may be native to the gene encoding the polypeptide (e.g., the Ig polypeptide or the FGE), or may be derived from exogenous sources. In general, the transcriptional and translational regulatory sequences may include, but are not limited to, promoter sequences, ribosomal binding sites, transcriptional start and stop sequences, translational start and stop sequences, and enhancer or activator sequences. In addition to constitutive and inducible promoters, strong promoters (e.g., T7, CMV, and the like) find use in the constructs described herein, particularly where high expression levels are desired in an in vivo (cell-based) or in an in vitro expression system. Further exemplary promoters include mouse mammary tumor virus (MMTV) promoters, Rous sarcoma virus (RSV) promoters, adenovirus promoters, the promoter from the immediate early gene of human CMV (Boshart et al., Cell 41:521-530, 1985), and the promoter from the long terminal repeat (LTR) of RSV (Gorman et al., Proc. Natl. Acad. Sci. USA 79:6777-6781, 1982). The promoter can also be provided by, for example, a 5′UTR of a retrovirus.

Expression vectors generally have convenient restriction sites located near the promoter sequence to provide for the insertion of nucleic acid sequences encoding proteins of interest. A selectable marker operative in the expression host may be present to facilitate selection of cells containing the vector. In addition, the expression construct may include additional elements. For example, the expression vector may have one or two replication systems, thus allowing it to be maintained in organisms, for example in mammalian or insect cells for expression and in a prokaryotic host for cloning and amplification. In addition the expression construct may contain a selectable marker gene to allow the selection of transformed host cells. Selection genes are well known in the art and will vary with the host cell used.

Expression constructs encoding tagged Ig polypeptides can also be generated using amplification methods (e.g., a polymerase chain reaction (PCR)), where at least one amplification primer (i.e., at least one of a forward or reverse primer) includes a nucleic acid sequence encoding an aldehyde tag. For example, an amplification primer having a tag amino acid sequence-encoding nucleotide sequence is designed to provide for amplification of a nucleic acid encoding an Ig polypeptide. The extension product that results from polymerase-mediated synthesis from the tagged forward primer produces a nucleic acid amplification product encoding a fusion protein composed of a tagged Ig polypeptide. The amplification product is then inserted into an expression construct of choice to provide a tagged polypeptide expression construct.

Host Cells

The present disclosure provides genetically modified host cells comprising a subject nucleic acid, including a genetically modified host cell comprising a recombinant expression vector as described above. Any of a number of suitable host cells can be used in the production of an antibody containing the present tagged Ig light chain polypeptide. The host cell used for production of an antibody containing the tagged Ig polypeptide can optionally provide for FGE-mediated conversion, so that the antibody produced contains an fGly-modified Ig polypeptide, where the tag is converted to contain fGly, following expression and modification by FGE. Alternatively the host cell can provide for production of an antibody containing a tagged and unconverted Ig light chain polypeptide (e.g., due to lack of expression of an FGE that facilitates conversion of the tag).

The aldehyde moiety of a converted tag can be used for a variety of applications including, but not limited to, visualization using fluorescence or epitope labeling (e.g., electron microscopy using gold particles equipped with aldehyde reactive groups); protein immobilization (e.g., protein microarray production); protein dynamics and localization studies and applications; and conjugation of proteins with a payload (e.g., moieties that improve a parent protein's half-life (e.g., poly(ethylene glycol)), targeting moieties (e.g., to enhance delivery to a site of action), and biologically active moieties (e.g., a therapeutic moiety).

In general, the polypeptides described herein may be expressed in prokaryotes or eukaryotes in accordance with conventional ways, depending upon the purpose for expression. Thus, the present invention further provides a host cell, e.g., a genetically modified host cell that comprises a nucleic acid encoding a tagged polypeptide. The host cell can further optionally comprise a recombinant FGE, which may be endogenous or heterologous to the host cell. Thus, in some cases, the host cell is genetically modified to express an FGE.

Host cells for production (including large scale production) of a tagged and unconverted, or (where the host cell expresses a suitable FGE) tagged and converted Ig polypeptide, or for production of an FGE (e.g., for use in a cell-free method) can be selected from any of a variety of available host cells. Exemplary host cells include those of a prokaryotic or eukaryotic unicellular organism, such as bacteria (e.g., Escherichia coli strains, Bacillus spp. (e.g., B. subtilis), and the like) yeast or fungi (e.g., S. cerevisiae, Pichia spp., and the like), and other such host cells can be used. Exemplary host cells originally derived from a higher organism such as insects, vertebrates, particularly mammals, (e.g. CHO, HEK, and the like), may be used as the expression host cells.

Suitable mammalian cell lines include, but are not limited to, HeLa cells (e.g., American Type Culture Collection (ATCC) No. CCL-2), CHO cells (e.g., ATCC Nos. CRL9618 and CRL9096), CHO DG44 cells (Urlaub (1983) Cell 33:405), CHO-K1 cells (ATCC CCL-61), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

Specific expression systems of interest include bacterial, yeast, insect cell and mammalian cell derived expression systems. Representative systems from each of these categories are provided below.

The product can be recovered by any appropriate means known in the art. Further, any convenient protein purification procedures may be employed, where suitable protein purification methodologies are described in Guide to Protein Purification, (Deuthser ed.) (Academic Press, 1990). For example, a lysate may prepared from a cell comprising the expression vector expressing the tagged Ig polypeptide, and purified using high performance liquid chromatography (HPLC), exclusion chromatography, gel electrophoresis, affinity chromatography, and the like.

Methods

Methods for Conversion and Modification of a Tag

Conversion of a tag, e.g., a sulfatase motif in a tag, present in a tagged Ig polypeptide of an antibody can be accomplished by cell-based (in vivo) or cell-free methods (in vitro). Similarly, modification of a converted tag of a tagged polypeptide can be accomplished by cell-based (in vivo) or cell-free methods (in vitro). These are described in more detail below.

“In Vivo” Host Cells Conversion and Modification

Conversion of a tag, e.g., a sulfatase motif in a tag, of an aldehyde tagged polypeptide of an antibody can be accomplished by expression of the tagged polypeptide in a cell that contains a suitable FGE. In this embodiment, conversion of the cysteine or serine of the tag occurs during or following translation in the host cell. The FGE of the host cell can be endogenous to the host cell, or the host cell can be recombinant for a suitable FGE that is heterologous to the host cell. FGE expression can be provided by an expression system endogenous to the FGE gene (e.g., expression is provided by a promoter and other control elements present in the native FGE gene of the host cell), or can be provided by from a recombinant expression system in which the FGE coding sequence is operably linked to a heterologous promoter to provide for constitutive or inducible expression.

Conditions suitable for use to accomplish conjugation of a reactive partner moiety to a tagged polypeptide are similar to those described in Mahal et al. (1997 May 16) Science 276(5315):1125-8.

In some instances, where the present method is carried out in a cell, the cell is in vitro, e.g., in in vitro cell culture, e.g., where the cell is cultured in vitro in a single-cell suspension or as an adherent cell. In some embodiments, the cell is cultured in the presence of an oxidation reagent that can activate FGE. In some embodiments, a cell expressing an FGE is cultured in the presence of a suitable amount of Cu²⁺ in the culture medium. In certain aspects, the Cu²⁺ is present in the cell culture medium at a concentration of from 1 nM to 100 mM, such as from 0.1 μM to 10 mM, from 1 μM to 1 mM, from 2 μM to 500 μM, from 4 μM to 300 μM, or from 5 μM to 200 μM (e.g., from 10 μM to 150 μM). The culture medium may be supplemented with any suitable copper salt to provide for the Cu²⁺. Suitable copper salts include, but are not limited to, copper sulfate (i.e., copper(II) sulfate, CuSO₄), copper citrate, copper tartrate, copper nitrate, and any combination thereof.

“In Vitro” (Cell-Free) Conversion and Modification

In vitro (cell-free) conversion of a tag, e.g., a sulfatase motif in a tag, of a tagged Ig polypeptide of an antibody can be accomplished by contacting a tagged polypeptide with an FGE under conditions suitable for conversion of a cysteine or serine of a sulfatase motif of the tag to an fGly. For example, nucleic acid encoding a tagged Ig polypeptide can be expressed in an in vitro transcription/translation system in the presence of a suitable FGE to provide for production of tagged and converted Ig polypeptides.

Alternatively, isolated, unconverted, tagged Ig polypeptide can be isolated following recombinant production in a host cell lacking a suitable FGE or by synthetic production. The isolated tagged Ig polypeptide is then contacted with a suitable FGE under conditions to provide for tag conversion. The tagged Ig polypeptide can be unfolded by methods known in the art (e.g., using heat, adjustment of pH, chaotropic agents, (e.g., urea, and the like), organic solvents (e.g., hydrocarbons: octane, benzene, chloroform), etc.) and the denatured protein contacted with a suitable FGE. The tagged Ig polypeptide can then be refolded under suitable conditions.

With respect to modification of tagged and converted Ig polypeptide of an antibody, e.g., to covalently and site-specifically attach a payload (e.g., drug) thereto, modification is normally carried out in vitro. An antibody containing a converted aldehyde tagged Ig polypeptide is isolated from a production source (e.g., recombinant host cell production, synthetic production), and contacted with a reactive partner-containing drug or other moiety under conditions suitable to provide for conjugation of the drug or other moiety to the fGly of the tag in the Ig polypeptide, e.g., Ig light chain polypeptide, of the antibody.

In some instances, a combination of cell-based conversion and cell-free conversion is carried out, to generate a converted tag; followed by cell-free modification of the converted tag. In some embodiments, a combination of cell-free conversion and cell-based conversion is carried out.

Method of Producing an Antibody Conjugate

Aspects of the present disclosure include a method of producing an antibody conjugate, as described herein. In general terms, the method may include combining, in a reaction mixture, an fGly-modified antibody having a converted tag in its Ig light chain polypeptide, as described above, and a reactive partner, e.g., an aldehyde-reactive reactive partner, that includes the payload (e.g., drug) and an aldehyde-reactive group. In some cases, the reactive partner may be represented by the formula: P-(L)-R, where P is the payload covalently linked to R, an aldehyde-reactive group, through an optional linking group L. Under suitable conditions, the aldehyde-reactive group may react with the aldehyde group of the fGly in the converted tag of the fGly-modified antibody (“A”) in the reaction mixture, to form a covalent linkage between the payload (e.g., drug) and the fGly-modified antibody at the fGly residue of the converted tag (which may be represented by the formula: P-(L)-A, or P-(L)-A-(L)-P, etc., depending on the number of tags present in each of the Ig polypeptides of the antibody). The reaction may be carried out in any suitable condition, such as those described in, e.g., US20120183566, US20140141025 and WO2014074218, each of which is incorporated herein by reference.

The payload (P) may be any suitable moiety (e.g., drug, water-soluble polymer, detectable label, synthetic peptide, etc.) as described above, and may be a compound that can be functionalized with an aldehyde-reactive group. The aldehyde-reactive group (R) may be any suitable functional group suitable for carrying out a conjugation reaction between the present fGly-modified antibody and the reactive partner. In some cases, the aldehyde-reactive group is an α-nucleophile, such as an aminooxy or hydrazide group. Suitable aldehyde-reactive groups include, without limitation, a hydrazine compound, hydrazide compound, aminooxy compound, semicarbazide (e.g., thiosemicarbazide) compound, hydrazinyl-indole compound, hydrazinyl-imidazole compound, hydrazinyl-pyrrole compound, hydrazinyl-furan compound, and a pyrazolinone compound.

In some embodiments, the reactive partner includes a payload (P) (e.g., drug) attached to an aldehyde-reactive group (R) that is based on a hydrazinyl-indole group, and can be produced using any suitable method, e.g., as described in US20140141025, which is incorporated herein by reference. A hydrazinyl-indole-containing reactive partner may react with an aldehyde of fGly in a converted tag in an fGly-modified antibody, as described herein, where the hydrazine of the hydrazinyl-indole coupling moiety undergoes an intramolecular cyclization to form a partially unsaturated pyrazole or pyridazine ring, to covalently attach the payload (e.g., drug) to the antibody Ig light chain polypeptide. Alternatively, the hydrazine of the hydrazinyl-indole coupling moiety may undergo an intramolecular cyclization to form a partially unsaturated pyridazine or 1,2-diazepine ring, to covalently attach the payload (e.g., drug) to the antibody Ig light chain polypeptide.

In some cases, the reactive partner includes a payload (P) (e.g., drug) attached to an aldehyde-reactive group (R) based on a pyrazolinone group, and can be produced using any suitable method, e.g., as described in WO2014074218, which is incorporated herein by reference. A pyrazolinone-containing reactive partner may react with an aldehyde of fGly in a converted tag in an fGly-modified antibody, as described herein, to covalently attach the payload (e.g., drug) of the reactive partner to the antibody Ig light chain polypeptide through a cyclic linkage.

In some cases, the reactive partner includes a payload (P) (e.g., drug) attached to an aldehyde-reactive group (R) based on a hydrazinyl-substituted heteroaryl ring compound, such as a hydrazinyl-substituted 5-membered heteroaryl ring compound, where one or more atoms in the ring is a heteroatom (e.g., N, O or S). The hydrazinyl-substituted heteroaryl ring compound may include a hydrazinyl-imidazole compound, hydrazinyl-pyrrole compound, or a hydrazinyl-furan compound. Thus, a hydrazinyl-substituted heteroaryl ring compound (e.g., a hydrazinyl-imidazole compound, hydrazinyl-pyrrole compound, a hydrazinyl-furan compound) may react with an aldehyde of fGly in a converted tag in an fGly-modified antibody, as described herein, to covalently attach the payload (e.g., drug) to the antibody Ig light chain polypeptide through a cyclic linkage.

The reactive partner may further include a linking group (L) bridging the payload (P) (e.g., drug) and the aldehyde-reactive group (R) through covalent bonds. The linking group may be any suitable linking group. In some cases, the linking group includes polyethylene glycol (PEG); amino acids; alkyl groups, including substituted alkyl groups; a protease cleavable group; esters; acyloxy groups, including substituted acyloxy groups, etc. Suitable linking groups and methods of using the same to bridge a payload (e.g., drug) and an aldehyde-reactive group are described in, e.g., US20150157736, which is incorporated by reference herein. In some embodiments, the linking group includes a 4-aminopiperidine (4AP) derivative.

In some cases, the payload is a drug, e.g., a peptide drug. In some cases, peptide drugs to be conjugated to a tagged and converted Ig polypeptide of an fGly-modified antibody can be modified to incorporate an aldehyde-reactive group for reaction with an aldehyde of the fGly residue of the tagged and converted Ig polypeptide. Since the methods of tagged and converted polypeptide modification are compatible with conventional chemical processes, any of a wide variety of commercially available reagents can be used to accomplish conjugation. For example, aminooxy, hydrazide, hydrazine, thiosemicarbazide, hydrazinyl-indole, hydrazinyl-imidazole, hydrazinyl-pyrrole, hydrazinyl-furan or pyrazolinone derivatives of a number of moieties of interest are suitable reactive partners, and are readily available or can be generated using standard chemical methods.

Where the drug is a peptide drug, the reactive moiety (e.g., aminooxy or hydrazide can be positioned at an N-terminal region, the N-terminus, a C-terminal region, the C-terminus, or at a position internal to the peptide. For example, one method involves synthesizing a peptide drug having an aminooxy group. In this example, the peptide is synthesized from a Boc-protected precursor. An amino group of a peptide can react with a compound comprising a carboxylic acid group and oxy-N-Boc group. As an example, the amino group of the peptide reacts with 3-(2,5-dioxopyrrolidin-1-yloxy)propanoic acid. Other variations on the compound comprising a carboxylic acid group and oxy-N-protecting group can include different number of carbons in the alkylene linker and substituents on the alkylene linker. The reaction between the amino group of the peptide and the compound comprising a carboxylic acid group and oxy-N-protecting group occurs through standard peptide coupling chemistry. Examples of peptide coupling reagents that can be used include, but not limited to, DCC (dicyclohexylcarbodiimide), DIC (diisopropylcarbodiimide), di-p-toluoylcarbodiimide, BDP (1-benzotriazole diethylphosphate-1-cyclohexyl-3-(2-morpholinylethyl)carbodiimide), EDC (1-(3-dimethylaminopropyl-3-ethyl-carbodiimide hydrochloride), cyanuric fluoride, cyanuric chloride, TFFH (tetramethyl fluoroformamidinium hexafluorophosphosphate), DPPA (diphenylphosphorazidate), BOP (benzotriazol-1-yloxytris(dimethylamino)phosphonium hexafluorophosphate), HBTU (O-benzotriazol-1-yl-N,N,N′,N′-tetramethyluronium hexafluorophosphate), TBTU (O-benzotriazol-1-yl-N,N,N′,N′-tetramethyluronium tetrafluoroborate), TSTU (O—(N-succinimidyl)-N,N,N′,N′-tetramethyluronium tetrafluoroborate), HATU (N-[(dimethylamino)-1-H-1,2,3-triazolo[4,5,6]-pyridin-1-ylmethylene]- -N-methylmethanaminium hexafluorophosphate N-oxide), BOP-Cl (bis(2-oxo-3-oxazolidinyl)phosphinic chloride), PyBOP ((1-H-1,2,3-benzotriazol-1-yloxy)-tris(pyrrolidino)phosphonium tetrafluorophopsphate), BrOP (bromotris(dimethylamino)phosphonium hexafluorophosphate), DEPBT (3-(diethoxyphosphoryloxy)-1,2,3-benzotriazin-4(3H)-one) PyBrOP (bromotris(pyrrolidino)phosphonium hexafluorophosphate). As a non-limiting example, HOBt and DIC can be used as peptide coupling reagents.

Deprotection to expose the amino-oxy functionality is performed on the peptide comprising an N-protecting group. Deprotection of the N-oxysuccinimide group, for example, occurs according to standard deprotection conditions for a cyclic amide group. Deprotecting conditions can be found in Greene and Wuts, Protective Groups in Organic Chemistry, 3rd Ed., 1999, John Wiley & Sons, NY and Harrison et al. Certain deprotection conditions include a hydrazine reagent, amino reagent, or sodium borohydride. Deprotection of a Boc protecting group can occur with TFA. Other reagents for deprotection include, but are not limited to, hydrazine, methylhydrazine, phenylhydrazine, sodium borohydride, and methylamine. The product and intermediates can be purified by conventional means, such as HPLC purification.

The ordinarily skilled artisan will appreciate that factors such as pH and steric hindrance (i.e., the accessibility of the aldehyde tag to reaction with a reactive partner of interest) are of importance. Modifying reaction conditions to provide for optimal conjugation conditions is well within the skill of the ordinary artisan, and is routine in the art. In general, it is normally desirable to conduct conjugation reactions at a pH below 7, with a pH of about 5.5, about 6, about 6.5, usually about 5.5 being optimal. Where conjugation is conducted with a tagged and converted polypeptide present in or on a living cell, the conditions are selected so as to be physiologically compatible. For example, the pH can be dropped temporarily for a time sufficient to allow for the reaction to occur but within a period tolerated by the cell having an aldehyde tag (e.g., from about 30 min to 1 hour). Physiological conditions for conducting modification of tagged and converted polypeptides on a cell surface can be similar to those used in a ketone-azide reaction in modification of cells bearing cell-surface azides (see, e.g., U.S. Pat. No. 6,570,040).

Small molecule compounds containing, or modified to contain, an α-nucleophilic group that serves as a reactive partner with an aldehyde of an fGly of a converted tag are also contemplated for use as drugs in the Ig-drug conjugates of the present disclosure. General methods are known in the art for chemical synthetic schemes and conditions useful for synthesizing a compound of interest (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Thus small molecules having an aminooxy or hydrazone group for reaction with an aldehyde of an fGly of a tagged and converted Ig polypeptide are available or can be readily synthesized. An aminooxy or hydrazone group can be installed onto a small molecule using standard synthetic chemistry techniques.

Method of Treating an Individual

The antibody-drug conjugates of the present disclosure find use in treatment of a condition or disease in a subject that is amenable to treatment by administration of the parent drug (i.e., the drug prior to conjugation to the antibody). By “treatment” is meant that at least an amelioration of the symptoms associated with the condition afflicting the host is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g., prevented from happening, or stopped, e.g. terminated, such that the host no longer suffers from the condition, or at least the symptoms that characterize the condition. Thus treatment includes: (i) prevention, that is, reducing the risk of development of clinical symptoms, including causing the clinical symptoms not to develop, e.g., preventing disease progression to a harmful state; (ii) inhibition, that is, arresting the development or further development of clinical symptoms, e.g., mitigating or completely inhibiting an active disease; and/or (iii) relief, that is, causing the regression of clinical symptoms.

In the context of cancer, the term “treating” includes any or all of: reducing growth of a solid tumor, inhibiting replication of cancer cells, reducing overall tumor burden, and ameliorating one or more symptoms associated with a cancer. Thus, the present disclosure provides methods for delivering a cancer chemotherapeutic agent to an individual having a cancer. The methods are useful for treating a wide variety of cancers, including carcinomas, sarcomas, leukemias, and lymphomas. The cancer treated by the present method may be a cancer of a variety of tissues organs, such as, without limitation, cancer of the lungs, liver, breast, prostate, ovary, kidney, brain, colon, intestine, spleen, stomach, mouth, throat, skin, blood cells, etc.

The antibody to which the payload, e.g., drug, such as a cancer chemotherapeutic agent, is bound may specifically bind to an antigen associated with cell(s) or tissue(s) that are to be targeted and acted upon by the payload.

The present method may include administering to an individual a therapeutically effective amount of an antibody conjugate, e.g., an antibody-drug conjugate, as described herein. The antibody conjugate may be in any suitable formulation, e.g., formulated with a pharmaceutically acceptable excipient, as described herein.

The subject to be treated can be one that is in need of therapy, where the host to be treated is one amenable to treatment using the parent drug. Accordingly, a variety of subjects may be amenable to treatment using an antibody-drug conjugates disclosed herein. Generally such subjects are “mammals”, with humans being of particular interest. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys.

The amount of antibody-drug conjugate administered can be initially determined based on guidance of a dose and/or dosage regimen of the parent drug. In general, the antibody-drug conjugates can provide for targeted delivery and/or enhanced serum half-life of the bound drug, thus providing for at least one of reduced dose or reduced administrations in a dosage regimen. Thus the antibody-drug conjugates can provide for reduced dose and/or reduced administration in a dosage regimen relative to the parent drug prior to being conjugated in an Ig-drug conjugate of the present disclosure.

Furthermore, as noted above, because the antibody-drug conjugates can provide for controlled stoichiometry of drug delivery, dosages of antibody-drug conjugates can be calculated based on the number of drug molecules provided on a per antibody-drug conjugate basis.

In some embodiments, multiple doses of an antibody-drug conjugate are administered. The frequency of administration of an antibody-drug conjugate can vary depending on any of a variety of factors, e.g., severity of the symptoms, etc. For example, in some embodiments, an Ig-drug conjugate is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

### EXAMPLES

The following examples are put forth so as to provide those of ordinary skill in the art with a complete disclosure and description of how to make and use the disclosed subject matter, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Production of Tagged Ig Light Chain Constructs and Antibodies Containing Tagged Light Chains

Positions within the constant region of human kappa light chain was systematically scanned by a tag insertion. A collection of tagged light chain constructs were generated by inserting the tag sequence: LCTPSR (SEQ ID NO:158) between adjacent amino acids at different sites in the light chain constant region (FIG. 1). Each construct also contained a light chain variable region of an antibody specific for a cell surface antigen. The entire length of the light chain constant region was scanned, to generate 106 variants, each having the tag inserted at a different position (see, e.g., FIGS. 10A-10D). Each light chain variant was provided in an expression vector for expression in Chinese hamster ovary (CHO) cells.

### Methods and Materials

Light chain expression vector for the antigen-specific antibody was generated and digested with BamHI and DraIII in order to remove wild type human kappa light chain constant region (FIG. 11). The digested plasmid DNA was purified by gel electrophoresis and QIAquick gel extraction kit (Qiagen, MD). The purified plasmid backbone was used for cloning variant human kappa light chain constant genes containing a tag in various positions.

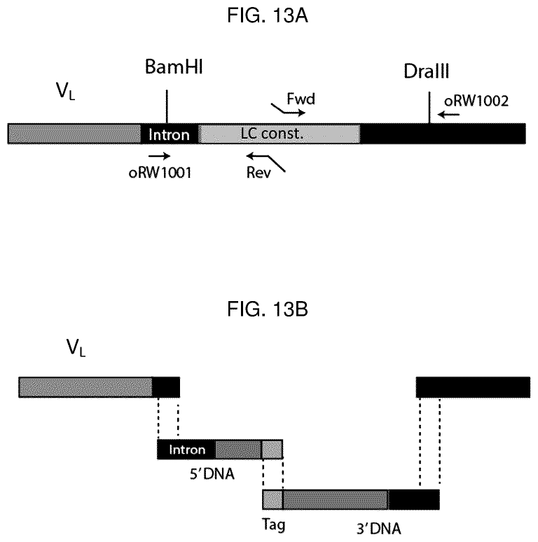

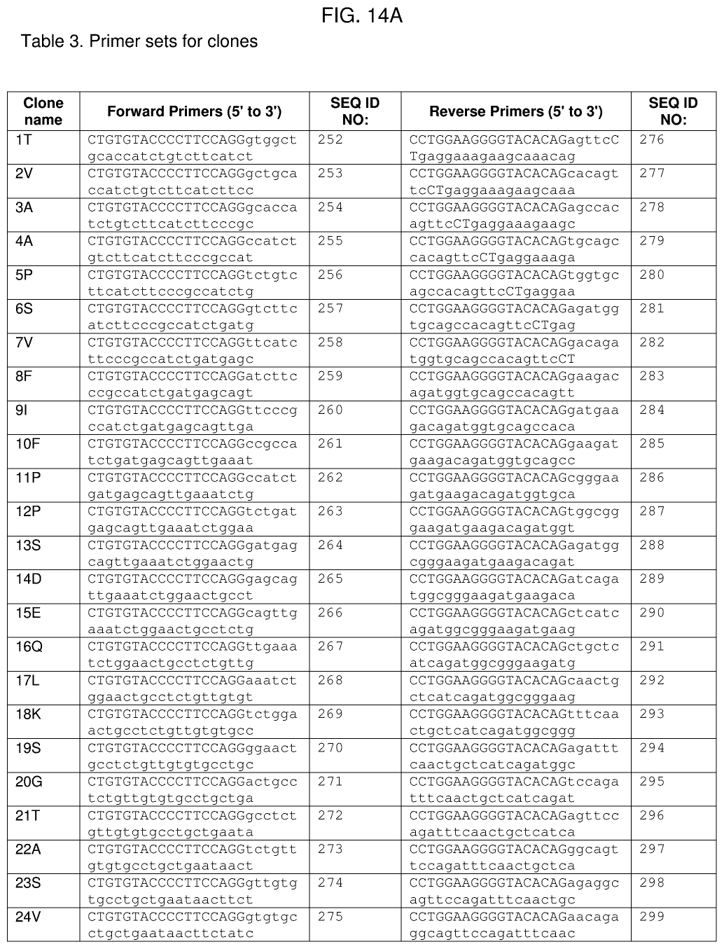

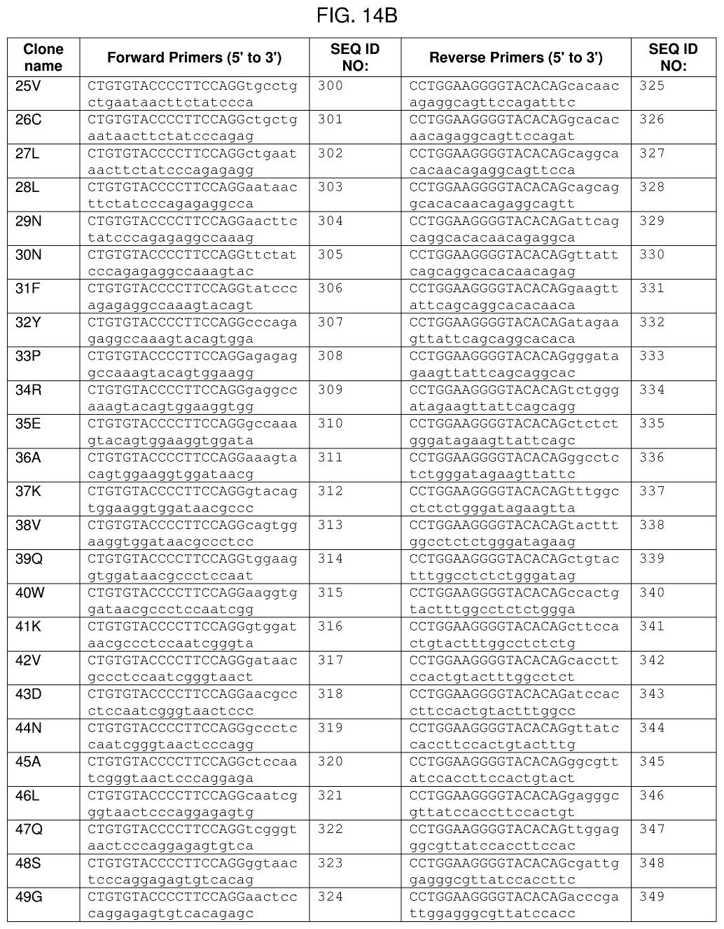

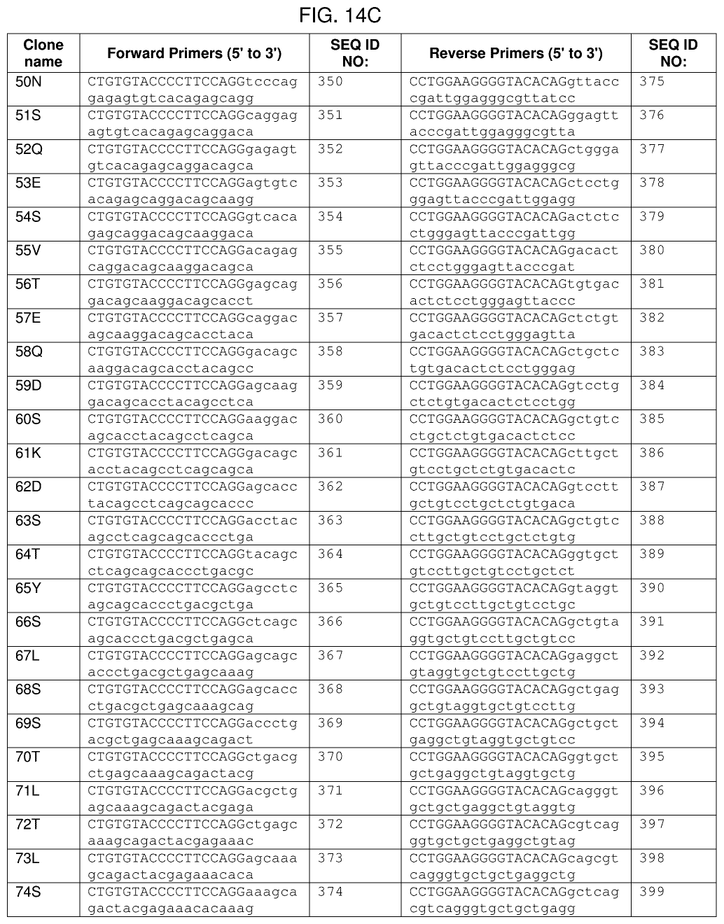

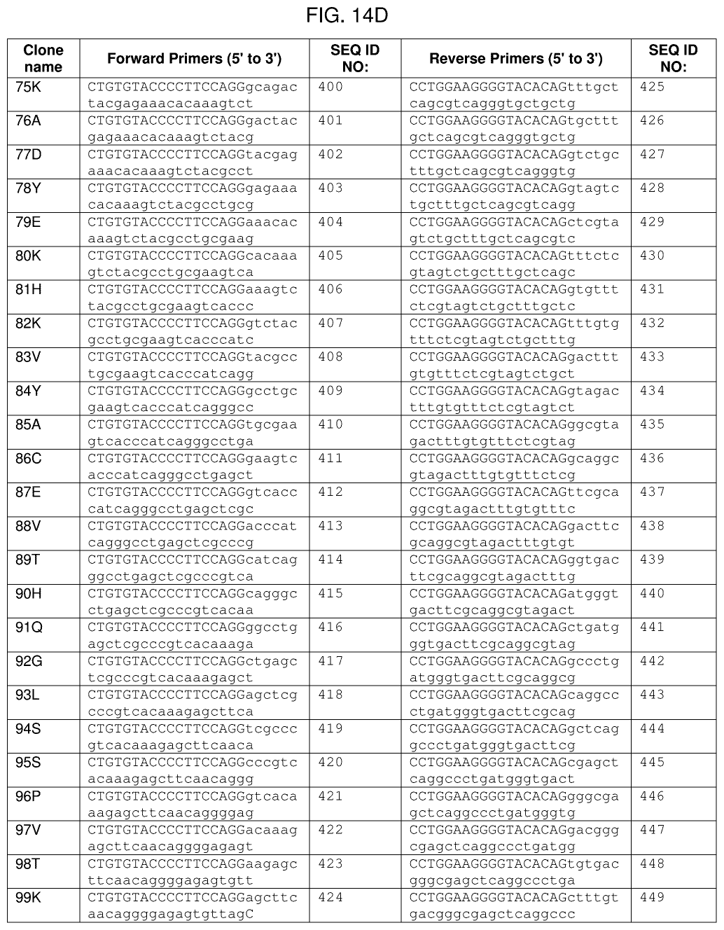

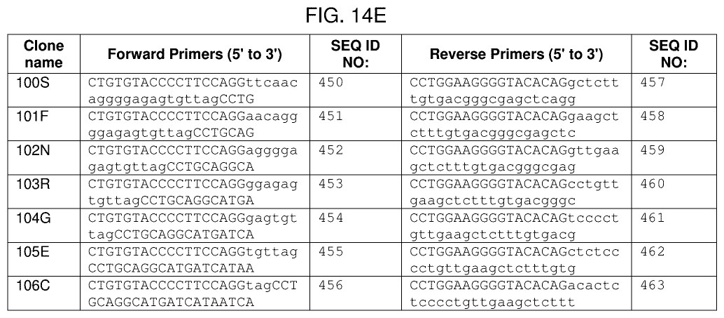

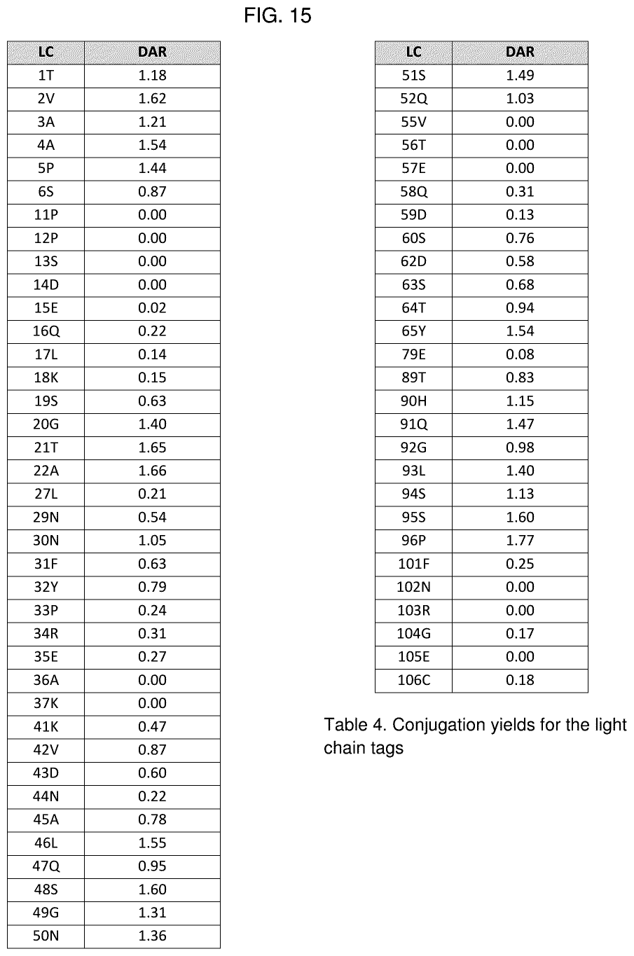

For inserting a tag into human kappa light chain constant region, two PCR amplifications were performed using the light chain expression vector as a template with Phusion DNA polymerase (New England Biolabs, MA). PCR cycling condition included a preheating step at 98° C. for 1 min, followed by 30 cycle of 98° C. for 10 seconds, 60° C. for 10 seconds, 72° C. for 20 seconds followed by 72° C. for 1 min for final extension. PCR amplification for the 5′ part of human kappa light chain constant region was performed using a reverse primer (Table 3, in FIGS. 14A-14E, and FIG. 13A) with oRW1001 (5′ TCAAACGTGAGTAGAATTTAAACTTT 3′ (SEQ ID NO:250)) and the 3′ part of the DNA fragments were amplified using a forward primer and oRW1002 (5′ AAAGGGCGAAAAACCGTCTATCAGG 3′ (SEQ ID NO:251)) (Table 3 and FIG. 13A). All pairs of forward and reverse primers were designed to insert a tag sequence, LCTPSR (SEQ ID NO:158), throughout the human kappa light chain constant region (Table 3 in FIGS. 14A-14E). The amplified 5′ DNA fragment and the corresponding 3′ DNA fragment had compatible overlaps for assembly, as shown in the FIG. 13B. The amplified 5′ and 3′ DNA fragments were combined resulting in total 106 pairs of DNA mixture. The vector was linearized using BamHI and DraIII, and was added to the DNA mixtures. The reaction was subjected to Gibson assembly using Gibson assembly master mix (New England Biolabs, MA) according to the manufacturer's protocol.

The assembled DNA was transformed into E. coli Top10 chemically competent cells (Thermo Fisher Scientific) by the heat shock method. For this purpose 3 μl of the assembled plasmid DNA was added to 50 μl of chemically competent E. coli Top10 and the mixture was incubated on ice for 30 minutes and then subjected to a heat shock at 42° C. for 45 seconds. Then, the suspension was immediately placed on ice for one minute and 500 μl of SOC medium (Teknova, CA) was added. This mixture was incubated for 1 hour at 37° C. on shaker. These cells were plated on LB agar media (Teknova, CA) with antibiotic carbenicillin (100 g/ml) as selection marker and grown overnight at 37° C. incubator. Colonies appeared on the agar plate were individually picked and inoculated into 3 ml of LB broth and grown at 37° C. for overnight followed by plasmid DNA purification using plasmid DNA isolation kit (Qiagen, Germany) according to the manufacturer's protocol. All 106 clones' DNA sequence integrity was confirmed by sending out to a sequencing service vendor (Sequetech, CA).

### Example 2: Analysis of Expression Titer of Antibodies with a Tag in the Light Chain

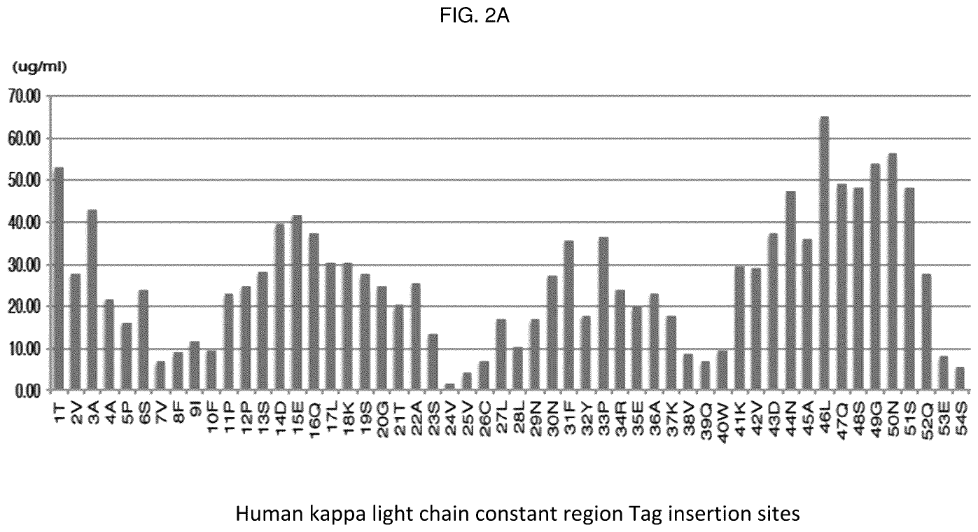

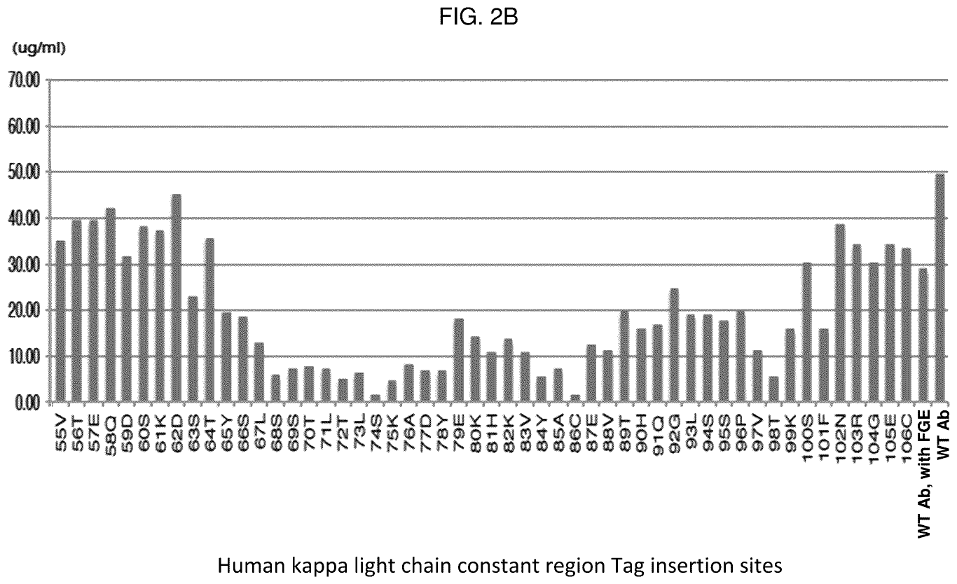

The effect of inserting a tag at different positions along the light chain on the titer of expression of the antibody was tested by transfecting CHO cells (ExpiCHO™ cells) with an expression vector containing each of the variant light chains generated as described in Example 1, together with a second expression vector encoding the heavy chain polypeptide of the antigen-specific antibody, and a third expression vector encoding a formylglycine generating enzyme (FGE). Then the amount of antibodies secreted into the culture medium by cells expressing antibodies having the tag inserted along different positions of the light chain constant region was measured. The measured antibody titer showed that insertion position affected efficiency of expression from the CHO cells (FIGS. 2A and 2B).

FIGS. 2A and 2B: The expression titer (y-axis), in ExpiCHO™ cells, of variant tagged antibodies, each having a sulfatase motif inserted adjacent and C-terminal to the position indicated, as defined relative to SEQ ID NO:1, in the constant region of its Ig light chain amino acid sequence. The titer of expression of control antibodies having no tag insertion in the light chain, from cells where FGE is co-expressed (“WT Ab with FGE”) or not co-expressed (“WT Ab”), is shown in FIG. 2B.

### Materials and Methods

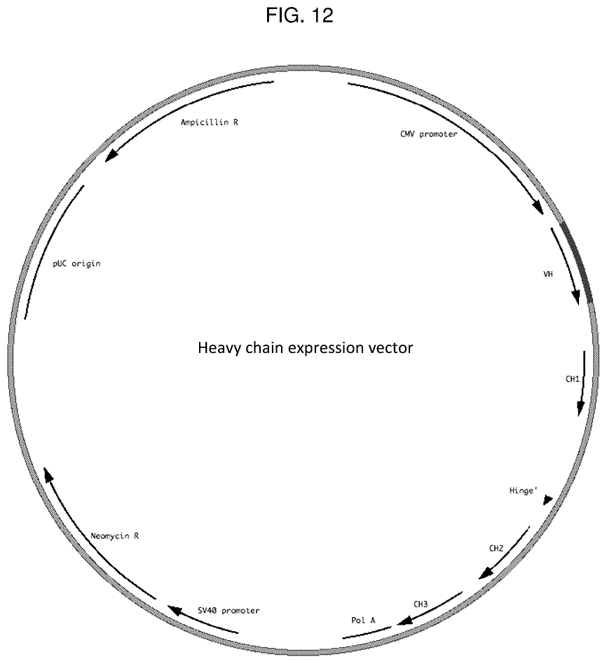

Expi-CHO-S cells were maintained routinely in 150 ml shaking flask in CHO expression medium (Thermo Fisher Scientific) at 37° C., and 8% CO₂. One day before transfection Expi-CHO-S cells were seeded into fresh CHO expression medium with the final cell density of 4×10⁶ cells/ml. On next day, the cell number in the suspension culture was determined by using TC20 cell counter (Bio-Rad, CA) and adjusted the cell density to 6×10⁶ cells/ml by adding additional CHO expression medium. At this step, 100 mM of CuSO₄ was supplemented to a final concentration of 100 μM. 6 ml of cells were seeded in a disposable mini-bioreactor tube (Corning, NY) for transfection. 1.8 ug of the expression vector for the heavy chain (FIG. 12) and 1.5 ug of FGE expression plasmid DNA were mixed with 2.7 ug of the light chain expression vector, as described above, in 240 μl of Opti-SFM (Thermo Fisher Scientific, CA) followed by combining with lipofectamine mixture containing 19.2 ul of Expi-ChoFectamine in 240 ul of Opti-SFM.

The Expi-CHO Fectamine and DNA complex was directly added to the cells and briefly mixed by swirling. After culturing at 37° C., and 8% CO₂ with 180 rpm orbital shaking for a day, 36 μl of enhancer solution and 960 μl of feed provided in the Expi-CHO transfection kit (Thermo Fisher Scientific, CA) was added to the cell and the cells were kept at 37° C. and 8% CO₂ with 180 rpm orbital agitation. After 4 days, additional 960 ul of feed was added to the cell and the cells were kept in the same culture condition for 5 days more. The culture supernatant was harvested by centrifugation and filtration with 0.45 m PES filter followed by IgG quantification with Blitz system (Forte Bio, CA) using protein A biosensor chips (FIGS. 2A and 2B).

### Example 3: Analysis of Aggregation of Antibodies with a Tag in the Light Chain

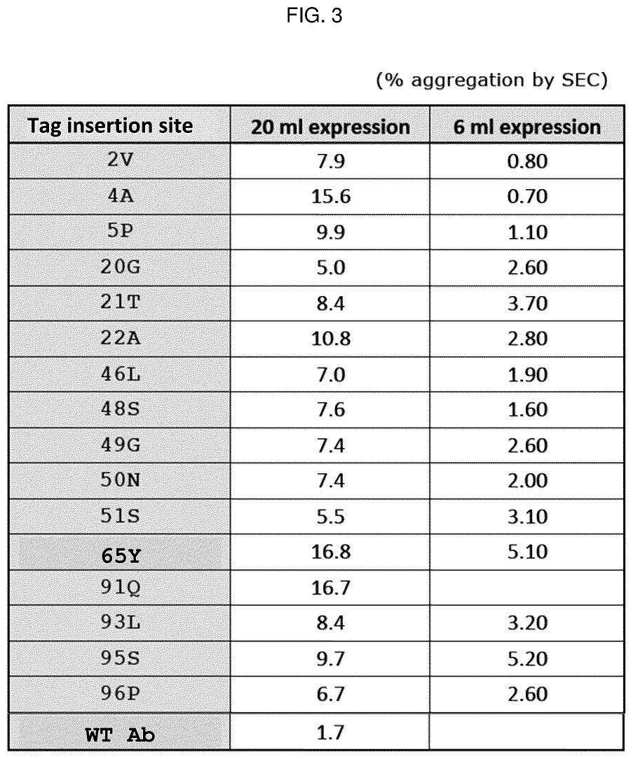

The effect of inserting a tag at different positions along the light chain on aggregation of the tagged antibodies, each having a sulfatase motif inserted adjacent and C-terminal to the position indicated, as defined relative to SEQ ID NO:1, in the constant region of its Ig light chain amino acid sequence, was tested (FIG. 3). To determine aggregation, samples were analyzed using analytical size exclusion chromatography (SEC; Tosoh #08541) with a mobile phase of 300 mM NaCl, 25 mM sodium phosphate pH 6.8.

### Example 4: Analysis of Conjugation Efficiency of Antibodies with a Tag in the Light Chain

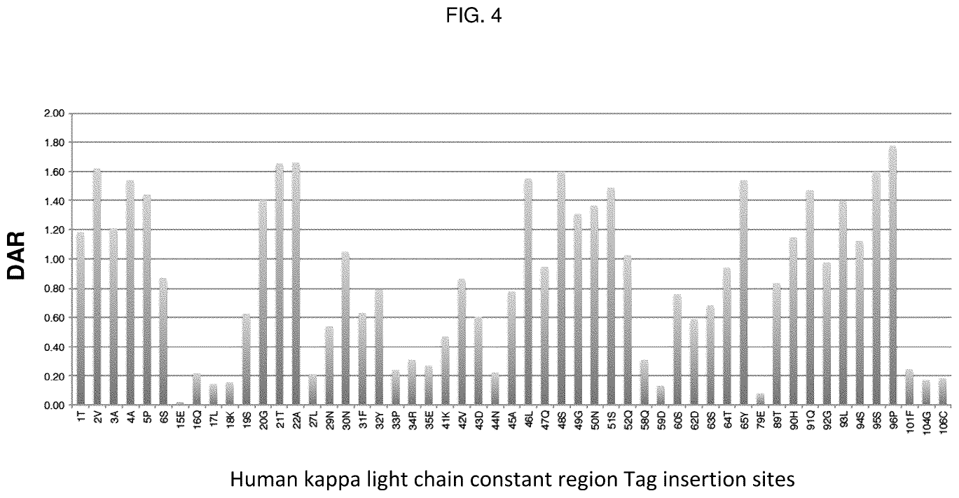

A subset of insertion sites selected based on the titer, as shown in Example 2, was chosen to study the conjugation efficiency, as measured by the drug-to-antibody ratio (DAR). Antibodies having a tagged (fGly-containing) light chain polypeptide were conjugated with a hydrophobic payload, a detectable label which serves as a surrogate for drug. Measurement of DAR of the tagged antibodies after conjugation with the hydrophobic payload showed variable conjugation efficiency across the different insertion sites (FIG. 4).

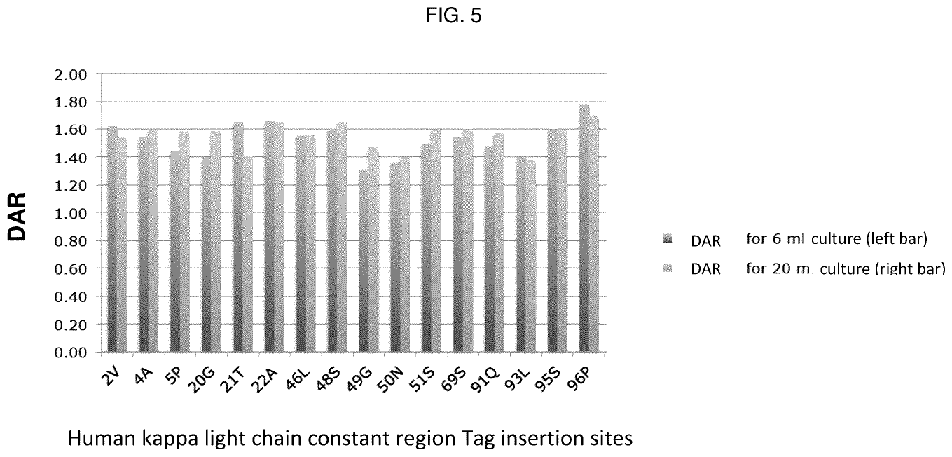

The conjugation yield of light chain-tagged antibodies expressed in ExpiCHO™ cells in a 6 ml culture volume in a tube was similar to the conjugation efficiency when expressed in ExpiCHO™ cells in a 20 ml culture volume in a flask, showing that the conjugation efficiency of the light-chain tagged antibody with a hydrophobic payload was consistent between tagged antibodies produced under different culture conditions (FIG. 5).

### Materials and Methods

To determine the drug-to-antibody ratio (DAR) of the final product, antibody-drug conjugates (ADCs) were examined by analytical HIC (Tosoh #14947) with mobile phase A: 1.5 M ammonium sulfate, 25 mM sodium phosphate pH 7.0, and mobile phase B: 25% isopropanol, 18.75 mM sodium phosphate pH 7.0.

### Example 5: Functional Properties of Antibody-Drug Conjugates

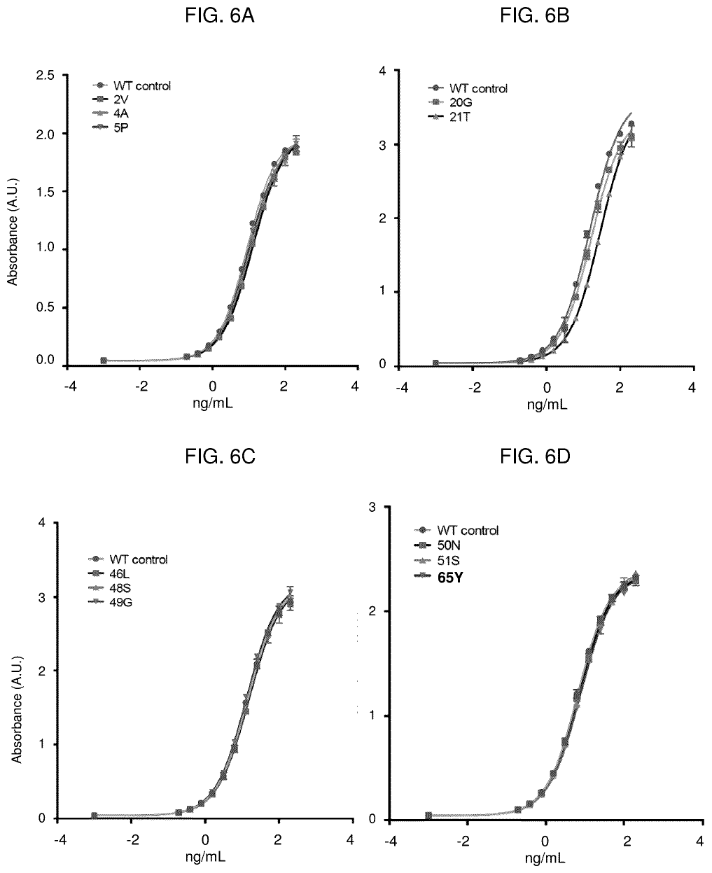

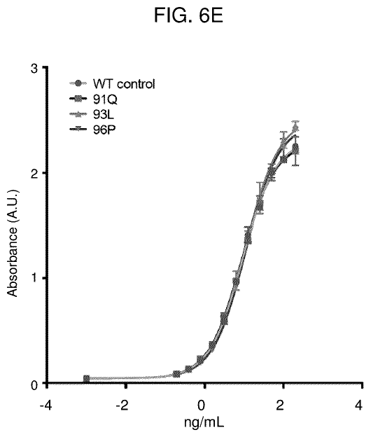

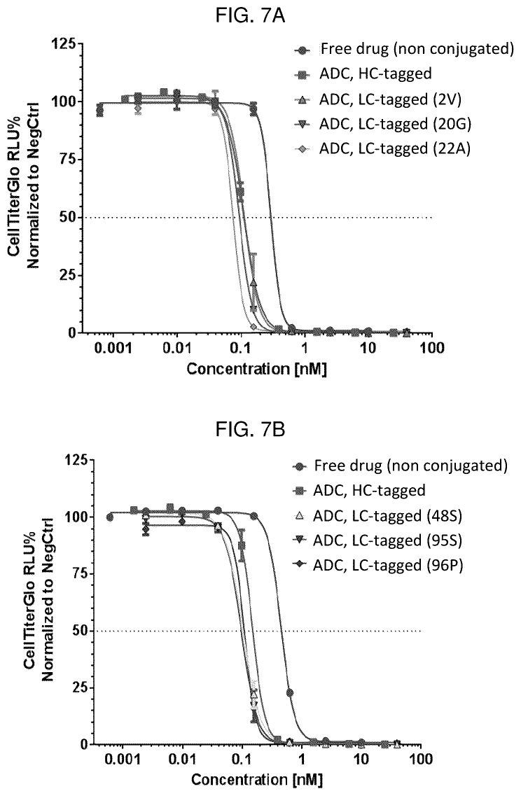

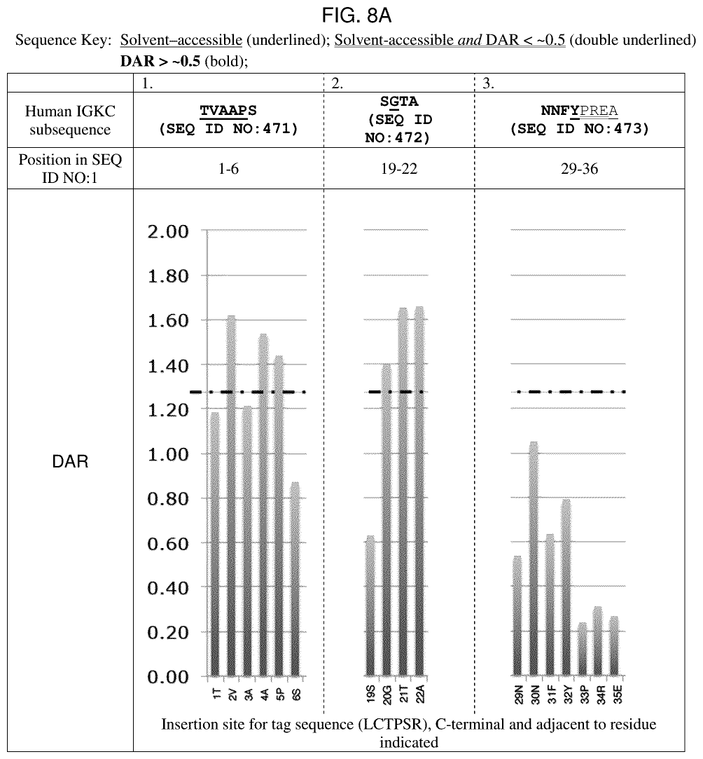

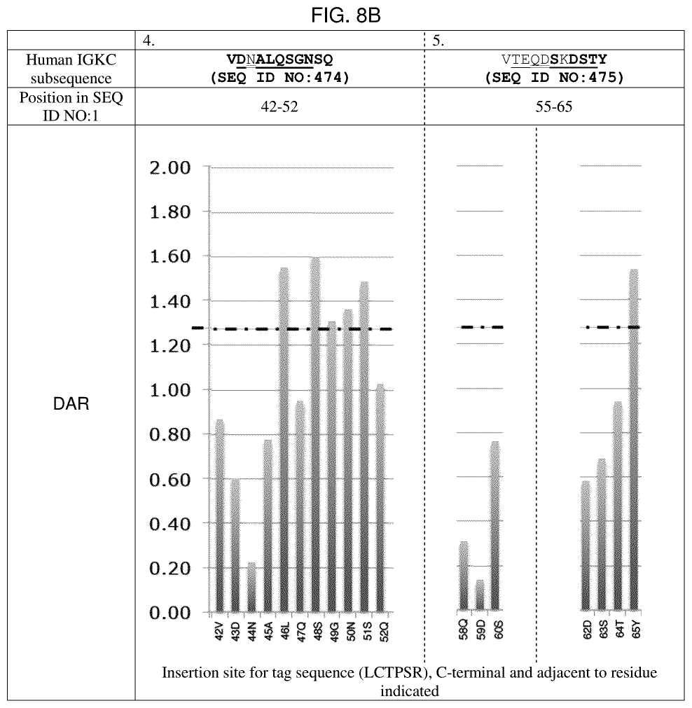

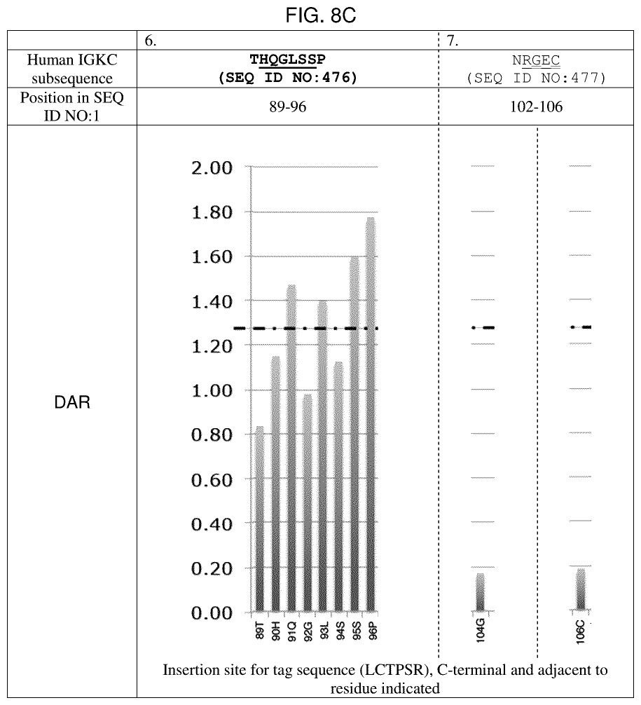

Select light chain-tagged antibody-drug conjugates were tested for antigen binding using ELISA. All light chain-tagged antibody-drug conjugates tested had antigen binding properties similar to the parent antibody (FIGS. 6A-6E).

Select light chain-tagged antibodies conjugated to a cytotoxic drug were tested for cytotoxic activity towards cells expressing the antigen on the cell surface. All light chain-tagged antibody-drug conjugates tested exhibited enhanced cytotoxicity compared to free drug, and similar potency as a heavy chain-tagged antibody-drug conjugate.

### Materials and Methods

Light chain-tagged antibody (1 mg/mL) was conjugated to a hydrophobic payload (cytotoxic drug) functionalized with an aldehyde-reactive group (360 mol. equivalents drug:antibody) for 16-24 h at 37° C. in 50 mM sodium citrate, 50 mM NaCl pH 5.5 containing 0.85% dimethylacetamide (DMA). Unconjugated drug was removed by repeated buffer exchange using a centrifugal concentrator with a molecular weight cutoff (MWCO) of 10 kD.

While the present disclosure has been described with reference to the specific embodiments thereof, it should be understood by those skilled in the art that various changes may be made and equivalents may be substituted without departing from the true spirit and scope of the present disclosure. In addition, many modifications may be made to adapt a particular situation, material, composition of matter, process, process step or steps, to the objective, spirit and scope of the present disclosure. All such modifications are intended to be within the scope of the claims appended hereto.

1.-41. (canceled)

42. An immunoglobulin (Ig) kappa light chain polypeptide comprising, in a constant region, an amino acid sequence selected from:
i) SEQ ID NO: 6, SEQ ID NO: 16, SEQ ID NO: 29, SEQ ID NO: 30, SEQ ID NO: 40, and SEQ ID NO: 42; or
ii) a sequence having, exclusive of the sequence LCTPSR (SEQ ID NO: 158), at least 90% sequence identity to a sequence from entry (i);
wherein the cysteine residue in the sequence LCTPSR (SEQ ID NO: 158) is oxidized to fGly.

43. The Ig kappa light chain polypeptide of claim 42, wherein the fGly is covalently bound to a payload.

44. The Ig kappa light chain polypeptide of claim 43, wherein the fGly is covalently bound to the payload via a hydrazone, oxime, semicarbazone, alkyl, alkenyl, acyloxy, hydrazinyl-indolyl, hydrazinyl-imidazoyl, hydrazinyl-pyrrolyl, hydrazinyl-furanyl or a pyrazolinone linkage.

45. The Ig kappa light chain polypeptide of claim 43, wherein the fGly is covalently bound to the payload via a linking group.

46. The Ig kappa light chain polypeptide of claim 45, wherein the linking group comprises a 4-aminopiperidine derivative (4AP).

47. The Ig kappa light chain polypeptide of claim 43, wherein the payload is a drug, a detectable label, a water-soluble polymer, a viral fusion inhibitor, or a synthetic peptide.

48. The Ig kappa light chain polypeptide of claim 47, wherein the drug is a small molecule drug.

49. The Ig kappa light chain polypeptide of claim 48, wherein the small molecule drug is a cancer chemotherapeutic agent.

50. The Ig kappa light chain polypeptide of claim 49, wherein the cancer chemotherapeutic agent is an alkylating agent, a nitrosourea, an antimetabolite, an antitumor antibiotic, a vinca alkaloid, or a steroid hormone.

51. The Ig kappa light chain polypeptide of claim 47, wherein the water-soluble polymer is poly(ethylene glycol).

52. The Ig kappa light chain polypeptide of claim 47, wherein the detectable label is an imaging agent.

53. A recombinant nucleic acid comprising a nucleotide sequence encoding the Ig kappa light chain polypeptide of claim 42.

54. A recombinant expression vector comprising the nucleic acid of claim 53, wherein the nucleotide sequence encoding the Ig kappa light chain polypeptide is operably linked to a promoter.

55. A host cell genetically modified to express the Ig kappa light chain polypeptide of claim 42.

56. The host cell of claim 55, genetically modified to express a formylglycine generating enzyme (FGE), in a manner sufficient to convert the Ig kappa light chain polypeptide into an fGly-modified Ig kappa light chain polypeptide.

57. The host cell of claim 56, wherein the host cell is a mammalian cell.

58. An antibody comprising the Ig kappa light chain polypeptide of claim 42.

59. The antibody of claim 58, wherein the antibody specifically binds a tumor antigen on a cancer cell.

60. A formulation comprising: the antibody of claim 59 and a pharmaceutically acceptable excipient.

61. An immunoglobulin (Ig) kappa light chain polypeptide comprising, in a constant region, an amino acid sequence selected from the group consisting of: i) SEQ ID NO: 6, SEQ ID NO: 16, SEQ ID NO: 29, SEQ ID NO: 30, SEQ ID NO: 40, and SEQ ID NO: 42,
wherein the cysteine residue in the sequence LCTPSR (SEQ ID NO: 158) is oxidized to fGly.


SEQUENCE LISTING (515 sequences)

SEQ ID NO: 1 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 112 aa, synthetic construct
TLCTPSRVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 112 aa, synthetic construct
TVLCTPSRAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 112 aa, synthetic construct
TVALCTPSRAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 112 aa, synthetic construct
TVAALCTPSRPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 112 aa, synthetic construct
TVAAPLCTPSRSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 112 aa, synthetic construct
TVAAPSLCTPSRVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 112 aa, synthetic construct
TVAAPSVLCTPSRFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 112 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFLCTPSRNRGEC

SEQ ID NO: 10 - protein, 112 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNLCTPSRRGEC

SEQ ID NO: 11 - protein, 112 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRLCTPSRGEC

SEQ ID NO: 12 - protein, 112 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGLCTPSREC

SEQ ID NO: 13 - protein, 112 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGELCTPSRC

SEQ ID NO: 14 - protein, 112 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLCTPSR

SEQ ID NO: 15 - protein, 112 aa, synthetic construct
TVAAPSVFIFPPSDEQLKSLCTPSRGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 112 aa, synthetic construct
TVAAPSVFIFPPSDEQLKSGLCTPSRTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 112 aa, synthetic construct
TVAAPSVFIFPPSDEQLKSGTLCTPSRASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 112 aa, synthetic construct
TVAAPSVFIFPPSDEQLKSGTALCTPSRSVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 112 aa, synthetic construct
TVAAPSVFIFPPSDEQLKSGTASVVCLLNLCTPSRNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 112 aa, synthetic construct
TVAAPSVFIFPPSDEQLKSGTASVVCLLNNLCTPSRFYPREAKVQWKVDNALQSGNSQESVTEQDSKDSTYSLSSTLTLSKADYEKHKVYACEVTHQGLSSPVTKSFNRGEC

SEQ ID NO: 21 - protein, 112 aa, synthetic construct
TVAAPSVFIFPPSDEQLKSGTASVVCLLNNFLCTPSRYPREAKVQWKVDNALQSGNSQESVTEQDSKDSTYSLSSTLTLSKADYEKHKVYACEVTHQGLSSPVTKSFNRGEC

SEQ ID NO: 22 - protein, 112 aa, synthetic construct
TVAAPSVFIFPPSDEQLKSGTASVVCLLNNFYLCTPSRPREAKVQWKVDNALQSGNSQESVTEQDSKDSTYSLSSTLTLSKADYEKHKVYACEVTHQGLSSPVTKSFNRGEC

SEQ ID NO: 23 - protein, 112 aa, synthetic construct
TVAAPSVFIFPPSDEQLKSGTASVVCLLNNFYPREAKVQWKVLCTPSRDNALQSGNSQESVTEQDSKDSTYSLSSTLTLSKADYEKHKVYACEVTHQGLSSPVTKSFNRGEC

SEQ ID NO: 24 - protein, 112 aa, synthetic construct
TVAAPSVFIFPPSDEQLKSGTASVVCLLNNFYPREAKVQWKVDLCTPSRNALQSGNSQESVTEQDSKDSTYSLSSTLTLSKADYEKHKVYACEVTHQGLSSPVTKSFNRGEC

SEQ ID NO: 25 - protein, 112 aa, synthetic construct
TVAAPSVFIFPPSDEQLKSGTASVVCLLNNFYPREAKVQWKVDNALCTPSRLQSGNSQESVTEQDSKDSTYSLSSTLTLSKADYEKHKVYACEVTHQGLSSPVTKSFNRGEC

SEQ ID NO: 26 - protein, 112 aa, synthetic construct
TVAAPSVFIFPPSDEQLKSGTASVVCLLNNFYPREAKVQWKVDNALLCTPSRQSGNSQESVTEQDSKDSTYSLSSTLTLSKADYEKHKVYACEVTHQGLSSPVTKSFNRGEC

SEQ ID NO: 27 - protein, 112 aa, synthetic construct
TVAAPSVFIFPPSDEQLKSGTASVVCLLNNFYPREAKVQWKVDNALQLCTPSRSGNSQESVTEQDSKDSTYSLSSTLTLSKADYEKHKVYACEVTHQGLSSPVTKSFNRGEC

SEQ ID NO: 28 - protein, 112 aa, synthetic construct
TVAAPSVFIFPPSDEQLKSGTASVVCLLNNFYPREAKVQWKVDNALQSLCTPSRGNSQESVTEQDSKDSTYSLSSTLTLSKADYEKHKVYACEVTHQGLSSPVTKSFNRGEC

SEQ ID NO: 29 - protein, 112 aa, synthetic construct
TVAAPSVFIFPPSDEQLKSGTASVVCLLNNFYPREAKVQWKVDNALQSGLCTPSRNSQESVTEQDSKDSTYSLSSTLTLSKADYEKHKVYACEVTHQGLSSPVTKSFNRGEC

SEQ ID NO: 30 - protein, 112 aa, synthetic construct
TVAAPSVFIFPPSDEQLKSGTASVVCLLNNFYPREAKVQWKVDNALQSGNLCTPSRSQESVTEQDSKDSTYSLSSTLTLSKADYEKHKVYACEVTHQGLSSPVTKSFNRGEC

SEQ ID NO: 31 - protein, 112 aa, synthetic construct
TVAAPSVFIFPPSDEQLKSGTASVVCLLNNFYPREAKVQWKVDNALQSGNSLCTPSRQESVTEQDSKDSTYSLSSTLTLSKADYEKHKVYACEVTHQGLSSPVTKSFNRGEC

SEQ ID NO: 32 - protein, 112 aa, synthetic construct
TVAAPSVFIFPPSDEQLKSGTASVVCLLNNFYPREAKVQWKVDNALQSGNSQLCTPSRESVTEQDSKDSTYSLSSTLTLSKADYEKHKVYACEVTHQGLSSPVTKSFNRGEC

SEQ ID NO: 33 - protein, 112 aa, synthetic construct
TVAAPSVFIFPPSDEQLKSGTASVVCLLNNFYPREAKVQWKVDNALQSGNSQESVTEQDSLCTPSRKDSTYSLSSTLTLSKADYEKHKVYACEVTHQGLSSPVTKSFNRGEC

SEQ ID NO: 34 - protein, 112 aa, synthetic construct
TVAAPSVFIFPPSDEQLKSGTASVVCLLNNFYPREAKVQWKVDNALQSGNSQESVTEQDSKDLCTPSRSTYSLSSTLTLSKADYEKHKVYACEVTHQGLSSPVTKSFNRGEC

SEQ ID NO: 35 - protein, 112 aa, synthetic construct
TVAAPSVFIFPPSDEQLKSGTASVVCLLNNFYPREAKVQWKVDNALQSGNSQESVTEQDSKDSLCTPSRTYSLSSTLTLSKADYEKHKVYACEVTHQGLSSPVTKSFNRGEC

SEQ ID NO: 36 - protein, 112 aa, synthetic construct
TVAAPSVFIFPPSDEQLKSGTASVVCLLNNFYPREAKVQWKVDNALQSGNSQESVTEQDSKDSTLCTPSRYSLSSTLTLSKADYEKHKVYACEVTHQGLSSPVTKSFNRGEC

SEQ ID NO: 37 - protein, 112 aa, synthetic construct
TVAAPSVFIFPPSDEQLKSGTASVVCLLNNFYPREAKVQWKVDNALQSGNSQESVTEQDSKDSTYLCTPSRSLSSTLTLSKADYEKHKVYACEVTHQGLSSPVTKSFNRGEC

SEQ ID NO: 38 - protein, 112 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTLCTPSRHQGLSSPVTKSFNRGEC

SEQ ID NO: 39 - protein, 112 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHLCTPSRQGLSSPVTKSFNRGEC

SEQ ID NO: 40 - protein, 112 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQLCTPSRGLSSPVTKSFNRGEC

SEQ ID NO: 41 - protein, 112 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLCTPSRLSSPVTKSFNRGEC

SEQ ID NO: 42 - protein, 112 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLLCTPSRSSPVTKSFNRGEC

SEQ ID NO: 43 - protein, 112 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSLCTPSRSPVTKSFNRGEC

SEQ ID NO: 44 - protein, 112 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSLCTPSRPVTKSFNRGEC

SEQ ID NO: 45 - protein, 112 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPLCTPSRVTKSFNRGEC

SEQ ID NO: 46 - protein, 9 aa, synthetic construct
  VARIANT 2: note = Xaa at position 2 may be present or absent, and when present may be any amino acid
  VARIANT 3: note = Xaa at position 3 is Cys, Ser, 2-formylglycine, or 2-formylglycine covalently bound to a moiety
  VARIANT 4: note = Xaa at position 4 is any amino acid
  VARIANT 5: note = Xaa at position 5 is Pro or Ala
  VARIANT 6: note = Xaa at position 6 is any amino acid
  VARIANT 7: note = Xaa at position 7 may be any aliphatic amino acid or a basic amino acid
TXXXXXRVA

SEQ ID NO: 47 - protein, 10 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
TVXXXXXXAA

SEQ ID NO: 48 - protein, 10 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
VAXXXXXXAP

SEQ ID NO: 49 - protein, 11 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
AAXXXXXXPSV

SEQ ID NO: 50 - protein, 11 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
APXXXXXXSVF

SEQ ID NO: 51 - protein, 10 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
PSXXXXXXVF

SEQ ID NO: 52 - protein, 10 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
KSXXXXXXGT

SEQ ID NO: 53 - protein, 11 aa, synthetic construct
  VARIANT 4: note = Xaa at position 4 may be present or absent, and when present may be any amino acid
  VARIANT 5: note = Xaa at position 5 is Cys, Ser, 2-formylglycine, or 2-formylglycine covalently bound to a moiety
  VARIANT 6: note = Xaa at position 6 is any amino acid
  VARIANT 7: note = Xaa at position 7 is Pro or Ala
  VARIANT 8: note = Xaa at position 8 is any amino acid
  VARIANT 9: note = Xaa at position 9 is an aliphatic amino acid or a basic amino acid
KSGXXXXXXTA

SEQ ID NO: 54 - protein, 10 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
GTXXXXXXAS

SEQ ID NO: 55 - protein, 11 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
TAXXXXXXSVV

SEQ ID NO: 56 - protein, 10 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
LNXXXXXXNF

SEQ ID NO: 57 - protein, 10 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
NNXXXXXXFY

SEQ ID NO: 58 - protein, 10 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
NFXXXXXXYP

SEQ ID NO: 59 - protein, 10 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
FYXXXXXXPR

SEQ ID NO: 60 - protein, 11 aa, synthetic construct
  VARIANT 4: note = Xaa at position 4 may be present or absent, and when present may be any amino acid
  VARIANT 5: note = Xaa at position 5 is Cys, Ser, 2-formylglycine, or 2-formylglycine covalently bound to a moiety
  VARIANT 6: note = Xaa at position 6 is any amino acid
  VARIANT 7: note = Xaa at position 7 is Pro or Ala
  VARIANT 8: note = Xaa at position 8 is any amino acid
  VARIANT 9: note = Xaa at position 9 is an aliphatic amino acid or a basic amino acid
WKVXXXXXXDN

SEQ ID NO: 61 - protein, 10 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 may be any aliphatic amino acid or a basic amino acid
  VARIANT 10: note = Xaa can be Ala or Val
VDXXXXXRNX

SEQ ID NO: 62 - skipped

SEQ ID NO: 63 - protein, 10 aa, synthetic construct
  VARIANT 1: note = Xaa can be Ala or Val
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 may be any aliphatic amino acid or a basic amino acid
XLXXXXXRQS

SEQ ID NO: 64 - protein, 11 aa, synthetic construct
  VARIANT 3..8: note = Xaa can be any naturally occurring amino acid
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
LQXXXXXXSGN

SEQ ID NO: 65 - protein, 10 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
QSXXXXXXGN

SEQ ID NO: 66 - protein, 11 aa, synthetic construct
  VARIANT 4: note = Xaa at position 4 may be present or absent, and when present may be any amino acid
  VARIANT 5: note = Xaa at position 5 is Cys, Ser, 2-formylglycine, or 2-formylglycine covalently bound to a moiety
  VARIANT 6: note = Xaa at position 6 is any amino acid
  VARIANT 7: note = Xaa at position 7 is Pro or Ala
  VARIANT 8: note = Xaa at position 8 is any amino acid
  VARIANT 9: note = Xaa at position 9 is an aliphatic amino acid or a basic amino acid
QSGXXXXXXNS

SEQ ID NO: 67 - protein, 10 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
GNXXXXXXSQ

SEQ ID NO: 68 - protein, 10 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
NSXXXXXXQE

SEQ ID NO: 69 - protein, 10 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
SQXXXXXXES

SEQ ID NO: 70 - protein, 11 aa, synthetic construct
  VARIANT 4: note = Xaa at position 4 may be present or absent, and when present may be any amino acid
  VARIANT 5: note = Xaa at position 5 is Cys, Ser, 2-formylglycine, or 2-formylglycine covalently bound to a moiety
  VARIANT 6: note = Xaa at position 6 is any amino acid
  VARIANT 7: note = Xaa at position 7 is Pro or Ala
  VARIANT 8: note = Xaa at position 8 is any amino acid
  VARIANT 9: note = Xaa at position 9 is an aliphatic amino acid or a basic amino acid
QDSXXXXXXKD

SEQ ID NO: 71 - protein, 11 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
KDXXXXXXSTY

SEQ ID NO: 72 - protein, 11 aa, synthetic construct
  VARIANT 4: note = Xaa at position 4 may be present or absent, and when present may be any amino acid
  VARIANT 5: note = Xaa at position 5 is Cys, Ser, 2-formylglycine, or 2-formylglycine covalently bound to a moiety
  VARIANT 6: note = Xaa at position 6 is any amino acid
  VARIANT 7: note = Xaa at position 7 is Pro or Ala
  VARIANT 8: note = Xaa at position 6 is any amino acid
  VARIANT 9: note = Xaa at position 7 is an aliphatic amino acid or a basic amino acid
KDSXXXXXXTY

SEQ ID NO: 73 - protein, 11 aa, synthetic construct
  VARIANT 4: note = Xaa at position 4 may be present or absent, and when present may be any amino acid
  VARIANT 5: note = Xaa at position 5 is Cys, Ser, 2-formylglycine, or 2-formylglycine covalently bound to a moiety
  VARIANT 6: note = Xaa at position 6 is any amino acid
  VARIANT 7: note = Xaa at position 7 is Pro or Ala
  VARIANT 8: note = Xaa at position 8 is any amino acid
  VARIANT 9: note = Xaa at position 9 is an aliphatic amino acid or a basic amino acid
DSTXXXXXXYS

SEQ ID NO: 74 - protein, 10 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
TYXXXXXXSL

SEQ ID NO: 75 - protein, 11 aa, synthetic construct
  VARIANT 4: note = Xaa at position 4 may be present or absent, and when present may be any amino acid
  VARIANT 5: note = Xaa at position 5 is Cys, Ser, 2-formylglycine, or 2-formylglycine covalently bound to a moiety
  VARIANT 6: note = Xaa at position 6 is any amino acid
  VARIANT 7: note = Xaa at position 7 is Pro or Ala
  VARIANT 8: note = Xaa at position 8 is any amino acid
  VARIANT 9: note = Xaa at position 9 is an aliphatic amino acid or a basic amino acid
EVTXXXXXXHQ

SEQ ID NO: 76 - protein, 10 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
THXXXXXXQG

SEQ ID NO: 77 - protein, 10 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
HQXXXXXXGL

SEQ ID NO: 78 - protein, 12 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
QGXXXXXXLSSP

SEQ ID NO: 79 - protein, 10 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
GLXXXXXXSS

SEQ ID NO: 80 - protein, 11 aa, synthetic construct
  VARIANT 4: note = Xaa at position 4 may be present or absent, and when present may be any amino acid
  VARIANT 5: note = Xaa at position 5 is Cys, Ser, 2-formylglycine, or 2-formylglycine covalently bound to a moiety
  VARIANT 6: note = Xaa at position 6 is any amino acid
  VARIANT 7: note = Xaa at position 7 is Pro or Ala
  VARIANT 8: note = Xaa at position 8 is any amino acid
  VARIANT 9: note = Xaa at position 9 is an aliphatic amino acid or a basic amino acid
GLSXXXXXXSP

SEQ ID NO: 81 - protein, 12 aa, synthetic construct
  VARIANT 5: note = Xaa at position 5 may be present or absent, and when present may be any amino acid
  VARIANT 6: note = Xaa at position 6 is Cys, Ser, 2-formylglycine, or 2-formylglycine covalently bound to a moiety
  VARIANT 7: note = Xaa at position 7 is any amino acid
  VARIANT 8: note = Xaa at position 8 is Pro or Ala
  VARIANT 9: note = Xaa at position 9 is any amino acid
  VARIANT 10: note = Xaa at position 10 is an aliphatic amino acid or a basic amino acid
GLSSXXXXXXPV

SEQ ID NO: 82 - protein, 10 aa, synthetic construct
  VARIANT 3: note = Xaa at position 3 may be present or absent, and when present may be any amino acid
  VARIANT 4: note = Xaa at position 4 is Cys, Ser, 2-formylglycine, or 2-formylglycine covalently bound to a moiety
  VARIANT 5: note = Xaa at position 5 is any amino acid
  VARIANT 6: note = Xaa at position 6 is Pro or Ala
  VARIANT 7: note = Xaa at position 7 is any amino acid
  VARIANT 8: note = Xaa at position 8 is an aliphatic amino acid or a basic amino acid
SPXXXXXXVT

SEQ ID NO: 83 - protein, 9 aa, synthetic construct
TLCTPSRVA

SEQ ID NO: 84 - protein, 10 aa, synthetic construct
TVLCTPSRAA

SEQ ID NO: 85 - protein, 10 aa, synthetic construct
VALCTPSRAP

SEQ ID NO: 86 - protein, 11 aa, synthetic construct
AALCTPSRPSV

SEQ ID NO: 87 - protein, 11 aa, synthetic construct
APLCTPSRSVF

SEQ ID NO: 88 - protein, 10 aa, synthetic construct
PSLCTPSRVF

SEQ ID NO: 89 - protein, 10 aa, synthetic construct
KSLCTPSRGT

SEQ ID NO: 90 - protein, 11 aa, synthetic construct
KSGLCTPSRTA

SEQ ID NO: 91 - protein, 10 aa, synthetic construct
GTLCTPSRAS

SEQ ID NO: 92 - protein, 11 aa, synthetic construct
TALCTPSRSVV

SEQ ID NO: 93 - protein, 10 aa, synthetic construct
LNLCTPSRNF

SEQ ID NO: 94 - protein, 10 aa, synthetic construct
NNLCTPSRFY

SEQ ID NO: 95 - protein, 10 aa, synthetic construct
NFLCTPSRYP

SEQ ID NO: 96 - protein, 10 aa, synthetic construct
FYLCTPSRPR

SEQ ID NO: 97 - protein, 11 aa, synthetic construct
WKVLCTPSRDN

SEQ ID NO: 98 - protein, 10 aa, synthetic construct
  VARIANT 10: note = Xaa can be Ala or Val
VDLCTPSRNX

SEQ ID NO: 99 - protein, 10 aa, synthetic construct
  VARIANT 2: note = Xaa can be Ala or Val
NXLCTPSRLQ

SEQ ID NO: 100 - protein, 10 aa, synthetic construct
  VARIANT 1: note = Xaa can be Ala or Val
XLLCTPSRQS

SEQ ID NO: 101 - protein, 11 aa, synthetic construct
LQLCTPSRSGN

SEQ ID NO: 102 - protein, 10 aa, synthetic construct
QSLCTPSRGN

SEQ ID NO: 103 - protein, 11 aa, synthetic construct
QSGLCTPSRNS

SEQ ID NO: 104 - protein, 10 aa, synthetic construct
GNLCTPSRSQ

SEQ ID NO: 105 - protein, 10 aa, synthetic construct
NSLCTPSRQE

SEQ ID NO: 106 - protein, 10 aa, synthetic construct
SQLCTPSRES

SEQ ID NO: 107 - protein, 11 aa, synthetic construct
QDSLCTPSRKD

SEQ ID NO: 108 - protein, 11 aa, synthetic construct
KDLCTPSRSTY

SEQ ID NO: 109 - protein, 11 aa, synthetic construct
KDSLCTPSRTY

SEQ ID NO: 110 - protein, 11 aa, synthetic construct
DSTLCTPSRYS

SEQ ID NO: 111 - protein, 10 aa, synthetic construct
TYLCTPSRSL

SEQ ID NO: 112 - protein, 11 aa, synthetic construct
EVTLCTPSRHQ

SEQ ID NO: 113 - protein, 10 aa, synthetic construct
THLCTPSRQG

SEQ ID NO: 114 - protein, 10 aa, synthetic construct
HQLCTPSRGL

SEQ ID NO: 115 - protein, 12 aa, synthetic construct
QGLCTPSRLSSP

SEQ ID NO: 116 - protein, 10 aa, synthetic construct
GLLCTPSRSS

SEQ ID NO: 117 - protein, 11 aa, synthetic construct
GLSLCTPSRSP

SEQ ID NO: 118 - protein, 12 aa, synthetic construct
GLSSLCTPSRPV

SEQ ID NO: 119 - protein, 10 aa, synthetic construct
SPLCTPSRVT

SEQ ID NO: 120 - protein, 9 aa, synthetic construct
  VARIANT 2: note = Xaa at position 2 may be present or absent, and when present may be any amino acid
  MOD_RES 3: note = FORMYLATION, covalently bound to a moiety
  VARIANT 4: note = Xaa can be any naturally occurring amino acid
  VARIANT 5: note = Xaa at position 5 is Pro or Ala
  VARIANT 6: note = Xaa can be any naturally occurring amino acid
  VARIANT 7: note = Xaa at position 7 is an aliphatic or baisc amino acid
TXGXXXXVA

SEQ ID NO: 121 - protein, 10 aa, synthetic construct
  VARIANT 3: note = Xaa can be any naturally occurring amino acid
  MOD_RES 4: note = FORMYLATION, covalently bound to a moiety
  VARIANT 5..8: note = Xaa can be any naturally occurring amino acid
TVXGXXXXAA

SEQ ID NO: 122 - protein, 10 aa, synthetic construct
  VARIANT 3: note = Xaa can be any naturally occurring amino acid
  MOD_RES 4: note = FORMYLATION, covalently bound to a moiety
  VARIANT 5..8: note = Xaa can be any naturally occurring amino acid
VAXGXXXXAP

SEQ ID NO: 123 - protein, 11 aa, synthetic construct
  VARIANT 3: note = Xaa can be any naturally occurring amino acid
  MOD_RES 4: note = FORMYLATION, covalently bound to a moiety
  VARIANT 5..8: note = Xaa can be any naturally occurring amino acid
AAXGXXXXPSV

SEQ ID NO: 124 - protein, 11 aa, synthetic construct
  VARIANT 3: note = Xaa can be any naturally occurring amino acid
  MOD_RES 4: note = FORMYLATION, covalently bound to a moiety
  VARIANT 5..8: note = Xaa can be any naturally occurring amino acid
APXGXXXXSVF

SEQ ID NO: 125 - protein, 10 aa, synthetic construct
  VARIANT 3: note = Xaa can be any naturally occurring amino acid
  MOD_RES 4: note = FORMYLATION, covalently bound to a moiety
  VARIANT 5..8: note = Xaa can be any naturally occurring amino acid
PSXGXXXXVF

SEQ ID NO: 126 - protein, 10 aa, synthetic construct
  VARIANT 3: note = Xaa can be any naturally occurring amino acid
  MOD_RES 4: note = FORMYLATION, covalently bound to a moiety
  VARIANT 5..8: note = Xaa can be any naturally occurring amino acid
KSXGXXXXGT

SEQ ID NO: 127 - protein, 11 aa, synthetic construct
  VARIANT 4: note = Xaa can be any naturally occurring amino acid
  MOD_RES 5: note = FORMYLATION, covalently bound to a moiety
  VARIANT 6..9: note = Xaa can be any naturally occurring amino acid
KSGXGXXXXTA

SEQ ID NO: 128 - protein, 10 aa, synthetic construct
  VARIANT 3: note = Xaa can be any naturally occurring amino acid
  MOD_RES 4: note = FORMYLATION, covalently bound to a moiety
  VARIANT 5..8: note = Xaa can be any naturally occurring amino acid
GTXGXXXXAS

SEQ ID NO: 129 - protein, 11 aa, synthetic construct
  VARIANT 3: note = Xaa can be any naturally occurring amino acid
  MOD_RES 4: note = FORMYLATION, covalently bound to a moiety
  VARIANT 5..8: note = Xaa can be any naturally occurring amino acid
TAXGXXXXSVV

SEQ ID NO: 130 - protein, 10 aa, synthetic construct
  VARIANT 3: note = Xaa can be any naturally occurring amino acid
  MOD_RES 4: note = FORMYLATION, covalently bound to a moiety
  VARIANT 5..8: note = Xaa can be any naturally occurring amino acid
LNXGXXXXNF

SEQ ID NO: 131 - protein, 10 aa, synthetic construct
  VARIANT 3: note = Xaa can be any naturally occurring amino acid
  MOD_RES 4: note = , covalently bound to a moiety
  VARIANT 5..8: note = Xaa can be any naturally occurring amino acid
NNXGXXXXFY

SEQ ID NO: 132 - protein, 10 aa, synthetic construct
  VARIANT 3: note = Xaa can be any naturally occurring amino acid
  MOD_RES 4: note = , covalently bound to a moiety
  VARIANT 5..8: note = Xaa can be any naturally occurring amino acid
NFXGXXXXYP

SEQ ID NO: 133 - protein, 10 aa, synthetic construct
  VARIANT 3: note = Xaa can be any naturally occurring amino acid
  MOD_RES 4: note = , covalently bound to a moiety
  VARIANT 5..8: note = Xaa can be any naturally occurring amino acid
FYXGXXXXPR

SEQ ID NO: 134 - protein, 11 aa, synthetic construct
  VARIANT 4: note = Xaa can be any naturally occurring amino acid
  MOD_RES 5: note = , covalently bound to a moiety
  VARIANT 6..9: note = Xaa can be any naturally occurring amino acid
WKVXGXXXXDN

SEQ ID NO: 135 - protein, 10 aa, synthetic construct
  VARIANT 3: note = Xaa can be any naturally occurring amino acid
  MOD_RES 4: note = , covalently bound to a moiety
  VARIANT 5..8: note = Xaa can be any naturally occurring amino acid
  VARIANT 10: note = Xaa can be Ala or Val
VDXGXXXXNX

SEQ ID NO: 136 - protein, 10 aa, synthetic construct
  VARIANT 2: note = Xaa can be Ala or Val
  MOD_RES 4: note = , covalently bound to a moiety
  VARIANT 5..8: note = Xaa can be any naturally occurring amino acid
NXGXXXXXLQ

SEQ ID NO: 137 - protein, 10 aa, synthetic construct
  VARIANT 1: note = Xaa can be Ala or Val
  VARIANT 3: note = Xaa can be any naturally occurring amino acid
  MOD_RES 4: note = , covalently bound to a moiety
  VARIANT 5..8: note = Xaa can be any naturally occurring amino acid
XLXGXXXXQS

SEQ ID NO: 138 - protein, 11 aa, synthetic construct
  VARIANT 3: note = Xaa can be any naturally occurring amino acid
  MOD_RES 4: note = , covalently bound to a moiety
  VARIANT 5..8: note = Xaa can be any naturally occurring amino acid
LQXGXXXXSGN

SEQ ID NO: 139 - protein, 10 aa, synthetic construct
  VARIANT 3: note = Xaa can be any naturally occurring amino acid
  MOD_RES 4: note = , covalently bound to a moiety
  VARIANT 5..8: note = Xaa can be any naturally occurring amino acid
QSXGXXXXGN

SEQ ID NO: 140 - protein, 11 aa, synthetic construct
  VARIANT 4: note = Xaa can be any naturally occurring amino acid
  MOD_RES 5: note = , covalently bound to a moiety
  VARIANT 6..9: note = Xaa can be any naturally occurring amino acid
QSGXGXXXXNS

SEQ ID NO: 141 - protein, 10 aa, synthetic construct
  VARIANT 3: note = Xaa can be any naturally occurring amino acid
  MOD_RES 4: note = , covalently bound to a moiety
  VARIANT 5..8: note = Xaa can be any naturally occurring amino acid
GNXGXXXXSQ

SEQ ID NO: 142 - protein, 10 aa, synthetic construct
  VARIANT 3: note = Xaa can be any naturally occurring amino acid
  MOD_RES 4: note = , covalently bound to a moiety
  VARIANT 5..8: note = Xaa can be any naturally occurring amino acid
NSXGXXXXQE

SEQ ID NO: 143 - protein, 10 aa, synthetic construct
  VARIANT 3: note = Xaa can be any naturally occurring amino acid
  MOD_RES 4: note = , covalently bound to a moiety
  VARIANT 5..8: note = Xaa can be any naturally occurring amino acid
SQXGXXXXES

SEQ ID NO: 144 - protein, 11 aa, synthetic construct
  VARIANT 4: note = Xaa can be any naturally occurring amino acid
  MOD_RES 5: note = , covalently bound to a moiety
  VARIANT 6..9: note = Xaa can be any naturally occurring amino acid
QDSXGXXXXKD

SEQ ID NO: 145 - protein, 11 aa, synthetic construct
  VARIANT 3: note = Xaa can be any naturally occurring amino acid
  MOD_RES 4: note = , covalently bound to a moiety
  VARIANT 5..8: note = Xaa can be any naturally occurring amino acid
KDXGXXXXSTY

SEQ ID NO: 146 - protein, 11 aa, synthetic construct
  VARIANT 4: note = Xaa can be any naturally occurring amino acid
  MOD_RES 5: note = , covalently bound to a moiety
  VARIANT 6..9: note = Xaa can be any naturally occurring amino acid
KDSXGXXXXTY

SEQ ID NO: 147 - protein, 11 aa, synthetic construct
  VARIANT 4: note = Xaa can be any naturally occurring amino acid
  MOD_RES 5: note = , covalently bound to a moiety
  VARIANT 6..9: note = Xaa can be any naturally occurring amino acid
DSTXGXXXXYS

SEQ ID NO: 148 - protein, 10 aa, synthetic construct
  VARIANT 3: note = Xaa can be any naturally occurring amino acid
  MOD_RES 4: note = , covalently bound to a moiety
  VARIANT 5..8: note = Xaa can be any naturally occurring amino acid
TYXGXXXXSL

SEQ ID NO: 149 - protein, 11 aa, synthetic construct
  VARIANT 4: note = Xaa can be any naturally occurring amino acid
  MOD_RES 5: note = FORMYLATION, covalently bound to a moiety
  VARIANT 6..9: note = Xaa can be any naturally occurring amino acid
EVTXGXXXXHQ

SEQ ID NO: 150 - protein, 10 aa, synthetic construct
  VARIANT 3: note = Xaa can be any naturally occurring amino acid
  MOD_RES 4: note = FORMYLATION, covalently bound to a moiety
  VARIANT 5..8: note = Xaa can be any naturally occurring amino acid
THXGXXXXQG

SEQ ID NO: 151 - protein, 10 aa, synthetic construct
  VARIANT 3: note = Xaa can be any naturally occurring amino acid
  MOD_RES 4: note = FORMYLATION, covalently bound to a moiety
  VARIANT 5..8: note = Xaa can be any naturally occurring amino acid
HQXGXXXXGL

SEQ ID NO: 152 - protein, 12 aa, synthetic construct
  VARIANT 3: note = Xaa can be any naturally occurring amino acid
  MOD_RES 4: note = FORMYLATION, covalently bound to a moiety
  VARIANT 5..8: note = Xaa can be any naturally occurring amino acid
QGXGXXXXLSSP

SEQ ID NO: 153 - protein, 10 aa, synthetic construct
  VARIANT 3: note = Xaa can be any naturally occurring amino acid
  MOD_RES 4: note = FORMYLATION, covalently bound to a moiety
  VARIANT 5..8: note = Xaa can be any naturally occurring amino acid
GLXGXXXXSS

SEQ ID NO: 154 - protein, 11 aa, synthetic construct
  VARIANT 4: note = Xaa can be any naturally occurring amino acid
  MOD_RES 5: note = FORMYLATION, covalently bound to a moiety
  VARIANT 6..9: note = Xaa can be any naturally occurring amino acid
GLSXGXXXXSP

SEQ ID NO: 155 - protein, 12 aa, synthetic construct
  VARIANT 5: note = Xaa can be any naturally occurring amino acid
  MOD_RES 6: note = FORMYLATION, covalently bound to a moiety
  VARIANT 7..10: note = Xaa can be any naturally occurring amino acid
GLSSXGXXXXPV

SEQ ID NO: 156 - protein, 10 aa, synthetic construct
  VARIANT 3: note = Xaa can be any naturally occurring amino acid
  MOD_RES 4: note = FORMYLATION, covalently bound to a moiety
  VARIANT 5..8: note = Xaa can be any naturally occurring amino acid
SPXGXXXXVT

SEQ ID NO: 157 - protein, 6 aa, synthetic construct
  MOD_RES 2: note = FORMYLATION
LGTPSR

SEQ ID NO: 158 - protein, 6 aa, synthetic construct
LCTPSR

SEQ ID NO: 159 - protein, 6 aa, synthetic construct
LSTPSR

SEQ ID NO: 160 - protein, 6 aa, synthetic construct
MCTPSR

SEQ ID NO: 161 - protein, 6 aa, synthetic construct
VCTPSR

SEQ ID NO: 162 - protein, 6 aa, synthetic construct
LCSPSR

SEQ ID NO: 163 - protein, 6 aa, synthetic construct
LCAPSR

SEQ ID NO: 164 - protein, 6 aa, synthetic construct
LCVPSR

SEQ ID NO: 165 - protein, 6 aa, synthetic construct
LCGPSR

SEQ ID NO: 166 - protein, 6 aa, synthetic construct
ICTPAR

SEQ ID NO: 167 - protein, 6 aa, synthetic construct
LCTPSK

SEQ ID NO: 168 - protein, 6 aa, synthetic construct
MCTPSK

SEQ ID NO: 169 - protein, 6 aa, synthetic construct
VCTPSK

SEQ ID NO: 170 - protein, 6 aa, synthetic construct
LCSPSK

SEQ ID NO: 171 - protein, 6 aa, synthetic construct
LCAPSK

SEQ ID NO: 172 - protein, 6 aa, synthetic construct
LCVPSK

SEQ ID NO: 173 - protein, 6 aa, synthetic construct
LCGPSK

SEQ ID NO: 174 - protein, 6 aa, synthetic construct
LCTPSA

SEQ ID NO: 175 - protein, 6 aa, synthetic construct
ICTPAA

SEQ ID NO: 176 - protein, 6 aa, synthetic construct
MCTPSA

SEQ ID NO: 177 - protein, 6 aa, synthetic construct
VCTPSA

SEQ ID NO: 178 - protein, 6 aa, synthetic construct
LCSPSA

SEQ ID NO: 179 - protein, 6 aa, synthetic construct
LCAPSA

SEQ ID NO: 180 - protein, 6 aa, synthetic construct
LCVPSA

SEQ ID NO: 181 - protein, 6 aa, synthetic construct
LCGPSA

SEQ ID NO: 182 - protein, 6 aa, synthetic construct
MSTPSR

SEQ ID NO: 183 - protein, 6 aa, synthetic construct
VSTPSR

SEQ ID NO: 184 - protein, 6 aa, synthetic construct
LSSPSR

SEQ ID NO: 185 - protein, 6 aa, synthetic construct
LSAPSR

SEQ ID NO: 186 - protein, 6 aa, synthetic construct
LSVPSR

SEQ ID NO: 187 - protein, 6 aa, synthetic construct
LSGPSR

SEQ ID NO: 188 - protein, 6 aa, synthetic construct
ISTPAR

SEQ ID NO: 189 - protein, 6 aa, synthetic construct
LSTPSK

SEQ ID NO: 190 - protein, 6 aa, synthetic construct
MSTPSK

SEQ ID NO: 191 - protein, 6 aa, synthetic construct
VSTPSK

SEQ ID NO: 192 - protein, 6 aa, synthetic construct
LSSPSK

SEQ ID NO: 193 - protein, 6 aa, synthetic construct
LSAPSK

SEQ ID NO: 194 - protein, 6 aa, synthetic construct
LSVPSK

SEQ ID NO: 195 - protein, 6 aa, synthetic construct
LSGPSK

SEQ ID NO: 196 - protein, 6 aa, synthetic construct
LSTPSA

SEQ ID NO: 197 - protein, 6 aa, synthetic construct
ISTPAA

SEQ ID NO: 198 - protein, 6 aa, synthetic construct
MSTPSA

SEQ ID NO: 199 - protein, 6 aa, synthetic construct
VSTPSA

SEQ ID NO: 200 - protein, 6 aa, synthetic construct
LSSPSA

SEQ ID NO: 201 - protein, 6 aa, synthetic construct
LSAPSA

SEQ ID NO: 202 - protein, 6 aa, synthetic construct
LSVPSA

SEQ ID NO: 203 - protein, 6 aa, synthetic construct
LSGPSA

SEQ ID NO: 204 - protein, 6 aa, synthetic construct
  MOD_RES 2: note = FORMYLATION
MGTPSR

SEQ ID NO: 205 - protein, 6 aa, synthetic construct
  MOD_RES 2: note = FORMYLATION
VGTPSR

SEQ ID NO: 206 - protein, 6 aa, synthetic construct
  MOD_RES 2: note = FORMYLATION
LGSPSR

SEQ ID NO: 207 - protein, 6 aa, synthetic construct
  MOD_RES 2: note = FORMYLATION
LGAPSR

SEQ ID NO: 208 - protein, 6 aa, synthetic construct
  MOD_RES 2: note = FORMYLATION
LGVPSR

SEQ ID NO: 209 - protein, 6 aa, synthetic construct
  MOD_RES 2: note = FORMYLATION
LGGPSR

SEQ ID NO: 210 - protein, 6 aa, synthetic construct
  MOD_RES 2: note = FORMYLATION
IGTPAR

SEQ ID NO: 211 - protein, 6 aa, synthetic construct
  MOD_RES 2: note = FORMYLATION
LGTPSK

SEQ ID NO: 212 - protein, 6 aa, synthetic construct
  MOD_RES 2: note = FORMYLATION
MGTPSK

SEQ ID NO: 213 - protein, 6 aa, synthetic construct
  MOD_RES 2: note = FORMYLATION
VGTPSK

SEQ ID NO: 214 - protein, 6 aa, synthetic construct
  MOD_RES 2: note = FORMYLATION
LGSPSK

SEQ ID NO: 215 - protein, 6 aa, synthetic construct
  MOD_RES 2: note = FORMYLATION
LGAPSK

SEQ ID NO: 216 - protein, 6 aa, synthetic construct
  MOD_RES 2: note = FORMYLATION
LGVPSK

SEQ ID NO: 217 - protein, 6 aa, synthetic construct
  MOD_RES 2: note = FORMYLATION
LGGPSK

SEQ ID NO: 218 - protein, 6 aa, synthetic construct
  MOD_RES 2: note = FORMYLATION
LGTPSA

SEQ ID NO: 219 - protein, 6 aa, synthetic construct
  MOD_RES 2: note = FORMYLATION
IGTPAA

SEQ ID NO: 220 - protein, 6 aa, synthetic construct
  MOD_RES 2: note = FORMYLATION
MGTPSA

SEQ ID NO: 221 - protein, 6 aa, synthetic construct
  MOD_RES 2: note = FORMYLATION
VGTPSA

SEQ ID NO: 222 - protein, 6 aa, synthetic construct
  MOD_RES 2: note = FORMYLATION
LGSPSA

SEQ ID NO: 223 - protein, 6 aa, synthetic construct
  MOD_RES 2: note = FORMYLATION
LGAPSA

SEQ ID NO: 224 - protein, 6 aa, synthetic construct
  MOD_RES 2: note = FORMYLATION
LGVPSA

SEQ ID NO: 225 - protein, 6 aa, synthetic construct
  MOD_RES 2: note = FORMYLATION
LGGPSA

SEQ ID NO: 226 - protein, 6 aa, synthetic construct
  MOD_RES 2: note = FORMYLATION, covalently bound to a moiety
LGTPSR

SEQ ID NO: 227 - protein, 6 aa, synthetic construct
  MOD_RES 2: note = FORMYLATION, covalently bound to a moiety
MGTPSR

SEQ ID NO: 228 - protein, 6 aa, synthetic construct
  MOD_RES 2: note = FORMYLATION, covalently bound to a moiety
VGTPSR

SEQ ID NO: 229 - protein, 6 aa, synthetic construct
  MOD_RES 2: note = FORMYLATION, covalently bound to a moiety
LGSPSR

SEQ ID NO: 230 - protein, 6 aa, synthetic construct
  MOD_RES 2: note = FORMYLATION, covalently bound to a moiety
LGAPSR

SEQ ID NO: 231 - protein, 6 aa, synthetic construct
  MOD_RES 2: note = FORMYLATION, covalently bound to a moiety
LGVPSR

SEQ ID NO: 232 - protein, 6 aa, synthetic construct
  MOD_RES 2: note = FORMYLATION, covalently bound to a moiety
LGGPSR

SEQ ID NO: 233 - protein, 6 aa, synthetic construct
  MOD_RES 2: note = FORMYLATION, covalently bound to a moiety
IGTPAR

SEQ ID NO: 234 - protein, 6 aa, synthetic construct
  MOD_RES 2: note = FORMYLATION, covalently bound to a moiety
LGTPSK

SEQ ID NO: 235 - protein, 6 aa, synthetic construct
  MOD_RES 2: note = FORMYLATION, covalently bound to a moiety
MGTPSK

SEQ ID NO: 236 - protein, 6 aa, synthetic construct
  MOD_RES 2: note = FORMYLATION, covalently bound to a moiety
VGTPSK

SEQ ID NO: 237 - protein, 6 aa, synthetic construct
  MOD_RES 2: note = FORMYLATION, covalently bound to a moiety
LGSPSK

SEQ ID NO: 238 - protein, 6 aa, synthetic construct
  MOD_RES 2: note = FORMYLATION, covalently bound to a moiety
LGAPSK

SEQ ID NO: 239 - protein, 6 aa, synthetic construct
  MOD_RES 2: note = FORMYLATION, covalently bound to a moiety
LGVPSK

SEQ ID NO: 240 - protein, 6 aa, synthetic construct
  MOD_RES 2: note = FORMYLATION, covalently bound to a moiety
LGGPSK

SEQ ID NO: 241 - protein, 6 aa, synthetic construct
  MOD_RES 2: note = FORMYLATION, covalently bound to a moiety
LGTPSA

SEQ ID NO: 242 - protein, 6 aa, synthetic construct
  MOD_RES 2: note = FORMYLATION, covalently bound to a moiety
IGTPAA

SEQ ID NO: 243 - protein, 6 aa, synthetic construct
  MOD_RES 2: note = FORMYLATION, covalently bound to a moiety
MGTPSA

SEQ ID NO: 244 - protein, 6 aa, synthetic construct
  MOD_RES 2: note = FORMYLATION, covalently bound to a moiety
VGTPSA

SEQ ID NO: 245 - protein, 6 aa, synthetic construct
  MOD_RES 2: note = FORMYLATION, covalently bound to a moiety
LGSPSA

SEQ ID NO: 246 - protein, 6 aa, synthetic construct
  MOD_RES 2: note = FORMYLATION, covalently bound to a moiety
LGAPSA

SEQ ID NO: 247 - protein, 6 aa, synthetic construct
  MOD_RES 2: note = FORMYLATION, covalently bound to a moiety
LGVPSA

SEQ ID NO: 248 - protein, 6 aa, synthetic construct
  MOD_RES 2: note = FORMYLATION, covalently bound to a moiety
LGGPSA

SEQ ID NO: 249 - protein, 8 aa, synthetic construct
DYKDDDDK

SEQ ID NO: 250 - DNA, 26 bp, synthetic construct
  source 1..26: mol_type = other DNA
tcaaacgtgagtagaatttaaacttt

SEQ ID NO: 251 - DNA, 25 bp, synthetic construct
  source 1..25: mol_type = other DNA
aaagggcgaaaaaccgtctatcagg

SEQ ID NO: 252 - DNA, 43 bp, synthetic construct
  source 1..43: mol_type = other DNA
ctgtgtaccccttccagggtggctgcaccatctgtcttcatct

SEQ ID NO: 253 - DNA, 43 bp, synthetic construct
  source 1..43: mol_type = other DNA
ctgtgtaccccttccagggctgcaccatctgtcttcatcttcc

SEQ ID NO: 254 - DNA, 43 bp, synthetic construct
  source 1..43: mol_type = other DNA
ctgtgtaccccttccagggcaccatctgtcttcatcttcccgc

SEQ ID NO: 255 - DNA, 43 bp, synthetic construct
  source 1..43: mol_type = other DNA
ctgtgtaccccttccaggccatctgtcttcatcttcccgccat

SEQ ID NO: 256 - DNA, 43 bp, synthetic construct
  source 1..43: mol_type = other DNA
ctgtgtaccccttccaggtctgtcttcatcttcccgccatctg

SEQ ID NO: 257 - DNA, 43 bp, synthetic construct
  source 1..43: mol_type = other DNA
ctgtgtaccccttccagggtcttcatcttcccgccatctgatg

SEQ ID NO: 258 - DNA, 43 bp, synthetic construct
  source 1..43: mol_type = other DNA
ctgtgtaccccttccaggttcatcttcccgccatctgatgagc

SEQ ID NO: 259 - DNA, 43 bp, synthetic construct
  source 1..43: mol_type = other DNA
ctgtgtaccccttccaggatcttcccgccatctgatgagcagt

SEQ ID NO: 260 - DNA, 43 bp, synthetic construct
  source 1..43: mol_type = other DNA
ctgtgtaccccttccaggttcccgccatctgatgagcagttga

SEQ ID NO: 261 - DNA, 43 bp, synthetic construct
  source 1..43: mol_type = other DNA
ctgtgtaccccttccaggccgccatctgatgagcagttgaaat

SEQ ID NO: 262 - DNA, 43 bp, synthetic construct
  source 1..43: mol_type = other DNA
ctgtgtaccccttccaggccatctgatgagcagttgaaatctg

SEQ ID NO: 263 - DNA, 43 bp, synthetic construct
  source 1..43: mol_type = other DNA
ctgtgtaccccttccaggtctgatgagcagttgaaatctggaa

SEQ ID NO: 264 - DNA, 43 bp, synthetic construct
  source 1..43: mol_type = other DNA
ctgtgtaccccttccagggatgagcagttgaaatctggaactg

SEQ ID NO: 265 - DNA, 43 bp, synthetic construct
  source 1..43: mol_type = other DNA
ctgtgtaccccttccagggagcagttgaaatctggaactgcct

SEQ ID NO: 266 - DNA, 43 bp, synthetic construct
  source 1..43: mol_type = other DNA
ctgtgtaccccttccaggcagttgaaatctggaactgcctctg

SEQ ID NO: 267 - DNA, 43 bp, synthetic construct
  source 1..43: mol_type = other DNA
ctgtgtaccccttccaggttgaaatctggaactgcctctgttg

SEQ ID NO: 268 - DNA, 43 bp, synthetic construct
  source 1..43: mol_type = other DNA
ctgtgtaccccttccaggaaatctggaactgcctctgttgtgt

SEQ ID NO: 269 - DNA, 43 bp, synthetic construct
  source 1..43: mol_type = other DNA
ctgtgtaccccttccaggtctggaactgcctctgttgtgtgcc

SEQ ID NO: 270 - DNA, 43 bp, synthetic construct
  source 1..43: mol_type = other DNA
ctgtgtaccccttccaggggaactgcctctgttgtgtgcctgc

SEQ ID NO: 271 - DNA, 43 bp, synthetic construct
  source 1..43: mol_type = other DNA
ctgtgtaccccttccaggactgcctctgttgtgtgcctgctga

SEQ ID NO: 272 - DNA, 43 bp, synthetic construct
  source 1..43: mol_type = other DNA
ctgtgtaccccttccagggcctctgttgtgtgcctgctgaata

SEQ ID NO: 273 - DNA, 43 bp, synthetic construct
  source 1..43: mol_type = other DNA
ctgtgtaccccttccaggtctgttgtgtgcctgctgaataact

SEQ ID NO: 274 - DNA, 43 bp, synthetic construct
  source 1..43: mol_type = other DNA
ctgtgtaccccttccagggttgtgtgcctgctgaataacttct

SEQ ID NO: 275 - DNA, 43 bp, synthetic construct
  source 1..43: mol_type = other DNA
ctgtgtaccccttccagggtgtgcctgctgaataacttctatc

SEQ ID NO: 276 - DNA, 43 bp, synthetic construct
  source 1..43: mol_type = other DNA
cctggaaggggtacacagagttcctgaggaaagaagcaaacag

SEQ ID NO: 277 - DNA, 43 bp, synthetic construct
  source 1..43: mol_type = other DNA
cctggaaggggtacacagcacagttcctgaggaaagaagcaaa

SEQ ID NO: 278 - DNA, 43 bp, synthetic construct
  source 1..43: mol_type = other DNA
cctggaaggggtacacagagccacagttcctgaggaaagaagc

SEQ ID NO: 279 - DNA, 43 bp, synthetic construct
  source 1..43: mol_type = other DNA
cctggaaggggtacacagtgcagccacagttcctgaggaaaga

SEQ ID NO: 280 - DNA, 43 bp, synthetic construct
  source 1..43: mol_type = other DNA
cctggaaggggtacacagtggtgcagccacagttcctgaggaa

SEQ ID NO: 281 - DNA, 43 bp, synthetic construct
  source 1..43: mol_type = other DNA
cctggaaggggtacacagagatggtgcagccacagttcctgag

SEQ ID NO: 282 - DNA, 43 bp, synthetic construct
  source 1..43: mol_type = other DNA
cctggaaggggtacacaggacagatggtgcagccacagttcct

SEQ ID NO: 283 - DNA, 43 bp, synthetic construct
  source 1..43: mol_type = other DNA
cctggaaggggtacacaggaagacagatggtgcagccacagtt

SEQ ID NO: 284 - DNA, 43 bp, synthetic construct
  source 1..43: mol_type = other DNA
cctggaaggggtacacaggatgaagacagatggtgcagccaca

SEQ ID NO: 285 - DNA, 43 bp, synthetic construct
  source 1..43: mol_type = other DNA
cctggaaggggtacacaggaagatgaagacagatggtgcagcc

SEQ ID NO: 286 - DNA, 43 bp, synthetic construct
  source 1..43: mol_type = other DNA
cctggaaggggtacacagcgggaagatgaagacagatggtgca

SEQ ID NO: 287 - DNA, 43 bp, synthetic construct
  source 1..43: mol_type = other DNA
cctggaaggggtacacagtggcgggaagatgaagacagatggt

SEQ ID NO: 288 - DNA, 43 bp, synthetic construct
  source 1..43: mol_type = other DNA
cctggaaggggtacacagagatggcgggaagatgaagacagat

SEQ ID NO: 289 - DNA, 43 bp, synthetic construct
  source 1..43: mol_type = other DNA
cctggaaggggtacacagatcagatggcgggaagatgaagaca

SEQ ID NO: 290 - DNA, 43 bp, synthetic construct
  source 1..43: mol_type = other DNA
cctggaaggggtacacagctcatcagatggcgggaagatgaag

SEQ ID NO: 291 - DNA, 43 bp, synthetic construct
  source 1..43: mol_type = other DNA
cctggaaggggtacacagctgctcatcagatggcgggaagatg

SEQ ID NO: 292 - DNA, 43 bp, synthetic construct
  source 1..43: mol_type = other DNA
cctggaaggggtacacagcaactgctcatcagatggcgggaag

SEQ ID NO: 293 - DNA, 43 bp, synthetic construct
  source 1..43: mol_type = other DNA
cctggaaggggtacacagtttcaactgctcatcagatggcggg

SEQ ID NO: 294 - DNA, 43 bp, synthetic construct
  source 1..43: mol_type = other DNA
cctggaaggggtacacagagatttcaactgctcatcagatggc

SEQ ID NO: 295 - DNA, 43 bp, synthetic construct
  source 1..43: mol_type = other DNA
cctggaaggggtacacagtccagatttcaactgctcatcagat

SEQ ID NO: 296 - DNA, 43 bp, synthetic construct
  source 1..43: mol_type = other DNA
cctggaaggggtacacagagttccagatttcaactgctcatca

SEQ ID NO: 297 - DNA, 43 bp, synthetic construct
  source 1..43: mol_type = other DNA
cctggaaggggtacacagggcagttccagatttcaactgctca

SEQ ID NO: 298 - DNA, 43 bp, synthetic construct
  source 1..43: mol_type = other DNA
cctggaaggggtacacagagaggcagttccagatttcaactgc

SEQ ID NO: 299 - DNA, 43 bp, synthetic construct
  source 1..43: mol_type = other DNA
cctggaaggggtacacagaacagaggcagttccagatttcaac

SEQ ID NO: 300 - DNA, 43 bp, synthetic construct
  source 1..43: mol_type = other DNA
ctgtgtaccccttccaggtgcctgctgaataacttctatccca

SEQ ID NO: 301 - DNA, 43 bp, synthetic construct
  source 1..43: mol_type = other DNA
ctgtgtaccccttccaggctgctgaataacttctatcccagag

SEQ ID NO: 302 - DNA, 43 bp, synthetic construct
  source 1..43: mol_type = other DNA
ctgtgtaccccttccaggctgaataacttctatcccagagagg

SEQ ID NO: 303 - DNA, 43 bp, synthetic construct
  source 1..43: mol_type = other DNA
ctgtgtaccccttccaggaataacttctatcccagagaggcca

SEQ ID NO: 304 - DNA, 43 bp, synthetic construct
  source 1..43: mol_type = other DNA
ctgtgtaccccttccaggttctatcccagagaggccaaagtac

SEQ ID NO: 305 - DNA, 43 bp, synthetic construct; identical to SEQ ID NO: 304
  source 1..43: mol_type = other DNA

SEQ ID NO: 306 - DNA, 43 bp, synthetic construct
  source 1..43: mol_type = other DNA
ctgtgtaccccttccaggtatcccagagaggccaaagtacagt

SEQ ID NO: 307 - DNA, 43 bp, synthetic construct
  source 1..43: mol_type = other DNA
ctgtgtaccccttccaggcccagagaggccaaagtacagtgga

SEQ ID NO: 308 - DNA, 43 bp, synthetic construct
  source 1..43: mol_type = other DNA
ctgtgtaccccttccaggagagaggccaaagtacagtggaagg

SEQ ID NO: 309 - DNA, 43 bp, synthetic construct
  source 1..43: mol_type = other DNA
ctgtgtaccccttccagggaggccaaagtacagtggaaggtgg

SEQ ID NO: 310 - DNA, 43 bp, synthetic construct
  source 1..43: mol_type = other DNA
ctgtgtaccccttccagggccaaagtacagtggaaggtggata

SEQ ID NO: 311 - DNA, 43 bp, synthetic construct
  source 1..43: mol_type = other DNA
ctgtgtaccccttccaggaaagtacagtggaaggtggataacg

SEQ ID NO: 312 - DNA, 43 bp, synthetic construct
  source 1..43: mol_type = other DNA
ctgtgtaccccttccagggtacagtggaaggtggataacgccc

SEQ ID NO: 313 - DNA, 43 bp, synthetic construct
  source 1..43: mol_type = other DNA
ctgtgtaccccttccaggcagtggaaggtggataacgccctcc

SEQ ID NO: 314 - DNA, 43 bp, synthetic construct
  source 1..43: mol_type = other DNA
ctgtgtaccccttccaggtggaaggtggataacgccctccaat

SEQ ID NO: 315 - DNA, 43 bp, synthetic construct
  source 1..43: mol_type = other DNA
ctgtgtaccccttccaggaaggtggataacgccctccaatcgg

SEQ ID NO: 316 - DNA, 43 bp, synthetic construct
  source 1..43: mol_type = other DNA
ctgtgtaccccttccagggtggataacgccctccaatcgggta

SEQ ID NO: 317 - DNA, 43 bp, synthetic construct
  source 1..43: mol_type = other DNA
ctgtgtaccccttccagggataacgccctccaatcgggtaact

SEQ ID NO: 318 - DNA, 43 bp, synthetic construct
  source 1..43: mol_type = other DNA
ctgtgtaccccttccaggaacgccctccaatcgggtaactccc

SEQ ID NO: 319 - DNA, 43 bp, synthetic construct
  source 1..43: mol_type = other DNA
ctgtgtaccccttccagggccctccaatcgggtaactcccagg

SEQ ID NO: 320 - DNA, 43 bp, synthetic construct
  source 1..43: mol_type = other DNA
ctgtgtaccccttccaggctccaatcgggtaactcccaggaga

SEQ ID NO: 321 - DNA, 43 bp, synthetic construct
  source 1..43: mol_type = other DNA
ctgtgtaccccttccaggcaatcgggtaactcccaggagagtg

SEQ ID NO: 322 - DNA, 43 bp, synthetic construct
  source 1..43: mol_type = other DNA
ctgtgtaccccttccaggtcgggtaactcccaggagagtgtca

SEQ ID NO: 323 - DNA, 43 bp, synthetic construct
  source 1..43: mol_type = other DNA
ctgtgtaccccttccaggggtaactcccaggagagtgtcacag

SEQ ID NO: 324 - DNA, 43 bp, synthetic construct
  source 1..43: mol_type = other DNA
ctgtgtaccccttccaggaactcccaggagagtgtcacagagc

SEQ ID NO: 325 - DNA, 43 bp, synthetic construct
  source 1..43: mol_type = other DNA
cctggaaggggtacacagcacaacagaggcagttccagatttc

SEQ ID NO: 326 - DNA, 43 bp, synthetic construct
  source 1..43: mol_type = other DNA
cctggaaggggtacacaggcacacaacagaggcagttccagat

SEQ ID NO: 327 - DNA, 43 bp, synthetic construct
  source 1..43: mol_type = other DNA
cctggaaggggtacacagcaggcacacaacagaggcagttcca

SEQ ID NO: 328 - DNA, 43 bp, synthetic construct
  source 1..43: mol_type = other DNA
cctggaaggggtacacagcagcaggcacacaacagaggcagtt

SEQ ID NO: 329 - DNA, 43 bp, synthetic construct
  source 1..43: mol_type = other DNA
cctggaaggggtacacagattcagcaggcacacaacagaggca

SEQ ID NO: 330 - DNA, 43 bp, synthetic construct
  source 1..43: mol_type = other DNA
cctggaaggggtacacaggttattcagcaggcacacaacagag

SEQ ID NO: 331 - DNA, 43 bp, synthetic construct
  source 1..43: mol_type = other DNA
cctggaaggggtacacaggaagttattcagcaggcacacaaca

SEQ ID NO: 332 - DNA, 43 bp, synthetic construct
  source 1..43: mol_type = other DNA
cctggaaggggtacacagatagaagttattcagcaggcacaca

SEQ ID NO: 333 - DNA, 43 bp, synthetic construct
  source 1..43: mol_type = other DNA
cctggaaggggtacacaggggatagaagttattcagcaggcac

SEQ ID NO: 334 - DNA, 43 bp, synthetic construct
  source 1..43: mol_type = other DNA
cctggaaggggtacacagtctgggatagaagttattcagcagg

SEQ ID NO: 335 - DNA, 43 bp, synthetic construct
  source 1..43: mol_type = other DNA
cctggaaggggtacacagctctctgggatagaagttattcagc

SEQ ID NO: 336 - DNA, 43 bp, synthetic construct
  source 1..43: mol_type = other DNA
cctggaaggggtacacagggcctctctgggatagaagttattc

SEQ ID NO: 337 - DNA, 43 bp, synthetic construct
  source 1..43: mol_type = other DNA
cctggaaggggtacacagtttggcctctctgggatagaagtta

SEQ ID NO: 338 - DNA, 43 bp, synthetic construct
  source 1..43: mol_type = other DNA
cctggaaggggtacacagtactttggcctctctgggatagaag

SEQ ID NO: 339 - DNA, 43 bp, synthetic construct
  source 1..43: mol_type = other DNA
cctggaaggggtacacagctgtactttggcctctctgggatag

SEQ ID NO: 340 - DNA, 43 bp, synthetic construct
  source 1..43: mol_type = other DNA
cctggaaggggtacacagccactgtactttggcctctctggga

SEQ ID NO: 341 - DNA, 43 bp, synthetic construct
  source 1..43: mol_type = other DNA
cctggaaggggtacacagcttccactgtactttggcctctctg

SEQ ID NO: 342 - DNA, 43 bp, synthetic construct
  source 1..43: mol_type = other DNA
cctggaaggggtacacagcaccttccactgtactttggcctct

SEQ ID NO: 343 - DNA, 43 bp, synthetic construct
  source 1..43: mol_type = other DNA
cctggaaggggtacacagatccaccttccactgtactttggcc

SEQ ID NO: 344 - DNA, 43 bp, synthetic construct
  source 1..43: mol_type = other DNA
cctggaaggggtacacaggttatccaccttccactgtactttg

SEQ ID NO: 345 - DNA, 43 bp, synthetic construct
  source 1..43: mol_type = other DNA
cctggaaggggtacacagggcgttatccaccttccactgtact

SEQ ID NO: 346 - DNA, 43 bp, synthetic construct
  source 1..43: mol_type = other DNA
cctggaaggggtacacaggagggcgttatccaccttccactgt

SEQ ID NO: 347 - DNA, 43 bp, synthetic construct
  source 1..43: mol_type = other DNA
cctggaaggggtacacagttggagggcgttatccaccttccac

SEQ ID NO: 348 - DNA, 43 bp, synthetic construct
  source 1..43: mol_type = other DNA
cctggaaggggtacacagcgattggagggcgttatccaccttc

SEQ ID NO: 349 - DNA, 43 bp, synthetic construct
  source 1..43: mol_type = other DNA
cctggaaggggtacacagacccgattggagggcgttatccacc

SEQ ID NO: 350 - DNA, 43 bp, synthetic construct
  source 1..43: mol_type = other DNA
ctgtgtaccccttccaggtcccaggagagtgtcacagagcagg

SEQ ID NO: 351 - DNA, 43 bp, synthetic construct
  source 1..43: mol_type = other DNA
ctgtgtaccccttccaggcaggagagtgtcacagagcaggaca

SEQ ID NO: 352 - DNA, 43 bp, synthetic construct
  source 1..43: mol_type = other DNA
ctgtgtaccccttccagggagagtgtcacagagcaggacagca

SEQ ID NO: 353 - DNA, 43 bp, synthetic construct
  source 1..43: mol_type = other DNA
ctgtgtaccccttccaggagtgtcacagagcaggacagcaagg

SEQ ID NO: 354 - DNA, 43 bp, synthetic construct
  source 1..43: mol_type = other DNA
ctgtgtaccccttccagggtcacagagcaggacagcaaggaca

SEQ ID NO: 355 - DNA, 43 bp, synthetic construct
  source 1..43: mol_type = other DNA
ctgtgtaccccttccaggacagagcaggacagcaaggacagca

SEQ ID NO: 356 - DNA, 43 bp, synthetic construct
  source 1..43: mol_type = other DNA
ctgtgtaccccttccagggagcaggacagcaaggacagcacct

SEQ ID NO: 357 - DNA, 43 bp, synthetic construct
  source 1..43: mol_type = other DNA
ctgtgtaccccttccaggcaggacagcaaggacagcacctaca

SEQ ID NO: 358 - DNA, 43 bp, synthetic construct
  source 1..43: mol_type = other DNA
ctgtgtaccccttccagggacagcaaggacagcacctacagcc

SEQ ID NO: 359 - DNA, 43 bp, synthetic construct
  source 1..43: mol_type = other DNA
ctgtgtaccccttccaggagcaaggacagcacctacagcctca

SEQ ID NO: 360 - DNA, 43 bp, synthetic construct
  source 1..43: mol_type = other DNA
ctgtgtaccccttccaggaaggacagcacctacagcctcagca

SEQ ID NO: 361 - DNA, 43 bp, synthetic construct
  source 1..43: mol_type = other DNA
ctgtgtaccccttccagggacagcacctacagcctcagcagca

SEQ ID NO: 362 - DNA, 43 bp, synthetic construct
  source 1..43: mol_type = other DNA
ctgtgtaccccttccaggagcacctacagcctcagcagcaccc

SEQ ID NO: 363 - DNA, 43 bp, synthetic construct
  source 1..43: mol_type = other DNA
ctgtgtaccccttccaggacctacagcctcagcagcaccctga

SEQ ID NO: 364 - DNA, 43 bp, synthetic construct
  source 1..43: mol_type = other DNA
ctgtgtaccccttccaggtacagcctcagcagcaccctgacgc

SEQ ID NO: 365 - DNA, 43 bp, synthetic construct
  source 1..43: mol_type = other DNA
ctgtgtaccccttccaggagcctcagcagcaccctgacgctga

SEQ ID NO: 366 - DNA, 43 bp, synthetic construct
  source 1..43: mol_type = other DNA
ctgtgtaccccttccaggctcagcagcaccctgacgctgagca

SEQ ID NO: 367 - DNA, 43 bp, synthetic construct
  source 1..43: mol_type = other DNA
ctgtgtaccccttccaggagcagcaccctgacgctgagcaaag

SEQ ID NO: 368 - DNA, 43 bp, synthetic construct
  source 1..43: mol_type = other DNA
ctgtgtaccccttccaggagcaccctgacgctgagcaaagcag

SEQ ID NO: 369 - DNA, 43 bp, synthetic construct
  source 1..43: mol_type = other DNA
ctgtgtaccccttccaggaccctgacgctgagcaaagcagact

SEQ ID NO: 370 - DNA, 43 bp, synthetic construct
  source 1..43: mol_type = other DNA
ctgtgtaccccttccaggctgacgctgagcaaagcagactacg

SEQ ID NO: 371 - DNA, 43 bp, synthetic construct
  source 1..43: mol_type = other DNA
ctgtgtaccccttccaggacgctgagcaaagcagactacgaga

SEQ ID NO: 372 - DNA, 43 bp, synthetic construct
  source 1..43: mol_type = other DNA
ctgtgtaccccttccaggctgagcaaagcagactacgagaaac

SEQ ID NO: 373 - DNA, 43 bp, synthetic construct
  source 1..43: mol_type = other DNA
ctgtgtaccccttccaggagcaaagcagactacgagaaacaca

SEQ ID NO: 374 - DNA, 43 bp, synthetic construct
  source 1..43: mol_type = other DNA
ctgtgtaccccttccaggaaagcagactacgagaaacacaaag

SEQ ID NO: 375 - DNA, 43 bp, synthetic construct
  source 1..43: mol_type = other DNA
cctggaaggggtacacaggttacccgattggagggcgttatcc

SEQ ID NO: 376 - DNA, 43 bp, synthetic construct
  source 1..43: mol_type = other DNA
cctggaaggggtacacagggagttacccgattggagggcgtta

SEQ ID NO: 377 - DNA, 43 bp, synthetic construct
  source 1..43: mol_type = other DNA
cctggaaggggtacacagctgggagttacccgattggagggcg

SEQ ID NO: 378 - DNA, 43 bp, synthetic construct
  source 1..43: mol_type = other DNA
cctggaaggggtacacagctcctgggagttacccgattggagg

SEQ ID NO: 379 - DNA, 43 bp, synthetic construct
  source 1..43: mol_type = other DNA
cctggaaggggtacacagactctcctgggagttacccgattgg

SEQ ID NO: 380 - DNA, 43 bp, synthetic construct
  source 1..43: mol_type = other DNA
cctggaaggggtacacaggacactctcctgggagttacccgat

SEQ ID NO: 381 - DNA, 43 bp, synthetic construct
  source 1..43: mol_type = other DNA
cctggaaggggtacacagtgtgacactctcctgggagttaccc

SEQ ID NO: 382 - DNA, 43 bp, synthetic construct
  source 1..43: mol_type = other DNA
cctggaaggggtacacagctctgtgacactctcctgggagtta

SEQ ID NO: 383 - DNA, 43 bp, synthetic construct
  source 1..43: mol_type = other DNA
cctggaaggggtacacagctgctctgtgacactctcctgggag

SEQ ID NO: 384 - DNA, 43 bp, synthetic construct
  source 1..43: mol_type = other DNA
cctggaaggggtacacaggtcctgctctgtgacactctcctgg

SEQ ID NO: 385 - DNA, 43 bp, synthetic construct
  source 1..43: mol_type = other DNA
cctggaaggggtacacaggctgtcctgctctgtgacactctcc

SEQ ID NO: 386 - DNA, 43 bp, synthetic construct
  source 1..43: mol_type = other DNA
cctggaaggggtacacagcttgctgtcctgctctgtgacactc

SEQ ID NO: 387 - DNA, 43 bp, synthetic construct
  source 1..43: mol_type = other DNA
cctggaaggggtacacaggtccttgctgtcctgctctgtgaca

SEQ ID NO: 388 - DNA, 43 bp, synthetic construct
  source 1..43: mol_type = other DNA
cctggaaggggtacacaggctgtccttgctgtcctgctctgtg

SEQ ID NO: 389 - DNA, 43 bp, synthetic construct
  source 1..43: mol_type = other DNA
cctggaaggggtacacagggtgctgtccttgctgtcctgctct

SEQ ID NO: 390 - DNA, 43 bp, synthetic construct
  source 1..43: mol_type = other DNA
cctggaaggggtacacaggtaggtgctgtccttgctgtcctgc

SEQ ID NO: 391 - DNA, 43 bp, synthetic construct
  source 1..43: mol_type = other DNA
cctggaaggggtacacaggctgtaggtgctgtccttgctgtcc

SEQ ID NO: 392 - DNA, 43 bp, synthetic construct
  source 1..43: mol_type = other DNA
cctggaaggggtacacaggaggctgtaggtgctgtccttgctg

SEQ ID NO: 393 - DNA, 43 bp, synthetic construct
  source 1..43: mol_type = other DNA
cctggaaggggtacacaggctgaggctgtaggtgctgtccttg

SEQ ID NO: 394 - DNA, 43 bp, synthetic construct
  source 1..43: mol_type = other DNA
cctggaaggggtacacaggctgctgaggctgtaggtgctgtcc

SEQ ID NO: 395 - DNA, 43 bp, synthetic construct
  source 1..43: mol_type = other DNA
cctggaaggggtacacagggtgctgctgaggctgtaggtgctg

SEQ ID NO: 396 - DNA, 43 bp, synthetic construct
  source 1..43: mol_type = other DNA
cctggaaggggtacacagcagggtgctgctgaggctgtaggtg

SEQ ID NO: 397 - DNA, 43 bp, synthetic construct
  source 1..43: mol_type = other DNA
cctggaaggggtacacagcgtcagggtgctgctgaggctgtag

SEQ ID NO: 398 - DNA, 43 bp, synthetic construct
  source 1..43: mol_type = other DNA
cctggaaggggtacacagcagcgtcagggtgctgctgaggctg

SEQ ID NO: 399 - DNA, 43 bp, synthetic construct
  source 1..43: mol_type = other DNA
cctggaaggggtacacaggctcagcgtcagggtgctgctgagg

SEQ ID NO: 400 - DNA, 43 bp, synthetic construct
  source 1..43: mol_type = other DNA
ctgtgtaccccttccagggcagactacgagaaacacaaagtct

SEQ ID NO: 401 - DNA, 43 bp, synthetic construct
  source 1..43: mol_type = other DNA
ctgtgtaccccttccagggactacgagaaacacaaagtctacg

SEQ ID NO: 402 - DNA, 43 bp, synthetic construct
  source 1..43: mol_type = other DNA
ctgtgtaccccttccaggtacgagaaacacaaagtctacgcct

SEQ ID NO: 403 - DNA, 43 bp, synthetic construct
  source 1..43: mol_type = other DNA
ctgtgtaccccttccagggagaaacacaaagtctacgcctgcg

SEQ ID NO: 404 - DNA, 43 bp, synthetic construct
  source 1..43: mol_type = other DNA
ctgtgtaccccttccaggaaacacaaagtctacgcctgcgaag

SEQ ID NO: 405 - DNA, 43 bp, synthetic construct
  source 1..43: mol_type = other DNA
ctgtgtaccccttccaggcacaaagtctacgcctgcgaagtca

SEQ ID NO: 406 - DNA, 43 bp, synthetic construct
  source 1..43: mol_type = other DNA
ctgtgtaccccttccaggaaagtctacgcctgcgaagtcaccc

SEQ ID NO: 407 - DNA, 43 bp, synthetic construct
  source 1..43: mol_type = other DNA
ctgtgtaccccttccagggtctacgcctgcgaagtcacccatc

SEQ ID NO: 408 - DNA, 43 bp, synthetic construct
  source 1..43: mol_type = other DNA
ctgtgtaccccttccaggtacgcctgcgaagtcacccatcagg

SEQ ID NO: 409 - DNA, 43 bp, synthetic construct
  source 1..43: mol_type = other DNA
ctgtgtaccccttccagggcctgcgaagtcacccatcagggcc

SEQ ID NO: 410 - DNA, 43 bp, synthetic construct
  source 1..43: mol_type = other DNA
ctgtgtaccccttccaggtgcgaagtcacccatcagggcctga

SEQ ID NO: 411 - DNA, 43 bp, synthetic construct
  source 1..43: mol_type = other DNA
ctgtgtaccccttccagggaagtcacccatcagggcctgagct

SEQ ID NO: 412 - DNA, 43 bp, synthetic construct
  source 1..43: mol_type = other DNA
ctgtgtaccccttccagggtcacccatcagggcctgagctcgc

SEQ ID NO: 413 - DNA, 43 bp, synthetic construct
  source 1..43: mol_type = other DNA
ctgtgtaccccttccaggacccatcagggcctgagctcgcccg

SEQ ID NO: 414 - DNA, 43 bp, synthetic construct
  source 1..43: mol_type = other DNA
ctgtgtaccccttccaggcatcagggcctgagctcgcccgtca

SEQ ID NO: 415 - DNA, 43 bp, synthetic construct
  source 1..43: mol_type = other DNA
ctgtgtaccccttccaggcagggcctgagctcgcccgtcacaa

SEQ ID NO: 416 - DNA, 43 bp, synthetic construct
  source 1..43: mol_type = other DNA
ctgtgtaccccttccaggggcctgagctcgcccgtcacaaaga

SEQ ID NO: 417 - DNA, 43 bp, synthetic construct
  source 1..43: mol_type = other DNA
ctgtgtaccccttccaggctgagctcgcccgtcacaaagagct

SEQ ID NO: 418 - DNA, 43 bp, synthetic construct
  source 1..43: mol_type = other DNA
ctgtgtaccccttccaggagctcgcccgtcacaaagagcttca

SEQ ID NO: 419 - DNA, 43 bp, synthetic construct
  source 1..43: mol_type = other DNA
ctgtgtaccccttccaggtcgcccgtcacaaagagcttcaaca

SEQ ID NO: 420 - DNA, 43 bp, synthetic construct
  source 1..43: mol_type = other DNA
ctgtgtaccccttccaggcccgtcacaaagagcttcaacaggg

SEQ ID NO: 421 - DNA, 43 bp, synthetic construct
  source 1..43: mol_type = other DNA
ctgtgtaccccttccagggtcacaaagagcttcaacaggggag

SEQ ID NO: 422 - DNA, 43 bp, synthetic construct
  source 1..43: mol_type = other DNA
ctgtgtaccccttccaggacaaagagcttcaacaggggagagt

SEQ ID NO: 423 - DNA, 43 bp, synthetic construct
  source 1..43: mol_type = other DNA
ctgtgtaccccttccaggaagagcttcaacaggggagagtgtt

SEQ ID NO: 424 - DNA, 43 bp, synthetic construct
  source 1..43: mol_type = other DNA
ctgtgtaccccttccaggagcttcaacaggggagagtgttagc

SEQ ID NO: 425 - DNA, 43 bp, synthetic construct
  source 1..43: mol_type = other DNA
cctggaaggggtacacagtttgctcagcgtcagggtgctgctg

SEQ ID NO: 426 - DNA, 43 bp, synthetic construct
  source 1..43: mol_type = other DNA
cctggaaggggtacacagtgctttgctcagcgtcagggtgctg

SEQ ID NO: 427 - DNA, 43 bp, synthetic construct
  source 1..43: mol_type = other DNA
cctggaaggggtacacaggtctgctttgctcagcgtcagggtg

SEQ ID NO: 428 - DNA, 43 bp, synthetic construct
  source 1..43: mol_type = other DNA
cctggaaggggtacacaggtagtctgctttgctcagcgtcagg

SEQ ID NO: 429 - DNA, 43 bp, synthetic construct
  source 1..43: mol_type = other DNA
cctggaaggggtacacagctcgtagtctgctttgctcagcgtc

SEQ ID NO: 430 - DNA, 43 bp, synthetic construct
  source 1..43: mol_type = other DNA
cctggaaggggtacacagtttctcgtagtctgctttgctcagc

SEQ ID NO: 431 - DNA, 43 bp, synthetic construct
  source 1..43: mol_type = other DNA
cctggaaggggtacacaggtgtttctcgtagtctgctttgctc

SEQ ID NO: 432 - DNA, 43 bp, synthetic construct
  source 1..43: mol_type = other DNA
cctggaaggggtacacagtttgtgtttctcgtagtctgctttg

SEQ ID NO: 433 - DNA, 43 bp, synthetic construct
  source 1..43: mol_type = other DNA
cctggaaggggtacacaggactttgtgtttctcgtagtctgct

SEQ ID NO: 434 - DNA, 43 bp, synthetic construct
  source 1..43: mol_type = other DNA
cctggaaggggtacacaggtagactttgtgtttctcgtagtct

SEQ ID NO: 435 - DNA, 43 bp, synthetic construct
  source 1..43: mol_type = other DNA
cctggaaggggtacacagggcgtagactttgtgtttctcgtag

SEQ ID NO: 436 - DNA, 43 bp, synthetic construct
  source 1..43: mol_type = other DNA
cctggaaggggtacacaggcaggcgtagactttgtgtttctcg

SEQ ID NO: 437 - DNA, 43 bp, synthetic construct
  source 1..43: mol_type = other DNA
cctggaaggggtacacagttcgcaggcgtagactttgtgtttc

SEQ ID NO: 438 - DNA, 43 bp, synthetic construct
  source 1..43: mol_type = other DNA
cctggaaggggtacacaggacttcgcaggcgtagactttgtgt

SEQ ID NO: 439 - DNA, 43 bp, synthetic construct
  source 1..43: mol_type = other DNA
cctggaaggggtacacagggtgacttcgcaggcgtagactttg

SEQ ID NO: 440 - DNA, 43 bp, synthetic construct
  source 1..43: mol_type = other DNA
cctggaaggggtacacagatgggtgacttcgcaggcgtagact

SEQ ID NO: 441 - DNA, 43 bp, synthetic construct
  source 1..43: mol_type = other DNA
cctggaaggggtacacagctgatgggtgacttcgcaggcgtag

SEQ ID NO: 442 - DNA, 43 bp, synthetic construct
  source 1..43: mol_type = other DNA
cctggaaggggtacacaggccctgatgggtgacttcgcaggcg

SEQ ID NO: 443 - DNA, 43 bp, synthetic construct
  source 1..43: mol_type = other DNA
cctggaaggggtacacagcaggccctgatgggtgacttcgcag

SEQ ID NO: 444 - DNA, 43 bp, synthetic construct
  source 1..43: mol_type = other DNA
cctggaaggggtacacaggctcaggccctgatgggtgacttcg

SEQ ID NO: 445 - DNA, 43 bp, synthetic construct
  source 1..43: mol_type = other DNA
cctggaaggggtacacagcgagctcaggccctgatgggtgact

SEQ ID NO: 446 - DNA, 43 bp, synthetic construct
  source 1..43: mol_type = other DNA
cctggaaggggtacacaggggcgagctcaggccctgatgggtg

SEQ ID NO: 447 - DNA, 43 bp, synthetic construct
  source 1..43: mol_type = other DNA
cctggaaggggtacacaggacgggcgagctcaggccctgatgg

SEQ ID NO: 448 - DNA, 43 bp, synthetic construct
  source 1..43: mol_type = other DNA
cctggaaggggtacacagtgtgacgggcgagctcaggccctga

SEQ ID NO: 449 - DNA, 43 bp, synthetic construct
  source 1..43: mol_type = other DNA
cctggaaggggtacacagctttgtgacgggcgagctcaggccc

SEQ ID NO: 450 - DNA, 43 bp, synthetic construct
  source 1..43: mol_type = other DNA
ctgtgtaccccttccaggttcaacaggggagagtgttagcctg

SEQ ID NO: 451 - DNA, 43 bp, synthetic construct
  source 1..43: mol_type = other DNA
ctgtgtaccccttccaggaacaggggagagtgttagcctgcag

SEQ ID NO: 452 - DNA, 43 bp, synthetic construct
  source 1..43: mol_type = other DNA
ctgtgtaccccttccaggaggggagagtgttagcctgcaggca

SEQ ID NO: 453 - DNA, 43 bp, synthetic construct
  source 1..43: mol_type = other DNA
ctgtgtaccccttccaggggagagtgttagcctgcaggcatga

SEQ ID NO: 454 - DNA, 43 bp, synthetic construct
  source 1..43: mol_type = other DNA
ctgtgtaccccttccagggagtgttagcctgcaggcatgatca

SEQ ID NO: 455 - DNA, 43 bp, synthetic construct
  source 1..43: mol_type = other DNA
ctgtgtaccccttccaggtgttagcctgcaggcatgatcataa

SEQ ID NO: 456 - DNA, 43 bp, synthetic construct
  source 1..43: mol_type = other DNA
ctgtgtaccccttccaggtagcctgcaggcatgatcataatca

SEQ ID NO: 457 - DNA, 43 bp, synthetic construct
  source 1..43: mol_type = other DNA
cctggaaggggtacacaggctctttgtgacgggcgagctcagg

SEQ ID NO: 458 - DNA, 43 bp, synthetic construct
  source 1..43: mol_type = other DNA
cctggaaggggtacacaggaagctctttgtgacgggcgagctc

SEQ ID NO: 459 - DNA, 43 bp, synthetic construct
  source 1..43: mol_type = other DNA
cctggaaggggtacacaggttgaagctctttgtgacgggcgag

SEQ ID NO: 460 - DNA, 43 bp, synthetic construct
  source 1..43: mol_type = other DNA
cctggaaggggtacacagcctgttgaagctctttgtgacgggc

SEQ ID NO: 461 - DNA, 43 bp, synthetic construct
  source 1..43: mol_type = other DNA
cctggaaggggtacacagtcccctgttgaagctctttgtgacg

SEQ ID NO: 462 - DNA, 43 bp, synthetic construct
  source 1..43: mol_type = other DNA
cctggaaggggtacacagctctcccctgttgaagctctttgtg

SEQ ID NO: 463 - DNA, 43 bp, synthetic construct
  source 1..43: mol_type = other DNA
cctggaaggggtacacagacactctcccctgttgaagctcttt

SEQ ID NO: 464 - protein, 5 aa, synthetic construct
TVAAP

SEQ ID NO: 465 - protein, 5 aa, synthetic construct
YPREA

SEQ ID NO: 466 - protein, 4 aa, synthetic construct
PREA

SEQ ID NO: 467 - protein, 8 aa, synthetic construct
DNALQSGN

SEQ ID NO: 468 - protein, 9 aa, synthetic construct
TEQDSKDST

SEQ ID NO: 469 - protein, 6 aa, synthetic construct
HQGLSS

SEQ ID NO: 470 - protein, 4 aa, synthetic construct
RGEC

SEQ ID NO: 471 - protein, 6 aa, synthetic construct
TVAAPS

SEQ ID NO: 472 - protein, 4 aa, synthetic construct
SGTA

SEQ ID NO: 473 - protein, 8 aa, synthetic construct
NNFYPREA

SEQ ID NO: 474 - protein, 11 aa, synthetic construct
VDNALQSGNSQ

SEQ ID NO: 475 - protein, 11 aa, synthetic construct
VTEQDSKDSTY

SEQ ID NO: 476 - protein, 8 aa, synthetic construct
THQGLSSP

SEQ ID NO: 477 - protein, 5 aa, synthetic construct
NRGEC

SEQ ID NO: 478 - skipped

SEQ ID NO: 479 - protein, 9 aa, synthetic construct
  VARIANT 2: note = Xaa at position 2 may be present or absent, and when present may be any amino acid
  MOD_RES 3: note = FORMYLATION
  VARIANT 4: note = Xaa can be any naturally occurring amino acid
  VARIANT 5: note = Xaa at position 5 is Pro or Ala
  VARIANT 6: note = Xaa can be any naturally occurring amino acid
  VARIANT 7: note = Xaa at position 7 is an aliphatic or baisc amino acid
TXGXXXXVA

SEQ ID NO: 480 - protein, 10 aa, synthetic construct
  VARIANT 3: note = Xaa can be any naturally occurring amino acid
  MOD_RES 4: note = FORMYLATION
  VARIANT 5..8: note = Xaa can be any naturally occurring amino acid
TVXGXXXXAA

SEQ ID NO: 481 - protein, 10 aa, synthetic construct
  VARIANT 3: note = Xaa can be any naturally occurring amino acid
  MOD_RES 4: note = FORMYLATION
  VARIANT 5..8: note = Xaa can be any naturally occurring amino acid
VAXGXXXXAP

SEQ ID NO: 482 - protein, 11 aa, synthetic construct
  VARIANT 3: note = Xaa can be any naturally occurring amino acid
  MOD_RES 4: note = FORMYLATION
  VARIANT 5..8: note = Xaa can be any naturally occurring amino acid
AAXGXXXXPSV

SEQ ID NO: 483 - protein, 11 aa, synthetic construct
  VARIANT 3: note = Xaa can be any nautrally occuring amino acid
  MOD_RES 4: note = FORMYLATION
  VARIANT 5..8: note = Xaa can be any naturally occurring amino acid
APXGXXXXSVF

SEQ ID NO: 484 - protein, 10 aa, synthetic construct
  VARIANT 3: note = Xaa can be any nautrally occurring amino acid
  MOD_RES 4: note = FORMYLATION
  VARIANT 5..8: note = Xaa can be any naturally occurring amino acid
PSXGXXXXVF

SEQ ID NO: 485 - protein, 10 aa, synthetic construct
  VARIANT 3: note = Xaa can be any nautrally occuring amino acid
  MOD_RES 4: note = FORMYLATION
  VARIANT 5..8: note = Xaa can be any naturally occurring amino acid
KSXGXXXXGT

SEQ ID NO: 486 - protein, 11 aa, synthetic construct
  MOD_RES 3: note = FORMYLATION
  VARIANT 4: note = Xaa can be any naturally occurring amino acid
  VARIANT 6..9: note = Xaa can be any naturally occurring amino acid
KSGXGXXXXTA

SEQ ID NO: 487 - protein, 10 aa, synthetic construct
  VARIANT 3: note = Xaa can be any naturally occurring amino acid
  MOD_RES 4: note = FORMYLATION
  VARIANT 5..8: note = Xaa can be any naturally occurring amino acid
GTXGXXXXAS

SEQ ID NO: 488 - protein, 11 aa, synthetic construct
  VARIANT 3: note = Xaa can be any naturally occurring amino acid
  MOD_RES 4: note = FORMYLATION
  VARIANT 5..8: note = Xaa can be any naturally occurring amino acid
TAXGXXXXSVV

SEQ ID NO: 489 - protein, 10 aa, synthetic construct
  VARIANT 3: note = Xaa can be any naturally occurring amino acid
  MOD_RES 4: note = FORMYLATION
  VARIANT 5..8: note = Xaa can be any naturally occurring amino acid
LNXGXXXXNF

SEQ ID NO: 490 - protein, 10 aa, synthetic construct
  VARIANT 3: note = Xaa can be any naturally occurring amino acid
  MOD_RES 4: note = FORMYLATION
  VARIANT 5..8: note = Xaa can be any naturally occurring amino acid
NNXGXXXXFY

SEQ ID NO: 491 - protein, 10 aa, synthetic construct
  VARIANT 3: note = Xaa can be any naturally occurring amino acid
  MOD_RES 4: note = FORMYLATION
  VARIANT 5..8: note = Xaa can be any naturally occurring amino acid
NFXGXXXXYP

SEQ ID NO: 492 - protein, 10 aa, synthetic construct
  VARIANT 3: note = Xaa can be any naturally occurring amino acid
  MOD_RES 4: note = FORMYLATION
  VARIANT 5..8: note = Xaa can be any naturally occurring amino acid
FYXGXXXXPR

SEQ ID NO: 493 - protein, 11 aa, synthetic construct
  VARIANT 4: note = Xaa can be any naturally occurring amino acid
  MOD_RES 5: note = FORMYLATION
  VARIANT 6..9: note = Xaa can be any naturally occurring amino acid
WKVXGXXXXDN

SEQ ID NO: 494 - protein, 10 aa, synthetic construct
  VARIANT 3: note = Xaa can be any naturally occurring amino acid
  MOD_RES 4: note = FORMYLATION
  VARIANT 5..8: note = Xaa can be any naturally occurring amino acid
  VARIANT 10: note = Xaa can be Ala or Val
VDXGXXXXNX

SEQ ID NO: 495 - protein, 10 aa, synthetic construct
  VARIANT 2: note = Xaa can be Ala or Val
  MOD_RES 3: note = FORMYLATION
  VARIANT 4..8: note = Xaa can be any naturally occurring amino acid
NXGXXXXXLQ

SEQ ID NO: 496 - protein, 10 aa, synthetic construct
  VARIANT 1: note = Xaa can be Ala or Val
  VARIANT 3: note = Xaa can be any naturally occurring amino acid
  MOD_RES 4: note = FORMYLATION
  VARIANT 5..8: note = Xaa can be any naturally occurring amino acid
XLXGXXXXQS

SEQ ID NO: 497 - protein, 11 aa, synthetic construct
  VARIANT 3: note = Xaa can be any naturaly occurring amino acid
  MOD_RES 4: note = FORMYLATION
  VARIANT 5..8: note = Xaa can be any naturally occurring amino acid
LQXGXXXXSGN

SEQ ID NO: 498 - protein, 10 aa, synthetic construct
  VARIANT 3: note = Xaa can be any naturally occurring amino acid
  MOD_RES 4: note = FORMYLATION
  VARIANT 5..8: note = Xaa can be any naturally occurring amino acid
QSXGXXXXGN

SEQ ID NO: 499 - protein, 11 aa, synthetic construct
  VARIANT 4: note = Xaa can be any naturally occurring amino acid
  MOD_RES 5: note = FORMYLATION
  VARIANT 6..9: note = Xaa can be any naturally occurring amino acid
QSGXGXXXXNS

SEQ ID NO: 500 - protein, 10 aa, synthetic construct
  VARIANT 3: note = Xaa can be any naturally occurring amino acid
  MOD_RES 4: note = FORMYLATION
  VARIANT 5..8: note = Xaa can be any naturally occurring amino acid
GNXGXXXXSQ

SEQ ID NO: 501 - protein, 10 aa, synthetic construct
  VARIANT 3: note = Xaa can be any naturally occurring amino acid
  MOD_RES 4: note = FORMYLATION
  VARIANT 5..8: note = Xaa can be any naturally occurring amino acid
NSXGXXXXQE

SEQ ID NO: 502 - protein, 10 aa, synthetic construct
  VARIANT 3: note = Xaa can be any naturally occurring amino acid
  MOD_RES 4: note = FORMYLATION
  VARIANT 5..8: note = Xaa can be any naturally occurring amino acid
SQXGXXXXES

SEQ ID NO: 503 - protein, 11 aa, synthetic construct
  VARIANT 4: note = Xaa can be any naturally occurring amino acid
  MOD_RES 5: note = FORMYLATION
  VARIANT 6..9: note = Xaa can be any naturally occurring amino acid
QDSXGXXXXKD

SEQ ID NO: 504 - protein, 11 aa, synthetic construct
  VARIANT 3: note = Xaa can be any naturally occurring amino acid
  MOD_RES 4: note = FORMYLATION
  VARIANT 5..8: note = Xaa can be any naturally occurring amino acid
KDXGXXXXSTY

SEQ ID NO: 505 - protein, 11 aa, synthetic construct
  VARIANT 4: note = Xaa can be any naturally occurring amino acid
  MOD_RES 5: note = FORMYLATION
  VARIANT 6..9: note = Xaa can be any naturally occurring amino acid
KDSXGXXXXTY

SEQ ID NO: 506 - protein, 11 aa, synthetic construct
  VARIANT 4: note = Xaa can be any naturally occurring amino acid
  MOD_RES 5: note = FORMYLATION
  VARIANT 6..9: note = Xaa can be any naturally occurring amino acid
DSTXGXXXXYS

SEQ ID NO: 507 - protein, 10 aa, synthetic construct
  VARIANT 3: note = Xaa can be any naturally occurring amino acid
  MOD_RES 4: note = FORMYLATION
  VARIANT 5..8: note = Xaa can be any naturally occurring amino acid
TYXGXXXXSL

SEQ ID NO: 508 - protein, 11 aa, synthetic construct
  VARIANT 4: note = Xaa can be any naturally occurring amino acid
  MOD_RES 5: note = FORMYLATION
  VARIANT 6..9: note = Xaa can be any naturally occurring amino acid
EVTXGXXXXHQ

SEQ ID NO: 509 - protein, 10 aa, synthetic construct
  VARIANT 3: note = Xaa can be any naturally occurring amino acid
  MOD_RES 4: note = FORMYLATION
  VARIANT 5..8: note = Xaa can be any naturally occurring amino acid
THXGXXXXQG

SEQ ID NO: 510 - protein, 10 aa, synthetic construct
  VARIANT 3: note = Xaa can be any naturally occurring amino acid
  MOD_RES 4: note = FORMYLATION
  VARIANT 5..8: note = Xaa can be any naturallyo ccurring amino acid
HQXGXXXXGL

SEQ ID NO: 511 - protein, 12 aa, synthetic construct
  VARIANT 3: note = Xaa can be any naturally occurring amino acid
  MOD_RES 4: note = FORMYLATION
  VARIANT 5..8: note = Xaa can be any naturally occurring amino acid
QGXGXXXXLSSP

SEQ ID NO: 512 - protein, 10 aa, synthetic construct
  VARIANT 3: note = Xaa can be any naturally occurring amino acid
  MOD_RES 4: note = FORMYLATION
  VARIANT 5..8: note = Xaa can be any naturally occurring amino acid
GLXGXXXXSS

SEQ ID NO: 513 - protein, 11 aa, synthetic construct
  VARIANT 4: note = Xaa can be any naturally occurring amino acid
  MOD_RES 5: note = FORMYLATION
  VARIANT 6..9: note = Xaa can be any naturally occurring amino acid
GLSXGXXXXSP

SEQ ID NO: 514 - protein, 12 aa, synthetic construct
  VARIANT 5: note = Xaa can be any naturally occurring amino acid
  MOD_RES 6: note = FORMYLATION
  VARIANT 7..10: note = Xaa can be any naturally occurring amino acid
GLSSXGXXXXPV

SEQ ID NO: 515 - protein, 10 aa, synthetic construct
  VARIANT 3: note = Xaa can be any naturally occurring amino acid
  MOD_RES 4: note = FORMYLATION
  VARIANT 5..8: note = Xaa can eb any naturally occurring amino acid
SPXGXXXXVT